SEQUENCE LISTING (91 sequences)

SEQ ID NO: 1 - DNA, 23 bp, Artificial Sequence
  target sequence
tgggggttagacccaatatcagg

SEQ ID NO: 2 - DNA, 23 bp, Artificial Sequence
  target sequence
ccttcctagtctcctgatattgg

SEQ ID NO: 3 - DNA, 23 bp, Artificial Sequence
  target sequence
ccaatatcaggagactaggaagg

SEQ ID NO: 4 - DNA, 23 bp, Artificial Sequence
  target sequence
agataaggaatctgcctaacagg

SEQ ID NO: 5 - DNA, 23 bp, Artificial Sequence
  target sequence
tgttaggcagattccttatctgg

SEQ ID NO: 6 - DNA, 23 bp, Artificial Sequence
  target sequence
tgggggtgtgtcaccagataagg

SEQ ID NO: 7 - DNA, 23 bp, Artificial Sequence
  target sequence
tctccttgccagaacctctaagg

SEQ ID NO: 8 - DNA, 23 bp, Artificial Sequence
  target sequence
cctctaaggtttgcttacgatgg

SEQ ID NO: 9 - DNA, 23 bp, Artificial Sequence
  target sequence
aaaccttagaggttctggcaagg

SEQ ID NO: 10 - DNA, 23 bp, Artificial Sequence
  target sequence
taagcaaaccttagaggttctgg

SEQ ID NO: 11 - DNA, 23 bp, Artificial Sequence
  target sequence
gtgacctgcccggttctcagtgg

SEQ ID NO: 12 - DNA, 23 bp, Artificial Sequence
  target sequence
ggggggatgcgtgacctgcccgg

SEQ ID NO: 13 - DNA, 23 bp, Artificial Sequence
  target sequence
gttctgggagagggtagcgcagg

SEQ ID NO: 14 - DNA, 23 bp, Artificial Sequence
  target sequence
gctgctctgacgcggccgtctgg

SEQ ID NO: 15 - DNA, 23 bp, Artificial Sequence
  target sequence
gaacctgagctgctctgacgcgg

SEQ ID NO: 16 - DNA, 23 bp, Artificial Sequence
  target sequence
cggccgcgtcagagcagctcagg

SEQ ID NO: 17 - DNA, 23 bp, Artificial Sequence
  target sequence
ctggtgcgtttcactgatcctgg

SEQ ID NO: 18 - DNA, 23 bp, Artificial Sequence
  target sequence
gcttccttacacttcccaagagg

SEQ ID NO: 19 - DNA, 23 bp, Artificial Sequence
  target sequence
gatcagtgaaacgcaccagacgg

SEQ ID NO: 20 - DNA, 23 bp, Artificial Sequence
  target sequence
ttggtcctgagttctaactttgg

SEQ ID NO: 21 - DNA, 23 bp, Artificial Sequence
  target sequence
tcccaagaggagaagcagtttgg

SEQ ID NO: 22 - DNA, 23 bp, Artificial Sequence
  target sequence
cggaggaacaatataaattgggg

SEQ ID NO: 23 - DNA, 23 bp, Artificial Sequence
  target sequence
tcacaggtaaaactgacgcacgg

SEQ ID NO: 24 - DNA, 23 bp, Artificial Sequence
  target sequence
gccagtagccagccccgtcctgg

SEQ ID NO: 25 - DNA, 23 bp, Artificial Sequence
  target sequence
gtagccagccccgtcctggcagg

SEQ ID NO: 26 - DNA, 23 bp, Artificial Sequence
  target sequence
ggctactggccttatctcacagg

SEQ ID NO: 27 - DNA, 23 bp, Artificial Sequence
  target sequence
cctaggtgttcaccaggtcgtgg

SEQ ID NO: 28 - DNA, 23 bp, Artificial Sequence
  target sequence
ttgtgagaatggtgcgtcctagg

SEQ ID NO: 29 - DNA, 23 bp, Artificial Sequence
  target sequence
gagtagaggcggccacgacctgg

SEQ ID NO: 30 - DNA, 23 bp, Artificial Sequence
  target sequence
gtgcgtcctaggtgttcaccagg

SEQ ID NO: 31 - DNA, 23 bp, Artificial Sequence
  target sequence
aaagagtccccagtgctatctgg

SEQ ID NO: 32 - DNA, 19 bp, Artificial Sequence
  TALE binding site, target sequence
tccatcgtaagcaaacctt

SEQ ID NO: 33 - DNA, 19 bp, Artificial Sequence
  TALE binding site, target sequence
tcccaccccctgccaagct

SEQ ID NO: 34 - DNA, 19 bp, Artificial Sequence
  TALE binding site, target sequence
tccaaactgcttctcctct

SEQ ID NO: 35 - DNA, 19 bp, Artificial Sequence
  TALE binding site, target sequence
tccacacggacacccccct

SEQ ID NO: 36 - DNA, 19 bp, Artificial Sequence
  TALE binding site, target sequence
tccaccatctcatgcccct

SEQ ID NO: 37 - DNA, 19 bp, Artificial Sequence
  TALE binding site, target sequence
tctaaggtttgcttacgat

SEQ ID NO: 38 - DNA, 19 bp, Artificial Sequence
  TALE binding site, target sequence
tcctctctggctccatcgt

SEQ ID NO: 39 - DNA, 19 bp, Artificial Sequence
  TALE binding site, target sequence
tggtcctgagttctaactt

SEQ ID NO: 40 - DNA, 19 bp, Artificial Sequence
  TALE binding site, target sequence
tcctccgtgcgtcagtttt

SEQ ID NO: 41 - DNA, 148 bp, Artificial Sequence
  T6-SK SMArT minimal promoter
tctagaattagctttaggcgtgtacggtgggcgcctataaaagcagagctcgtttagtgaaccgtcagatcgcctggagcaattccacaacacttttgtcttataccaactttccgtaccacttcctaccctcgtaaagaatccgcgg

SEQ ID NO: 42 - DNA, 109 bp, Artificial Sequence
  minCMV SMArT minimal promoter
gtacggtgggaggcctatataagcagagctcgtttagtgaaccgtcagatcgcctggagacgccatccacgctgttttgacctccatagaagacaccgggaccgatcca

SEQ ID NO: 43 - DNA, 720 bp, Artificial Sequence
  selector eGFP
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 44 - DNA, 843 bp, Artificial Sequence
  selector NGFR
atgggggcaggtgccaccggccgcgccatggacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgacgacgccgtgtgccgctgcgcctacggctactaccaggatgagacgactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggccttgtggcctacatagccttcaagaggtggaacagggggatcctctag

SEQ ID NO: 45 - DNA, 4284 bp, Artificial Sequence
  engineered transcriptional transactivator (ETT) Sp-dCas9
atgggcggtaggcgtgtacggtgggaggtctatataagcagagctcgtttagtgaaccgtcagatcgcctggagacgccatccacgctgttttgacctccatagaagacaccgggaccgatccagcctccggactctagaggatcgaacccttgccaccatggacaagaagtactccattgggctcgctatcggcacaaacagcgtcggctgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggctgctatcgtgccccagtcttttctcaaagatgattctattgataataaagtgttgacaagatccgataaagctagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagtga

SEQ ID NO: 46 - DNA, 417 bp, Artificial Sequence
  ETT VP160 domain
gatccggaggcggggcggacgcgctggacgatttcgatctcgacatgctgggttctgatgccctcgatgactttgacctggatatgttgggaagcgacgcattggatgactttgatctggacatgctcggctccgatgctctggacgatttcgatctcgatatgttagggtcagacgcactggatgatttcgaccttgatatgttgggaagcgatgcccttgatgatttcgacctggacatgctcggcagcgacgccctggacgatttcgatctggacatgctggggtccgatgccttggatgattttgacttggatatgctggggagtgatgccctggacgactttgacctggacatgctgggctccgatgcgctcgatgacttcgatttggatatgttgtattgaaagcttctga

SEQ ID NO: 47 - DNA, 1677 bp, Artificial Sequence
  ETT VPR domain
aagaagaggaaggtgtcgccagggatccgtcgacttgacgcgttgatatcaacaagtttgtacaaaaaagcaggctacaaagaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactctagaagttccggatctccgaaaaagaaacgcaaagttggtagccagtacctgcccgacaccgacgaccggcaccggatcgaggaaaagcggaagcggacctacgagacattcaagagcatcatgaagaagtcccccttcagcggccccaccgaccctagacctccacctagaagaatcgccgtgcccagcagatccagcgccagcgtgccaaaacctgccccccagccttaccccttcaccagcagcctgagcaccatcaactacgacgagttccctaccatggtgttccccagcggccagatctctcaggcctctgctctggctccagcccctcctcaggtgctgcctcaggctcctgctcctgcaccagctccagccatggtgtctgcactggctcaggcaccagcacccgtgcctgtgctggctcctggacctccacaggctgtggctccaccagcccctaaacctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctgggctctggcagcggcagccgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactggatccagcgcccgcagtgactcccgaggccagtcacctgttggaggatcccgatgaagagacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgttttga

SEQ ID NO: 48 - DNA, 2521 bp, Artificial Sequence
  ETT TALE7 DBD (SMArT)
aaacgggccctctagactcgagcggccgcgccaccatgggaaaacctattcctaatcctctgctgggcctggattctaccggaggcatggcccctaagaaaaagcggaaggtggacggcggagtggacctgagaacactgggatattctcagcagcagcaggagaagatcaagcccaaggtgagatctacagtggcccagcaccacgaagccctggtgggacacggatttacacacgcccacattgtggccctgtctcagcaccctgccgccctgggaacagtggccgtgaaatatcaggatatgattgccgccctgcctgaggccacacacgaagccattgtgggagtgggaaaacagtggtctggagccagagccctggaagccctgctgacagtggccggagaactgagaggacctcctctgcagctggatacaggacagctgctgaagattgccaaaaggggcggagtgaccgcggtggaagccgtgcacgcctggagaaatgccctgacgggtgcccccctgaacctgaccccggaccaagtggtggctatcgccagccacgatggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgaccccggaccaagtggtggctatcgccagcaatcacggcggcaagcaagcgctcgaaacggtgcagcggctgttgccggtgctgtgccaggaccatggcctgaccccggaccaagtggtggctatcgccagcaatcacggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagcaatcacggcggcaagcaagcgctcgaaacggtgcagcggctgttgccggtgctgtgccaggaccatggcctgaccccggaccaagtggtggctatcgccagcaacggtggcggcaagcaagcgctcgaaagcattgtggcccagctgagccggcctgatccggcgttggccgcgttgaccaacgatcacctggtggccctggcctgtctgggaggcagacctgccctggatgccgtgaaaaaaggactgcctcacgcccctgccctgatcaagagaacaaatagaagaatccccgagcggacctctcacagagtggccggatcaca

SEQ ID NO: 49 - DNA, 2391 bp, Artificial Sequence
  ETT TALE3 DBD (SMArTER)
gtggacctgagaacactgggatattctcagcagcagcaggagaagatcaagcccaaggtgagatctacagtggcccagcaccacgaagccctggtgggacacggatttacacacgcccacattgtggccctgtctcagcaccctgccgccctgggaacagtggccgtgaaatatcaggatatgattgccgccctgcctgaggccacacacgaagccattgtgggagtgggaaaacagtggtctggagccagagccctggaagccctgctgacagtggccggagaactgagaggacctcctctgcagctggatacaggacagctgctgaagattgccaaaaggggcggagtgaccgcggtggaagccgtgcacgcctggagaaatgccctgacgggtgcccccctgaacctgaccccggaccaagtggtggctatcgccagcaacaatggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaatcacggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgaccccggaccaagtggtggctatcgccagcaacggtggcggcaagcaagcgctcgaaagcattgtggcccagctgagccggcctgatccggcgttggccgcgttgaccaacgatcacctggtggccctggcctgtctgggaggcagacctgccctggatgccgtgaaaaaaggactgcctcacgcccctgccctgatcaagagaacaaatagaagaatccccgagcggacctctcacagagtggcc

SEQ ID NO: 50 - DNA, 801 bp, Artificial Sequence
  Left homology arm of AAVS1-targeting constructs
ccactgtggggtggaggggacagataaaagtacccagaaccagagccacattaaccggccctgggaatataaggtggtcccagctcggggacacaggatccctggaggcagcaaacatgctgtcctgaagtggacataggggcccgggttggaggaagaagactagctgagctctcggacccctggaagatgccatgacagggggctggaagagctagcacagactagagaggtaaggggggtaggggagctgcccaaatgaaaggagtgagaggtgacccgaatccacaggagaacggggtgtccaggcaaagaaagcaagaggatggagaggtggctaaagccagggagacggggtactttggggttgtccagaaaaacggtgatgatgcaggcctacaagaaggggaggcgggacgcaagggagacatccgtcggagaaggccatcctaagaaacgagagatggcacaggccccagaaggagaaggaaaagggaacccagcgagtgaagacggcatggggttgggtgagggaggagagatgcccggagaggacccagacacggggaggatccgctcagaggacatcacgtggtgcagcgccgagaaggaagtgctccggaaagagcatccttgggcagcaacacagcagagagcaaggggaagagggagtggaggaagacggaacctgaaggaggcggcagggaaggatctgggccagccgtagaggtgacccaggccacaagctgcagacagaaagcggcacaggcccaggggagagaatgctggtcagagaaagca

SEQ ID NO: 51 - DNA, 256 bp, Artificial Sequence
  SV40polyA of AAVS1-targeting constructs
gagatccagacatgataagatacattgatgagtttggacaaaccaaaactagaatgcagtgaaaaaaatgccttatttgtgaaatttgtgatgctattgccttatttgtaaccattataagctgcaataaacaagttaacaacaacaattgcattcattttatgtttcaggttcagggggaggtgtgggaggttttttaaagcaagtaaaacctctacaaatgtggtatggctgattatgatcagatctctcgagg

SEQ ID NO: 52 - DNA, 147 bp, Artificial Sequence
  Shortened left homology arm of AAVS1-targeting constructs
gcaaggagagagatggctccaggaaatgggggtgtgtcaccagataaggaatctgcctaacaggaggtgggggttagacccaatatcaggagactaggaaggaggaggcctaaggatggggcttttctgtcaccaatcctgtcccta

SEQ ID NO: 53 - DNA, 290 bp, Artificial Sequence
  Left homology arm of IL2RG-targeting constructs
agaggaaacgtgtgggtggggaggggtagtgggtgagggacccaggttcctgacacagacagactacacccagggaatgaagagcaagcgccatgttgaagccatcattaccattcacatccctcttattcctgcagctgcccctgctgggagtggggctgaacacgacaattctgacgcccaatgggaatgaagacaccacagctggtgggaaatctgggactggagggggctggtgagaagggtggctgtgggaaggggccgtacagagatctggtgcctgccactgg

SEQ ID NO: 54 - DNA, 1076 bp, Artificial Sequence
  IL2RG recoded corrective cDNA (for intron 1 gene correction strategy) of IL2RG-targeting constructs
atttctttctgaccaccatgcccaccgacagcctgagcgtgagcaccctgcccctgcccgaggtgcagtgcttcgtgttcaacgtggagtacatgaactgcacctggaacagcagcagcgagccccagcccaccaatctgaccctgcactactggtacaagaacagcgacaacgacaaggtgcagaagtgcagccactacctgttcagcgaggaaatcaccagcggctgccagctgcagaagaaagagatccacctgtaccagaccttcgtggtgcagctgcaggacccccgggagccccgcaggcaggccacccagatgctgaagctgcagaacctggtgatcccctgggcccctgagaacctgacactgcacaagctgtccgagagccagctggaactgaactggaacaaccgcttcctgaaccactgcctggaacacctggtgcagtaccggaccgactgggaccacagctggaccgagcagagcgtggactaccggcacaagttcagcctgcccagcgtggacggccagaagcggtacaccttcagagtgcggagccggttcaaccccctgtgcggcagcgcccagcactggtccgagtggagccaccccatccactggggcagcaacaccagcaaagagaaccccttcctgttcgccctggaagccgtggtgatcagcgtgggcagcatgggcctgatcatctccctgctgtgcgtgtacttctggctggaacggaccatgcccagaatccccaccctgaagaacctggaagatctggtgaccgagtaccacggcaacttcagcgcctggtccggcgtgagcaagggcctggccgagagcctgcagcccgactacagcgagcggctgtgcctggtgtccgagatcccccccaaaggcggagccctgggcgaaggccctggcgccagcccctgcaaccagcacagcccctactgggcccctccttgctacaccctgaagcccgagacccgggccaagcgatccggatccggagccaccaacttcagcctgctgaagcaggccggcgacgtggaggagaaccccggcccctga

SEQ ID NO: 55 - DNA, 81 bp, Artificial Sequence
  Furin site + self-cleaving 2A peptide of IL2RG-targeting constructs
cgggccaagcgatccggatccggagccaccaacttcagcctgctgaagcaggccggcgacgtggaggagaaccccggcccc

SEQ ID NO: 56 - DNA, 267 bp, Artificial Sequence
  Right homology arm of IL2RG-targeting constructs
tacaatcatgtgggcagaattgaaaagtggagtgggaagggcaagggggagggttccctgcctcacgctacttcttctttctttcttgtttgtttgtttctttctttcttttgaggcagggtctcactatgttgcctaggctggtctcaaactcctggcctctagtgatcctcctgcctcagcctttcaaagcaccaggattacagacatgagccaccgtgcttggcctcctccttctgaccatcatttctctttccctccctgcct

SEQ ID NO: 57 - DNA, 516 bp, Artificial Sequence
  PGK promoter of IL2RG-targeting constructs
ccacggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcggggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagg

SEQ ID NO: 58 - DNA, 1041 bp, Artificial Sequence
  tTA and 3′ UTR
atgtctagactggacaagagcaaagtcataaactctgctctggaattactcaatgaagtcggtatcgaaggcctgacgacaaggaaactcgctcaaaagctgggagttgagcagcctaccctgtactggcacgtgaagaacaagcgggccctgctcgatgccctggcaatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggatcaggagcatcaagtagcaaaagaggaaagagagacacctaccaccgattctatgcccccacttctgagacaagcaattgagctgttcgaccatcagggagccgaacctgccttccttttcggcctggaactaatcatatgtggcctggagaaacagctaaagtgcgaaagcggcgggccggccgacgcccttgacgattttgacttagacatgctcccagccgatgcccttgacgactttgaccttgatatgctgcctgctgacgctcttgacgattttgaccttgacatgctccccgggtaaaagctcgctttcttgctgtccaatttctattaaaggttcctttgttccctaagtccaactactaaactgggggatattatgaagggccttgagcatctggattctgcctaataaaaaacatttattttcattgctgcgctagaagctcgctttcttgctgtccaatttctattaaaggttcctttgttccctaagtccaactactaaactgggggatattatgaagggccttgagcatctggattctgcctaataaaaaacatttattttcattgctgcgggacattcttaatt

SEQ ID NO: 59 - DNA, 19 bp, Artificial Sequence
  Tet operon (TetO) sequence
tccctatcagtgatagaga

SEQ ID NO: 60 - DNA, 16 bp, Artificial Sequence
  spacer sequence
acgatgtcgagtttac

SEQ ID NO: 61 - DNA, 796 bp, Artificial Sequence
  Left homology arm of AAV6 AAVS1
tgctttctctgaccagcattctctcccctgggcctgtgccgctttctgtctgcagcttgtggcctgggtcacctctacggctggcccagatccttccctgccgcctccttcaggttccgtcttcctccactccctcttccccttgctctctgctgtgttgctgcccaaggatgctctttccggagcacttccttctcggcgctgcaccacgtgatgtcctctgagcggatcctccccgtgtctgggtcctctccgggcatctctcctccctcacccaaccccatgccgtcttcactcgctgggttcccttttccttctccttctggggcctgtgccatctctcgtttcttaggatggccttctccgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctccaccccac

SEQ ID NO: 62 - DNA, 258 bp, Artificial Sequence
  Right homology arm of AAV6 AAVS1
tagggacaggattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtgg

SEQ ID NO: 63 - DNA, 496 bp, Artificial Sequence
  Left homology arm of AAV6 CD40LG
tgctttaaaagtaagctatttttttatggagacagcttttttcttttaaatttccagctaggcaagaagagcgtcaatttgatctaaaatttcataatgcttcagattaacatagacatggataagtcccagaatttgcagtcttttagtaaaagtagcattttctgtgtaattcttcacaagcactgattgtagttgcaggatgctcagtctccctctgagatgttttacatttttaaatggttagacttgcaggaacaaaagagcagagtaacttagtaggctgttttgcattcttaggaaaagaaaaccatcaggacttattttgttttcatgtattttttcacttccactgaggagtataattggctggtgttgacaaaataccaatcatagatgtaaaggagaaagttgattagttttctggctgttcctaaaattctggatgcaggaactgtggctagaaagcatctggatgattgcactttaccttagg

SEQ ID NO: 64 - DNA, 499 bp, Artificial Sequence
  Right homology arm of AAV6 CD40LG
cagggatacttgagtgtcctctcttaggatctggacctagaattaatgtcatgagatttttctaacaggataaggtgaggtagtgagggctgaagtcatccactgggttatccaaatattaggtttcactgctgacaaaagagggggcttctggtctggttggttatttgtgtttggcctgatgtgctctgtcaatcaaatgtatggacataggcctagcttctaaaggggcaatagtgacctcagtggactgatatttaccgtactatttacatgtgctcttaattacagcagaagctgccagctaactgaatcttgttttgaatctaaaaaatctactcttaaagcaagaaaatggtataaaattagttgataatgcaagtgaattctgtacatttaattattctaagacattggaaaataaaatatcttgttactttgaggataaaagatgatttctttaaaaatgcaaatgttttctacaaatactaaagttaaa

SEQ ID NO: 65 - DNA, 371 bp, Artificial Sequence
  TETO7-SK sequence
ccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttaggcgtgtacggtgggcgcctataaaagcagagctcgtttagtgaaccgtcagatcgcctggagcaattccacaacacttttgtcttataccaactttccgtaccacttcctaccctcgtaaag

SEQ ID NO: 66 - DNA, 18 bp, Artificial Sequence
  Tet operon (TetO) sequence
ccctatcagtgatagaga

SEQ ID NO: 67 - DNA, 843 bp, Artificial Sequence
  delta LNGFR codon optimized for human expression
atgggagctggtgctaccggcagagctatggatggacctagactgctgctcctgctgctgctcggagtttctcttggcggagccaaagaggcctgtcctaccggcctgtatacacactctggcgagtgctgcaaggcctgcaatcttggagaaggcgtggcacagccttgcggcgctaatcagacagtgtgcgagccttgcctggacagcgtgacctttagcgacgtggtgtctgccaccgagccatgcaagccttgtaccgagtgtgtgggcctgcagagcatgtctgccccttgtgtggaagccgacgatgccgtgtgtagatgcgcctacggctactaccaggacgagacaacaggcagatgcgaggcctgtagagtgtgtgaagccggctctggactggtgttcagctgccaagacaagcagaacaccgtgtgcgaggaatgccccgatggcacctatagcgacgaggccaaccatgtggatccctgcctgccttgtactgtgtgcgaagataccgagcggcagctgcgcgagtgtacaagatgggctgatgccgagtgcgaagagatccccggcagatggatcaccagaagcacacctccagagggcagcgatagcacagccccttctacacaagagcccgaggctcctcctgagcaggatctgattgcctctacagtggccggcgtggtcacaacagtgatgggatcttctcagcccgtggtcaccagaggcaccaccgacaatctgatccccgtgtactgtagcatcctggccgccgtggttgtgggactcgtggcctatatcgccttcaagcggtggaaccggggcatcctgtaa

SEQ ID NO: 68 - DNA, 1077 bp, Artificial Sequence
  CXCR4 WT sequence
gccaccatgtctattcctctgcccctgctgcagatctacaccagcgacaactacaccgaggaaatgggcagcggcgactacgacagcatgaaggaaccctgcttccgggaagagaacgccaacttcaacaagatcttcctgcccacaatctacagcatcatctttctgaccggcatcgtgggcaacggactcgtgatcctcgtgatgggctaccagaaaaagctgcggagcatgaccgacaagtaccggctgcacctgagcgtggccgacctgctgttcgtgatcaccctgcctttctgggccgtggacgccgtggccaattggtacttcggcaacttcctgtgcaaggccgtgcacgtgatctacacagtgaacctgtacagcagcgtgctgatcctggccttcatcagcctggacagatacctggccatcgtgcacgccaccaacagccagcggcctagaaagctgctggccgagaaggtggtgtacgtgggcgtgtggattcccgccctgctgctgaccatccccgacttcatcttcgccaacgtgtccgaggccgacgaccggtacatctgcgaccggttctaccccaacgacctgtgggtggtggtgttccagttccagcacatcatggtgggactgatcctgcctggcatcgtgattctgagctgctactgcatcatcatcagcaagctgagccacagcaagggccaccagaagcggaaggccctgaaaaccaccgtgatcctgattctggctttcttcgcctgctggctgccctactacatcggcatcagcatcgacagcttcatcctgctggaaatcatcaagcagggctgcgagttcgagaacaccgtgcacaagtggatcagcattaccgaggccctggcctttttccactgctgcctgaaccctatcctgtacgccttcctgggcgccaagttcaagacctctgcccagcacgccctgaccagcgtgtccagaggaagcagcctgaagatcctgagcaagggcaagagaggcggccacagctccgtgtctacagagagcgagagcagcagcttccacagcagctga

SEQ ID NO: 69 - DNA, 1077 bp, Artificial Sequence
  CXCR4 WHIM sequence
gccaccatgtctattcctctgcccctgctgcagatctacaccagcgacaactacaccgaggaaatgggcagcggcgactacgacagcatgaaggaaccctgcttccgggaagagaacgccaacttcaacaagatcttcctgcccacaatctacagcatcatctttctgaccggcatcgtgggcaacggactcgtgatcctcgtgatgggctaccagaaaaagctgcggagcatgaccgacaagtaccggctgcacctgagcgtggccgacctgctgttcgtgatcaccctgcctttctgggccgtggacgccgtggccaattggtacttcggcaacttcctgtgcaaggccgtgcacgtgatctacacagtgaacctgtacagcagcgtgctgatcctggccttcatcagcctggacagatacctggccatcgtgcacgccaccaacagccagcggcctagaaagctgctggccgagaaggtggtgtacgtgggcgtgtggattcccgccctgctgctgaccatccccgacttcatcttcgccaacgtgtccgaggccgacgaccggtacatctgcgaccggttctaccccaacgacctgtgggtggtggtgttccagttccagcacatcatggtgggactgatcctgcctggcatcgtgattctgagctgctactgcatcatcatcagcaagctgagccacagcaagggccaccagaagcggaaggccctgaaaaccaccgtgatcctgattctggctttcttcgcctgctggctgccctactacatcggcatcagcatcgacagcttcatcctgctggaaatcatcaagcagggctgcgagttcgagaacaccgtgcacaagtggatcagcattaccgaggccctggcctttttccactgctgcctgaaccctatcctgtacgccttcctgggcgccaagttcaagacctctgcccagcacgccctgaccagcgtgtccagaggaagcagcctgaagatcctgagcaagggcaagtgaggcggccacagctccgtgtctacagagagcgagagcagcagcttccacagcagctga

SEQ ID NO: 70 - DNA, 744 bp, Artificial Sequence
  reverse-tTA (rtTA)
atgtctagactggacaagagcaaagtcataaactctgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgagacaagcaattgagctgttcgaccggcagggagccgaacctgccttccttttcggcctggaactaatcatatgtggcctggagaaacagctaaagtgcgaaagcggcgggccggccgacgcccttgacgattttgacttagacatgctcccagccgatgcccttgacgactttgaccttgatatgctgcctgctgacgctcttgacgattttgaccttgacatgctccccggg

SEQ ID NO: 71 - DNA, 23 bp, Artificial Sequence
  genomic sequence, Guide ID AAVS1gRNA
gtcaccaatcctgtccctagtgg

SEQ ID NO: 72 - DNA, 23 bp, Artificial Sequence
  genomic sequence, Guide ID IL2RG gRNA
actggccattacaatcatgtggg

SEQ ID NO: 73 - DNA, 23 bp, Artificial Sequence
  genomic sequence, Guide ID CD40LG gRNA
tggatgattgcactttatcaggg

SEQ ID NO: 74 - DNA, 618 bp, Artificial Sequence
  TetR sequence
atgtctagactggacaagagcaaagtcataaactctgctctggaattactcaatgaagtcggtatcgaaggcctgacgacaaggaaactcgctcaaaagctgggagttgagcagcctaccctgtactggcacgtgaagaacaagcgggccctgctcgatgccctggcaatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggatcaggagcatcaagtagcaaaagaggaaagagagacacctaccaccgattctatgcccccacttctgagacaagcaattgagctgttcgaccatcagggagccgaacctgccttccttttcggcctggaactaatcatatgtggcctggagaaacagctaaagtgcgaaagcggc

SEQ ID NO: 75 - DNA, 36 bp, Artificial Sequence
  VP16 sequence
gccgacgcccttgacgattttgacttagacatgctc

SEQ ID NO: 76 - DNA, 18 bp, Artificial Sequence
  TetO sequence
ccctatcagtgatagaga

SEQ ID NO: 77 - DNA, 20 bp, Artificial Sequence
  NHEJ AAVS1 FW primer
cttcaggacagcatgtttgc

SEQ ID NO: 78 - DNA, 20 bp, Artificial Sequence
  NHEJ AAVS1 RV primer
acaggaggtgggggttagac

SEQ ID NO: 79 - DNA, 20 bp, Artificial Sequence
  NHEJ Intron 1 IL2RG FW primer
caccctctgtaaagccctgg

SEQ ID NO: 80 - DNA, 20 bp, Artificial Sequence
  NHEJ Intron 1 IL2RG RV primer
aagaaatctagattggggag

SEQ ID NO: 81 - DNA, 20 bp, Artificial Sequence
  Intron 1 IL2RG 3′ integration junction ddPCR FW primer
ctagattggggagaaaatga

SEQ ID NO: 82 - DNA, 19 bp, Artificial Sequence
  Intron 1 IL2RG 3′ integration junction ddPCR RV primer
gtgggaaggggccgtacag

SEQ ID NO: 83 - DNA, 24 bp, Artificial Sequence
  Intron 1 IL2RG 3′ integration junction ddPCR probe (FAM)
gtagctcctatgctaggcgtagcc

SEQ ID NO: 84 - DNA, 21 bp, Artificial Sequence
  AAVS1 3′ integration junction ddPCR FW (SK) primer
caccgtacacgcctaaagcta

SEQ ID NO: 85 - DNA, 21 bp, Artificial Sequence
  AAVS1 3′ integration junction ddPCR FW (mCMV) primer
cttatataggcctcccaccgt

SEQ ID NO: 86 - DNA, 20 bp, Artificial Sequence
  AAVS1 3′ integration junction ddPCR RV primer
tcttgggaagtgtaaggaag

SEQ ID NO: 87 - DNA, 20 bp, Artificial Sequence
  AAVS1 3′ integration junction ddPCR probe (FAM)
ccagataaggaatctgccta

SEQ ID NO: 88 - DNA, 20 bp, Artificial Sequence
  AAVS1 3′ integration junction ddPCR FW primer
gattgggaagacaatagcag

SEQ ID NO: 89 - DNA, 20 bp, Artificial Sequence
  CD40LG 3′ integration junction ddPCR FW primer
ttaggagggggtctgataca

SEQ ID NO: 90 - DNA, 25 bp, Artificial Sequence
  CD40LG 3′ integration junction ddPCR RV primer
tcctcgatctgtgggaggaagagaa

SEQ ID NO: 91 - DNA, 20 bp, Artificial Sequence
  CD40LG 3′ integration junction ddPCR Probe (FAM)
tcagtctccctctgagatgt

(19) United States
(12) Patent Application Publication
(10) Pub. No.: US 2022/0056484 A1
(43) Pub. Date: Feb. 24, 2022

(54) SELECTION BY MEANS OF ARTIFICIAL TRANSACTIVATORS

(71) Applicants: Ospedale San Raffaele S.R.L., Milan (IT); Fondazione Telethon, Rome (IT)

(72) Inventors: Pietro Genovese, Milan (IT); Samuele Ferrari, Milan (IT); Angelo Leone Lombardo, Milan (IT); Luigi Naldini, Milan (IT); Martina Fiumara, Milan (IT)

(21) Appl. No.: 17/284,160

(22) PCT Filed: Oct. 11, 2019
(86) PCT No.: PCT/EP2019/077657

§ 371 Date: Apr. 9, 2021

(30) Foreign Application Priority Data
Oct. 11, 2018 (EP) 18199952.5

(51) Int. Cl. C12N 15/90; A61K 35/28; A61K 38/46; A61K 48/00; C12N 15/86

(52) U.S. Cl. CPC C12N 15/907; A61K 35/28; A61K 38/465; C12N 2750/14143; C12N 15/86; C07K 2319/71; C07K 2319/80; A61K 48/005

(57) ABSTRACT

A method for selecting genome edited cells and/or for enrichment of genome edited cells in a population of cells comprising: (a) introducing into a cell or a population of cells at least one first component, at least one second component and at least one third component; and (b) selecting the genome edited cells which transiently express or transiently upregulate a nucleotide sequence encoding a selector.

### CROSS-REFERENCE TO RELATED APPLICATIONS

This application is a U.S. National Phase of International Patent Application No. PCT/EP2019/077657, filed on Oct. 11, 2019, which claims priority to European Patent Application No. 18199952.5, filed on Oct. 11, 2018.

### INCORPORATION BY REFERENCE OF MATERIAL SUBMITTED ELECTRONICALLY

A Sequence Listing is incorporated herein by reference as part of the disclosure. The sequence listing has been submitted as a text file named “56486_Seqlisting”, which was created on Apr. 8, 2021, and is 49,234 bytes in size.

### FIELD

The present invention relates to methods for selecting genome edited cells and/or for the enrichment of genome edited cells in a population of cells. The present invention also relates to a population of genome edited cells produced by the method, pharmaceutical compositions comprising the population of genome edited cells, use of the population of genome edited cells for therapy (such as gene therapy, hematopoietic stem cell transplantations, cancer treatments and tissue repair), and methods for the treatment or prevention of disorders, such as X-linked SCID and skin diseases or retinal diseases, comprising the step of administering a population of genome edited cells.

### BACKGROUND

Hematopoietic stem cell (HSC) gene therapy has provided substantial therapeutic benefit in patients affected by several hematological and non-hematological diseases. Yet, the use of semi-randomly integrating vectors still poses the risk of insertional mutagenesis and non-physiological transgene expression. Therefore, the scope of HSC genetic engineering has broadened from gene replacement to targeted genome editing, which relies on the use of artificial nucleases for precise modification of endogenous genes. Gene editing in primitive hematopoietic stem/progenitor cells (HSPCs) has remained elusive for years. Indeed, the induction of DNA double strand break (DSB) in these cells may trigger apoptosis, differentiation or senescence and its repair may proceed by non-homologous end joining (NHEJ) rather than homology-directed DNA repair (HDR), which requires upregulation of the relevant protein machinery and progression of the targeted cell through the cell cycle.

Remarkable advances in ex vivo HSC culturing, nuclease design and gene transfer technologies has allowed these barriers to be partially overcome and has provided clear evidence of targeted genome editing in human HSPCs capable of long-term multi-lineage reconstitution in immunodeficient NSG mice (Genovese et al., 2014). More recently, further optimizations of gene editing reagents and procedures significantly improved gene targeting efficiency in primitive HSPCs, achieving on average 10-20% of gene marking in long-term engrafting HSPCs (De Ravin et al., 2017; Dever et al., 2016; Wang et al., 2015). These levels of gene correction have been predicted to be safe and effective for the treatment of some diseases, such as in the case of X-linked Severe Combined Immunodeficiency (SCID-X1), in which the edited progeny would have the benefit of selective advantage over non-edited counterpart (Schiroli et al., 2017).

However, there are several challenges that HSC gene editing faces before it becomes suitable for broader clinical application. In particular, it is crucial to improve the efficiency of gene editing in more primitive HSPCs as well as the yield of edited HSCs. Although further improvements in gene editing protocols might enhance gene targeting efficiency, in vitro selection of corrected HSPCs before administration could be a valuable strategy to obtain a pure population of edited cells. Selection of the gene edited cells is difficult when the level of gene correction is not sufficient to achieve the reversion of a pathological process. Further, it may be difficult to directly select for the intended editing as the corrected gene may not provide a growth advantage (such as occurs in chronic granulomatous disease, thalassemias, RAG1 deficiency, etc). In addition, the gene edited cells may not be amenable to selection because of intracellular localization of its relative protein product or the protein may not even be expressed in HSPCs.

To overcome these hurdles, gene editing might be coupled with constitutive expression of selectable markers, such as fluorescent proteins, which are amenable for FACS-sorting, or drug-resistance proteins, followed by drug treatment to mediate selection. However, constitutive expression of a reporter gene can be immunogenic or detrimental to long term cell viability, thus potentially leading to the loss of the modified cells. Finally, these systems will lead to the selection of both in situ and off-site targeted insertions, thus jeopardizing the therapeutic and safety outcomes.

The breakthrough of engineered transcriptional transactivators (ETTs) has opened new possibilities to precisely regulate the expression of an intended gene. In particular, TALE and CRISPR/Cas systems have been engineered by fusing the DNA binding domain (DBD) or catalytically inactive Cas9 (dCas9), respectively, with single or multiple transcriptional activator domains (such as VP64 and VPR) (Chavez et al., 2015).

Guo et al (2017, Protein Cell 8(5):379-393) discuss a system to upregulate endogenous genes in human pluripotent stem cells (HPSCs). Guo et al (2017) disclose the insertion of a doxycycline (Dox) inducible dCas9-VP64-p65-Rta and a Tet transactivator expression cassette into two alleles of the AAVS1 locus and that the level of dCas9-VPR can be controlled precisely and reversibly by the addition and withdrawal of Dox.

Dever et al (2016, Nature 539(7629): 384-389) discuss homologous recombination at the HBB gene in human stem cells (HSC) by using the combination of Cas9 ribonucleoproteins and recombinant AAV6 homologous recombination donor delivery. Dever et al (2016) disclose AAV vector plasmids comprising tNGFR. Dever et al (2016) discuss using tNGFR as a marker to enrich HBB targeted HSPCs and anti-NGFR magnetic-bead separation.

Bak et al (2018, Nat Protoc 13(2):358-376) discuss the use of CRISPR/Cas9 technology and recombinant AAV6 homologous donor delivery for editing human hematopoietic stem cells by homologous recombination. Bak et al (2018) disclose reporter genes such as GFP or truncated growth factor receptor (tNGFR). Bak et al (2018) discuss flow cytometry to enrich for cells with targeted integration.

US2015/0191744 discusses methods for modifying the transcriptional regulation of stem or progenitor cells using a lentiviral vector encoding a nuclease deficient Cas9 effector domain fusion protein and a lentiviral vector comprising at least one sgRNA gene complementary to a genomic target. US2015/0191744 discloses a Cas9-fluorescent protein fusion protein.

The present inventors have developed a platform to achieve gene editing and enrichment of edited cells before in vivo administration, thus increasing the efficacy of gene therapy, and potentially expanding its application to a wide spectrum of genetic diseases, including those in which no proliferative growth advantage is conferred to corrected cells.

### SUMMARY

The present inventors have developed methods for selecting edited cells that couple particular donor DNA vector designs (the first component) with the use of engineered transcriptional transactivators (ETTs) (the second component) combined with a nuclease system (the third component) to drive the selection of edited cells. The present inventors have exploited transient expression of neutral genes as selectable markers, such as the cell-surface-expressed mutated Low-affinity Nerve Growth Factor Receptor (ΔLNGFR), upon on-site recombination, and specific selection of edited cells.

The present invention provides a method for selecting genome edited cells and/or for the enrichment of genome edited cells in a population of cells comprising:

(a) introducing into a cell (i.e. the starting cell) or a population of cells (i.e. a starting population of cells) at least one first component, at least one second component and at least one third component; and

(b) selecting the genome edited cells which transiently express or transiently upregulate a nucleotide sequence encoding a selector;

wherein the first component is a donor reporter cassette comprising the nucleotide sequence encoding the selector and a nucleotide sequence of interest (NOI);

wherein the second component is an engineered transcriptional transactivator (ETT) polypeptide or a nucleotide sequence encoding an ETT polypeptide; wherein the ETT polypeptide comprises a DNA binding domain (DBD) and at least one transcription activator (TA) domain;

wherein the third component is a nuclease system comprising a genome targeted nuclease and, optionally, a guide RNA (gRNA) comprising at least one targeted genomic sequence;

wherein the ETT polypeptide is transiently present in the cell or population of cells or the nucleotide sequence encoding the ETT polypeptide is transiently expressed in the cell or population of cells; and

wherein the presence of the nuclease system in the cell or the population of cells enables the insertion of the nucleotide sequence encoding the selector (and, optionally, a minimal promoter) and the NOI into a target locus and wherein the transient presence of the ETT polypeptide or the transient expression of the nucleotide sequence encoding the ETT polypeptide enables transient expression or transient upregulation of the inserted nucleotide sequence encoding the selector.

Transient expression or transient upregulation of a nucleotide sequence encoding a selector enables the specific selection of cells in which the nucleotide sequence encoding the selector and a nucleotide sequence of interest (NOI) has been inserted into the target locus from those cells in which the nucleotide sequence encoding the selector and a nucleotide sequence of interest (NOI) have not been inserted into the target locus.

The present invention provides a method for selecting genome edited cells and/or for the enrichment of genome edited cells in a population of cells comprising:
(a) introducing into a cell or a population of cells at least one first component, at least one second component and at least one third component; and
(b) selecting the genome edited cells which transiently express or transiently upregulate a nucleotide sequence encoding a selector;
wherein the first component is a donor reporter cassette comprising the nucleotide sequence encoding the selector and a nucleotide sequence of interest (NOI) and, optionally, a minimal promoter operably linked to a regulatory element;
wherein the second component is an engineered transcriptional transactivator (ETT) polypeptide or a nucleotide sequence encoding an ETT polypeptide; wherein the ETT polypeptide comprises a DNA binding domain (DBD) and at least one transcription activator (TA) domain;
wherein the third component is a nuclease system comprising a genome targeted nuclease and, optionally, a guide RNA (g RNA) comprising at least one targeted genomic sequence;
wherein the ETT polypeptide is transiently present in the cell or population of cells or the nucleotide sequence encoding the ETT polypeptide is transiently expressed in the cell or population of cells; and
wherein the presence of the nuclease system in the cell or the population of cells enables the insertion of the nucleotide sequence encoding the selector and the NOI and, optionally, the minimal promoter operably linked to the regulatory element into a target locus and wherein the transient presence of the ETT polypeptide or the transient expression of the nucleotide sequence encoding the ETT polypeptide enables transient expression or transient upregulation of the inserted nucleotide sequence encoding the selector optionally when a modulator is present in the cell or population of cells or optionally when a modulator is not present in the cell or population of cells.

The present invention provides a method for selecting genome edited cells and/or for the enrichment of genome edited cells in a population of cells comprising:
(a) introducing into a cell or a population of cells at least one first component, at least one second component and at least one third component; and
(b) selecting the genome edited cells which transiently express or transiently upregulate a nucleotide sequence encoding a selector;
wherein the first component is a donor reporter cassette comprising the nucleotide sequence encoding the selector and a nucleotide sequence of interest (NOI) and a minimal promoter operably linked to a regulatory element;
wherein the second component is an engineered transcriptional transactivator (ETT) polypeptide or a nucleotide sequence encoding an ETT polypeptide; wherein the ETT polypeptide comprises a DNA binding domain (DBD) and at least one transcription activator (TA) domain;
wherein the third component is a nuclease system comprising a genome targeted nuclease and, optionally, a guide RNA (gRNA) comprising at least one targeted genomic sequence;
wherein the ETT polypeptide is transiently present in the cell or population of cells or the nucleotide sequence encoding the ETT polypeptide is transiently expressed in the cell or population of cells, and
the presence of the nuclease system in the cell or the population of cells enables the insertion of the nucleotide sequence encoding the selector, the NOI and the minimal promoter operably linked to the regulatory element into a target locus and wherein the transient presence of the ETT polypeptide or the transient expression of the nucleotide sequence encoding the ETT polypeptide enables transient expression or transient upregulation of the inserted nucleotide sequence encoding the selector when a modulator is present in the cell or population of cells.

The present invention provides a method for selecting genome edited cells and/or for the enrichment of genome edited cells in a population of cells comprising:
(a) introducing into a cell or a population of cells at least one first component, at least one second component and at least one third component; and
(b) selecting the genome edited cells which transiently express or transiently upregulate a nucleotide sequence encoding a selector;
wherein the first component is a donor reporter cassette comprising the nucleotide sequence encoding the selector and a nucleotide sequence of interest (NOI) and a minimal promoter operably linked to a regulatory element;
wherein the second component is an engineered transcriptional transactivator (ETT) polypeptide or a nucleotide sequence encoding an ETT polypeptide; wherein the ETT polypeptide comprises a DNA binding domain (DBD) and at least one transcription activator (TA) domain;
wherein the third component is a nuclease system comprising a genome targeted nuclease and, optionally, a guide RNA (gRNA) comprising at least one targeted genomic sequence;
wherein the ETT polypeptide is transiently present in the cell or population of cells or the nucleotide sequence encoding the ETT polypeptide is transiently expressed in the cell or population of cells, and
the presence of the nuclease system in the cell or the population of cells enables the insertion of the nucleotide sequence encoding the selector, the NOI and the minimal promoter operably linked to the regulatory element into a target locus and wherein the transient presence of the ETT polypeptide or the transient expression of the nucleotide sequence encoding the ETT polypeptide enables transient expression or transient upregulation of the inserted nucleotide sequence encoding the selector when a modulator is not present in the cell or population of cells.

The present invention provides a method for selecting genome edited cells and/or for the enrichment of genome edited cells in a population of cells comprising:
(a) introducing into a cell or a population of cells at least one first component, at least one second component and at least one third component; and
(b) selecting the genome edited cells which transiently express or transiently upregulate a nucleotide sequence encoding a selector;
wherein the first component is a donor reporter cassette comprising the nucleotide sequence encoding the selector and a nucleotide sequence of interest (NOI) and a minimal promoter operably linked to a regulatory element;
wherein the second component is an engineered transcriptional transactivator (ETT) polypeptide or a nucleotide sequence encoding an ETT polypeptide; wherein the ETT polypeptide comprises a DNA binding domain (DBD) and at least one transcription activator (TA) domain;
wherein the third component is a nuclease system comprising a genome targeted nuclease and, optionally, a guide RNA (gRNA) comprising at least one targeted genomic sequence;
wherein the presence of the nuclease system in the cell or the population of cells enables the insertion of the nucleotide sequence encoding the selector, the NOI and the minimal promoter operably linked to the regulatory element into a target locus and wherein the transient presence of a modulator in the cell or population of cells enables transient expression or transient upregulation of the inserted nucleotide sequence encoding the selector when a modulator is present in the cell or population of cells.

The present invention provides a method for selecting genome edited cells and/or for the enrichment of genome edited cells in a population of cells comprising:
(a) introducing into a cell or a population of cells at least one first component, at least one second component and at least one third component; and
(b) selecting the genome edited cells which transiently express or transiently upregulate a nucleotide sequence encoding a selector;
wherein the first component is a donor reporter cassette comprising the nucleotide sequence encoding the selector and a nucleotide sequence of interest (NOI) and a minimal promoter operably linked to a regulatory element;
wherein the second component is an engineered transcriptional transactivator (ETT) polypeptide or a nucleotide sequence encoding an ETT polypeptide; wherein the ETT polypeptide comprises a DNA binding domain (DBD) and at least one transcription activator (TA) domain;
wherein the third component is a nuclease system comprising a genome targeted nuclease and, optionally, a guide RNA (gRNA) comprising at least one targeted genomic sequence;
wherein the presence of the nuclease system in the cell or the population of cells enables the insertion of the nucleotide sequence encoding the selector, the NOI and the minimal promoter operably linked to the regulatory element into a target locus and wherein the transient presence of a modulator in the cell or population of cells enables transient expression or transient upregulation of the inserted nucleotide sequence encoding the selector when the modulator is not present in the cell or population of cells.

Without wishing to be bound by theory, the transient presence of the modulator in the cell or population of cells may occur by the addition of the modulator to the media followed by subsequent washing of the cell or population of cells.

In one embodiment, (herein referred to as Selection by Means of Artificial Transactivators [SMArT]), the first component comprises a donor reporter cassette, in which the expression of a selector (such as the mutated Low-affinity Nerve Growth Factor Receptor [ΔLNGFR]) is driven by a minimal promoter (such as cytomegalovirus [CMV] or synthetic promoter [T6-SK] (Loew, Heinz, Hampf, Bujard, & Gossen, 2010)), that should putatively limit basal expression of the selector. The first component comprises homologous arms (HAs) comprising nucleotide sequences homologous to the target locus, these HAs enable the donor reporter cassette to be inserted into the target locus by homology-driven repair (HDR). The donor reporter cassette further comprises a NOI operably linked to a promoter. ETT binding sites are outside the homology arms and upstream of the minimal promoter. Insulator elements might be also present in the cassette in order to limit the possible enhancer activity of the fully efficient promoter on the minimal promoter. In this case, insulator elements might be derived from CTCF-dependent or independent binding sites (Phillips-Cremins & Corces, 2013) (FIG. 1A). This selection strategy is useful for targeted integration of a transgene expression cassette into a neutral region of the genome (such as the Adeno-Associated Virus Integration Site 1 [AAVS1]), where sustained therapeutic transgene expression can be achieved without perturbing the neighbor gene regulation and the epigenetic landscape (Lombardo et al., 2011). Other possible neutral regions of the genome can be common integration sites (CIS) of lentiviral vectors (such as those identified by Biffi et al., 2013) or other regions possibly defined in the future as “safe harbors” or neutral areas of the genome.

In one embodiment, (herein referred to as Selection by Means of Artificial Transactivators [SMArT] gene correction), the first component comprises a donor reporter cassette, in which the expression of a selector (such as the mutated Low-affinity Nerve Growth Factor Receptor [ΔLNGFR]) is driven by a minimal promoter (such as cytomegalovirus [CMV] or synthetic promoter [T6-SK] (Loew, Heinz, Hampf, Bujard, & Gossen, 2010)), that should putatively limit basal expression of the selector. The first component comprises homologous arms (HAs) comprising nucleotide sequences homologous to the target locus, these HAs enable the donor reporter cassette to be inserted into the target locus by homology-driven repair (HDR). The donor reporter cassette further comprises a NOI. (Optionally, the cassette further comprises a splicing acceptor site and/or a nucleotide sequence encoding a self-cleaving 2A peptide or, alternatively, an internal ribosome entry site (IRES) element.) The insertion of the donor reporter cassette into the target locus corrects a genetic defect and/or introduces a new function into the endogenous gene. ETT binding sites are outside the homology arms and downstream of the minimal promoter. Insulator elements might be also present in the construct in order to limit the possible enhancer activity of the fully efficient promoter on the minimal promoter. In this case, insulator elements might be derived from CTCF-dependent or independent binding sites (Phillips-Cremins & Corces, 2013) (FIG. 1A). This selection strategy is useful for targeted integration of a transgene expression cassette into a neutral region of the genome (such as the Adeno-Associated Virus Integration Site 1 [AAVS1]), where sustained therapeutic transgene expression can be achieved without perturbing the neighbor gene regulation and the epigenetic landscape (Lombardo et al., 2011). Other possible neutral regions of the genome can be common integration sites (CIS) of lentiviral vectors (such as those identified by Biffi et al., 2013) or other regions possibly defined in the future as “safe harbors” or neutral areas of the genome. In gene correction a full or partial wild-type sequence is used to correct a genetic defect or introduce a new function into an endogenous gene (Schiroli et al., 2017).

In another embodiment, (herein referred to as Selection by Means of Artificial Transactivation of Endogenous Receptors [SMArTER]) the donor reporter cassette comprises: (i) a nucleotide sequence of interest; ii) optionally a splicing acceptor site and/or a nucleotide sequence encoding a self-cleaving 2A peptide or, alternatively, an internal ribosome entry site (IRES) element; iii) the cDNA encoding for the selector protein (such as ΔLNGFR), optionally the nucleotide sequence encoding the selector is operably linked to a minimal promoter; and iv) homology arms (HAs) for the intended target site(s). ETT binding sites are situated close to the promoter of the endogenous gene in order to transiently boost (i.e. transiently upregulate) the expression of the selector (and the edited gene) upon targeted integration (FIG. 5A). This strategy can be applied to select cells that have undergone correction of specific genes. In this case, basal expression of the selector protein is dependent from the transcriptional regulation of the edited gene. To reduce the risk of constitutive expression of the selector, the selector can be fused (in frame) with destabilizer domains (DDs), which are able to induce proteasomal degradation of the selector in absence of specific stabilizer ligands (Rakhit, Navarro, & Wandless, 2014). In particular, destabilizer domains can be based on the FKBP domain (Banaszynski et al., 2006), bacterial DHFR protein (Iwamoto, Björklund, Lundberg, Kirik, & Wandless, 2010) or from the Human Estrogen Receptor (Miyazaki et al., 2012; PMID: 22332638; Journal of the American Chemical Society 134(9):3942-3945). This strategy allows the use of biological selectors that specifically boost growth or engraftment of HDR-edited cells. Constitutive and prolonged expression of proteins enhancing homing and engraftment capacity of corrected cells might lead to undesired and exacerbate expansion of edited clones. Coupling ETT-mediated transactivation and DD-based post-translational regulation would allow transient and temporary overexpression of the biological selector (e.g. CXCR4, CD47).

In another embodiment, (herein referred to as Selection by Means of Artificial Transactivation with Doxycycline regulation [SMArT-D]) the first component comprises a donor reporter cassette, in which the expression of a selector (such as GFP) is driven by a minimal promoter (such as synthetic promoter [T6-SK] (Loew, Heinz, Hampf, Bujard, & Gossen, 2010)), that should putatively limit basal expression of the selector, wherein the minimal promoter is operably linked to a regulatory element (such as a tetracycline operator (TetO) sequence). The regulatory element is typically upstream of the minimal promoter. ETT binding sites are within the regulatory element. Without wishing to be bound by theory, in the Tet-Off system, the ETT binds to the regulatory element and activates the minimal promoter when the selector is inserted into the target locus; the binding of a modulator (such as tetracycline or deoxycycline) prevents the binding of the ETT to the regulatory element when a donor reporter cassette has not been inserted into the target locus. Without wishing to be bound by theory, in the Tet-On system, the ETT binds to the regulatory element and activates the minimal promoter when the selector is inserted into the target locus and when a modulator (such as tetracycline or deoxycycline) is present; the binding of a modulator (such as tetracycline or deoxycycline) enables the binding of the ETT to the regulatory element when a donor reporter cassette has been inserted into the target locus. The first component further comprises homologous arms (HAs) comprising nucleotide sequences homologous to the target locus, these HAs enable the donor reporter cassette to be inserted into the target locus by homology-driven repair (HDR). Insulator elements might be also present in the cassette in order to limit the possible enhancer activity of the fully efficient promoter on the minimal promoter. In this case, insulator elements might be derived from CTCF-dependent or independent binding sites (Phillips-Cremins & Corces, 2013) (FIG. 1A). This selection strategy is useful for targeted integration of a transgene expression cassette into a neutral region of the genome (such as the Adeno-Associated Virus Integration Site 1 [AAVS1]), where sustained therapeutic transgene expression can be achieved without perturbing the neighbor gene regulation and the epigenetic landscape (Lombardo et al., 2011). Other possible neutral regions of the genome can be common integration sites (CIS) of lentiviral vectors (such as those identified by Biffi et al., 2013) or other regions possibly defined in the future as “safe harbors” or neutral areas of the genome.

In one aspect, the present invention provides a population of genome edited cells produced by the method according to the present invention.

The population of genome edited cells produced by the method according to the present invention comprise the nucleotide sequence encoding the selector. The nucleotide sequence encoding the selector is transiently expressed such that the nucleotide sequence encoding the selector is no longer expressed after the cells have been selected.

The population of genome edited cells produced by the method according to the present invention is enriched for cells which are capable of expressing the nucleotide of interest (NOI).

In a further aspect, the present invention provides a pharmaceutical composition comprising the population of genome edited cells according to the present invention.

In a broad aspect, the present invention provides a population of genome edited cells according to the present invention for use in therapy.

In another aspect, the present invention provides a population of genome edited cells according to the present invention for use in gene therapy, hematopoietic stem cell transplantation, cancer treatment and/or tissue repair.

The present invention provides, in a further aspect, a population of genome edited cells according to the present invention for use in the treatment or prevention of X-linked Severe Combined Immunodeficiency (SCID-X1), a skin disease and/or a retinal disease.

In a further aspect, the present invention provides a population of genome edited cells according to the present invention for use in the treatment or prevention of a monogeneic disorder such as epidermolysis bullosa and/or retinitis pigmentosa and/or Hyper-IgM (HIGM) syndrome.

In a further aspect, the present invention provides a population of genome edited cells according to the present invention for use in the treatment or prevention of Hyper-IgM (HIGM) syndrome.

In a broad aspect, the present invention provides use of a population of genome edited cells according to the present invention for therapy.

The present invention provides in another aspect, use of a population of genome edited cells according to the present invention for gene therapy, hematopoietic stem cell transplantation, cancer treatment and/or tissue repair.

In a further aspect, the present invention provides use of a population of genome edited cells according to the present invention for the manufacture of a medicament for the treatment or prevention of X-linked SCID, a skin disease and/or a retinal disease.

In a further aspect, the present invention provides use of a population of genome edited cells according to the present invention for the manufacture of a medicament for the treatment or prevention of a monogeneic disorder such as epidermolysis bullosa and/or retinitis pigmentosa and/or Hyper-IgM (HIGM) syndrome.

In a further aspect, the present invention provides use of a population of genome edited cells according to the present invention for the manufacture of a medicament for the treatment or prevention of Hyper-IgM (HIGM) syndrome,

In a broad aspect, the present invention provides a method of therapy comprising the step of administering a population of genome edited cells according to the present invention to a subject.

In another aspect, the present invention provides a method of gene therapy, hematopoietic stem cell transplantation, cancer treatment and/or tissue repair comprising the step of administering a population of genome edited cells according to the present invention to a subject.

The present invention provides, in another aspect, a method for the treatment or prevention of X-linked SCID, a skin disease and/or a retinal disease comprising the step of administering a population of genome edited cells according to the present invention to a subject wherein the subject has X-linked SCID, a skin disease and/or a retinal disease.

The present invention provides, in another aspect, a method for the treatment or prevention of a monogeneic disorder such as epidermolysis bullosa and/or retinitis pigmentosa and/or Hyper-IgM (HIGM) syndrome comprising the step of administering a population of genome edited cells according to the present invention to a subject wherein the subject has a mongeneic disorder.

The present invention provides, in another aspect, a method for the treatment or prevention of Hyper-IgM (HIGM) syndrome comprising the step of administering a population of genome edited cells according to the present invention to a subject wherein the subject has Hyper-IgM (HIGM) syndrome.

### BRIEF DESCRIPTION OF THE FIGURES

FIG. 1. Transient transactivation of a reporter gene stably integrated in the AAVS1 locus by means of engineered transcriptional transactivators. A) FIG. 1Ai: description of SMArT strategy to enrich for AAVS1-edited cells; FIG. 1Aii: description of SMArTER strategy for IL2RG-edited cells; FIG. 1Aiii: description of SMArT strategy for gene correction. B) Isolation and characterization of a single cell-derived clone harboring minimally expressing ΔLNGFR cassette in AAVS1 locus. (Top left) Experimental workflow for clone isolation and characterization. (Bottom left) Molecular analyses to screen for clones with intact 5′ donor-genome junction (5′-TI). Clones positive for 5′-TI were successively screened for intact 3′ donor-genome junction and mono-allelic integration (amplification of the wild-type allele) (middle) and basal expression of ΔLNGFR. Clone H1 was selected for further experiments. (Right) Flow cytometry of the selected clone (clone H1). C) Percentage of NHEJ-mediated editing with gRNAs spanning from 100 to 600 bp from the minimal promoter transcriptional start site (TSS). D) (Top) Schematic of the panel of sgRNAs (single guide RNAs) tested for reporter gene transactivation in K562 clone H. (Bottom) Representative FACS plot of transactivated clone H cells 24 hours upon electroporation. E) Percentage (top) and relative fluorescence intensity (RFI) on untreated clone H cells (UT) (bottom) of ΔLNGFR+ cells at 1, 2 and 14 days after electroporation of plasmid expressing either dCas9-VP160 or dCas9-VPR and selected gRNAs from A. sgRNAs #4, 5, 9, 10, 12, 13, 26, 28 from C has been selected from further transactivation experiments. sgRNAs are ordered from the closest to the farthest from the TSS. The last treatment of each panel of sgRNA is the combination of the first and fifth sgRNAs, respectively. F) (Top) Schematic of the panel of TALE-TA tested for reporter gene transactivation in K562 clone H. Percentage (left) and mean fluorescence intensity (MFI) (right) of ΔLNGFR+ cells at 2, 4 and 12 days after electroporation of plasmid expressing different TALE-VP160 spanning from 324 to 940 bp from the minimal promoter TSS. G) Percentage (top left) and mean fluorescence intensity (MFI) (bottom left) of ΔLNGFR+ cells at 16, 24, 48 and 72 hours after electroporation of increasing doses of TALE#7-VP160 mRNA. (Right) Representative FACS plot from (left).

FIG. 2. SMArT strategy allows to enrich for on target AAVS1-edited cells. A) Optimization of left homology arm (L-HA) length to avoid ETT-mediated transactivation of the not integrated donor. (Top) Construct used to measure by flow cytometry the efficiency of targeted integration. (Bottom) Percentage of GFP+ cells measured 15 days after electroporation of the donor plasmid with or without AAVS1 nucleases. ZFN in FIG. 2A refers to zinc finger nucleases. B) (Bottom) FACS plot representing the basal ΔLNGFR expression upon targeted integration in the AAVS1 locus of constructs with different minimal promoters (minimal CMV or T6-SK) or promoter-less 15 days after electroporation. (Top) Copies per genome of AAVS1-edited alleles measured by digital droplet PCR. C) Percentage (page 10 of the Figures) and MFI (page 11 of the Figures) of highly expressing ΔLNGFR+ cells at 1, 2 and 14 days after electroporation of plasmid expressing either dCas9-VP160 or dCas9-VPR and selected gRNAs (from FIG. 1C) in SK (page 15 of the Figures) or minCMV (page 16 of the Figures) bulk edited population from B. sgRNAs are ordered from the closest to the farthest from the TSS. The last treatment of each panel of sgRNA is the combination of the first and fifth sgRNAs. D) (Top) Schematic for the SMArT proof-of-concept experimental study. (Bottom) Molecular characterization of single cell clones sorted from highly expressing SK-NGFR cells 48 hours upon TALE#7-VP160 plasmid electroporation.

FIG. 3. SMArT strategy in human CB-derived CD34+ cells. A) Experimental procedure for SMArT strategy application in human CD34+ cells. B) (Top) Percentage (left) and relative fluorescence intensity (RFI) on edited only control (D+R) (right) of GFP+ cells at 24 hours after gene targeting and transactivation procedure among HSPC subpopulations. (Bottom) Representative FACS plot showing GFP+ cells within bulk CD34+ HSPC and the most primitive subpopulation (CD90+ cells) in presence or not of TALE#7-VPR-encoding (T7VPR) mRNA (right and left, respectively). C) Percentage of GFP+ cells (left y axis, left-hand column (FACS)) measured by flow cytometry (FACS) and number of copies of edited alleles per cell (right y axis, right-hand column (HDR)) measured by ddPCR in bulk CD34+ culture and among the different HSPC subpopulations. D) (Top) Experimental procedure for SMArT strategy application in human CD34+ cells. (Bottom) Percentage (left) and relative fluorescence intensity (RFI) on edited only control (D+R) (right) of GFP+ cells at 24 hours after electroporation performed one week after thawing among HSPC subpopulations. (D=AAV6 donor transduction; R=AAVS1 RNP; T7VPR=TALE#7VPR-encoding mRNA).

FIG. 4. SMArTER strategy validation in primary human cells. A) Description of SMArTER strategy to enrich for IL2RG-edited cells. B) Fold increase of IL2RG expression in K562 cell lines electroporated with different TALE-VP160-expressing plasmid targeting the promoter of the gene. IL2RG expression has been normalized by using HPRT housekeeping gene and fold increase calculated on mock electroporated control. Steady state expression of (L2RG gene in HEK293T cell line and male B-lymphoblastoid cell line (JY) is also reported as controls. The cell line HEK293T is referred to as 293T. C) Percentage (left) and relative fluorescence intensity (RFI) on edited only control (GT) (right) of GFP+ cells at 24 hours after gene targeting and transactivation procedure among HSPC subpopulations. D) Representative FACS plot showing GFP+ cells within bulk CD34+ HSPC and the most primitive subpopulation (CD90+ cells) in presence or not of TALE#3-VPR-encoding mRNA (bottom and top, respectively). (GT=gene targeting procedure; VP160=TALE#3-VP160-encoding mRNA; VPR=TALE#3-VPR-encoding mRNA).

FIG. 5. SMArTER strategy allows to enrich for IL2RG-corrected HSPCs by boosting the expression of a clinically-compliant selector. A) (Top left) Percentage of male primary T cells edited by HDR with IL2RGrec2A.NGFR AAV6 donor or, as reference, with IL2RGrecPGK-GFP. IL2RGrec2A.NGFR AAV6 transduced, AAVS1-edited cells or untreated cells are also plotted as controls. (Top right) Number of total cells from left. (Bottom right) Percentage of IL2RG surface expression of HDR-edited cells as compared with WT/NHEJ-edited counterpart. B) Representative FACS plot showing ΔLNGFR+ cells within bulk CD34+ HSPC and the most primitive subpopulation (CD90+ cells) in presence or not of TALE#3-VPR-encoding mRNA electroporation (bottom and top, respectively). C) Percentage (Top) and relative fluorescence intensity (RFI) on edited only control (D+R) (Bottom) of GFP+ or ΔLNGFR+ cells at 24 and 36 hours, respectively, after gene targeting and transactivation procedure among HSPC subpopulations. D) (Left) Representative FACS plot of unsorted, sorted ΔLNGFR+ and ΔLNGFR− HSPCs 36 hours after targeting and transactivation procedure. (Top right) Experimental procedure for SMArTER strategy application in human CD34+ cells. (Middle right) Percentage of HDR-edited alleles and HSPC culture composition in unsorted, sorted ΔLNGFR+ and sorted ΔLNGFR− enriched populations. (Bottom right) Percentage of NGFR+ cells measured by FACS at 6 weeks post-transplant.

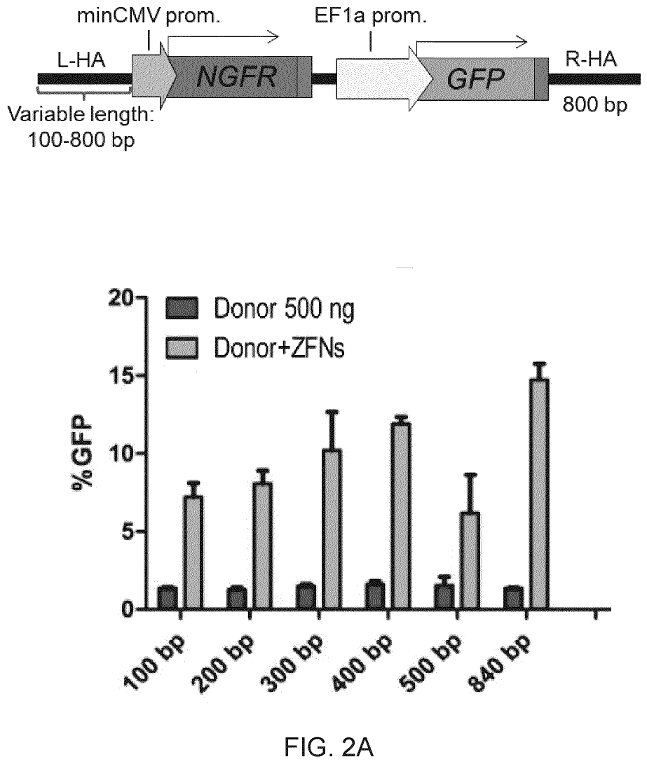

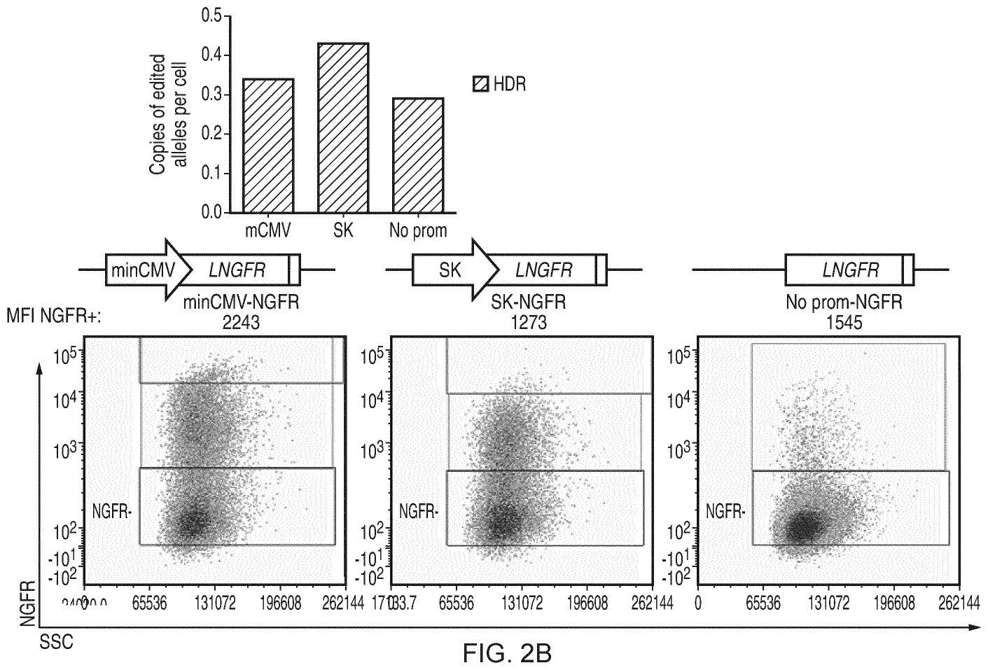

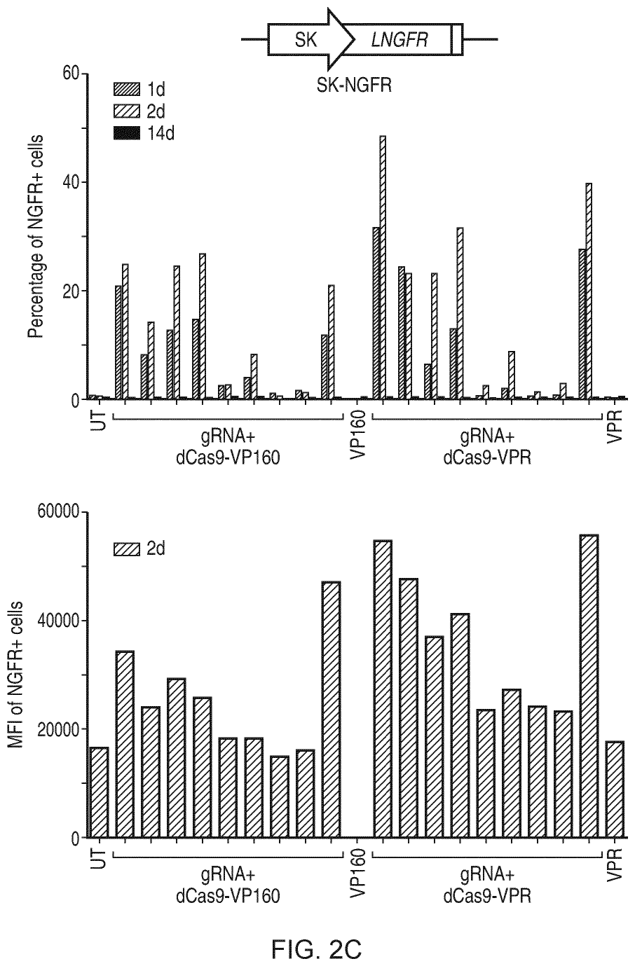

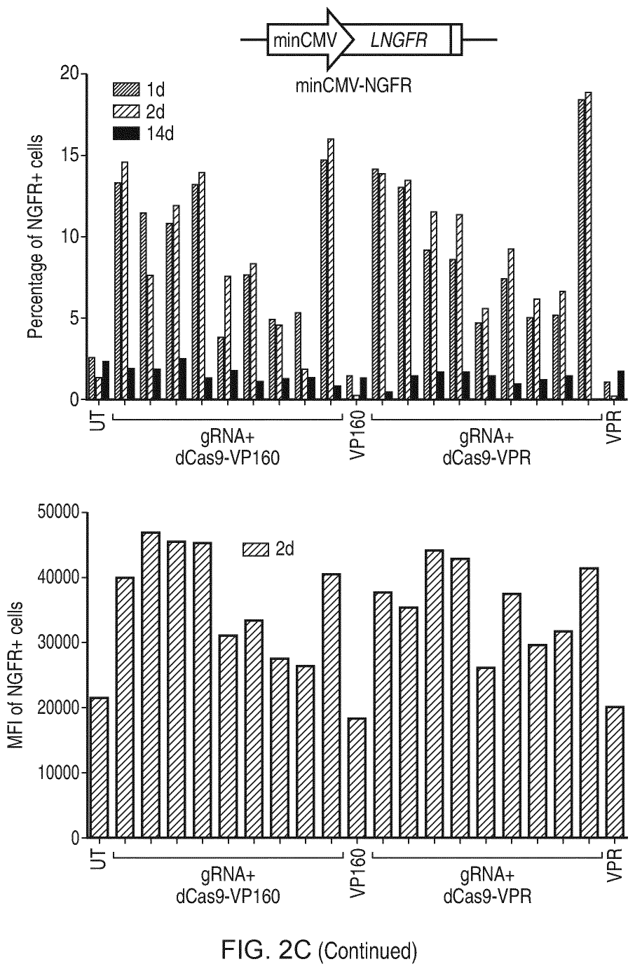

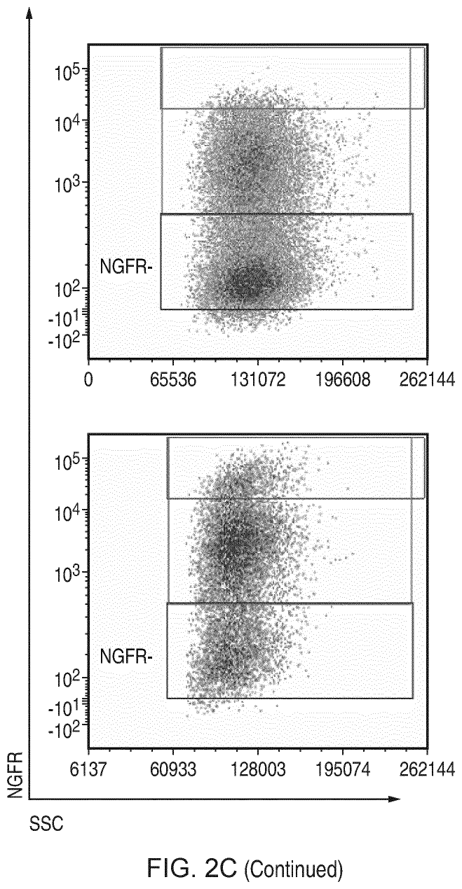

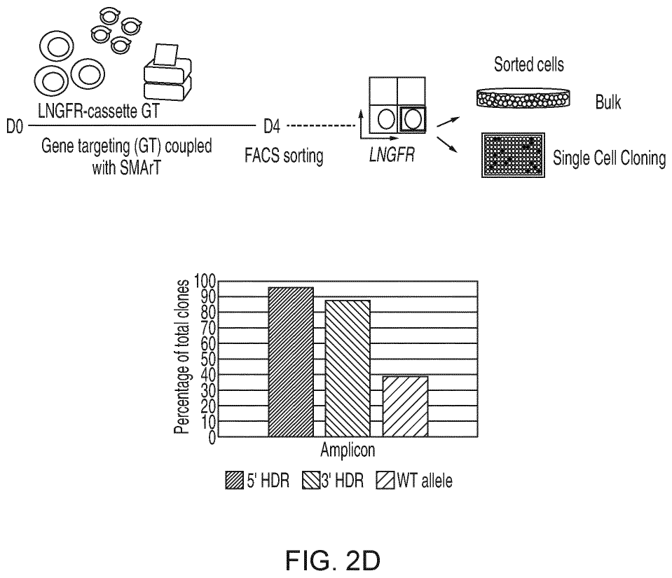

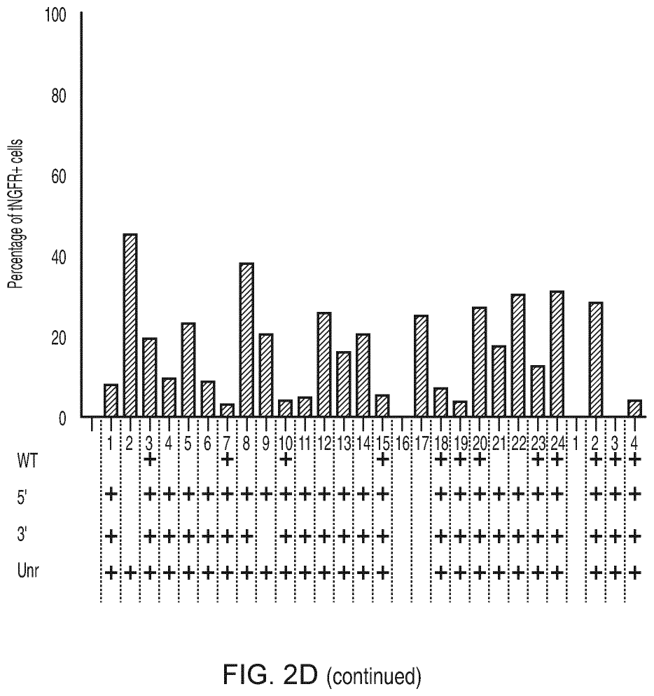

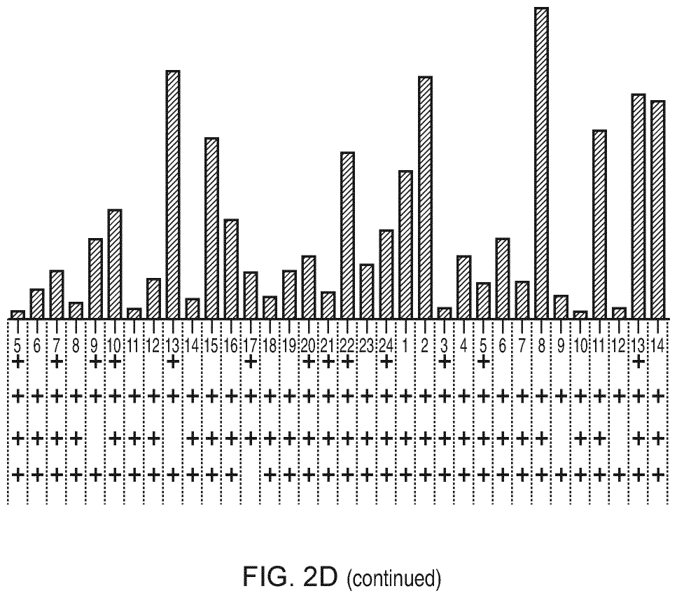

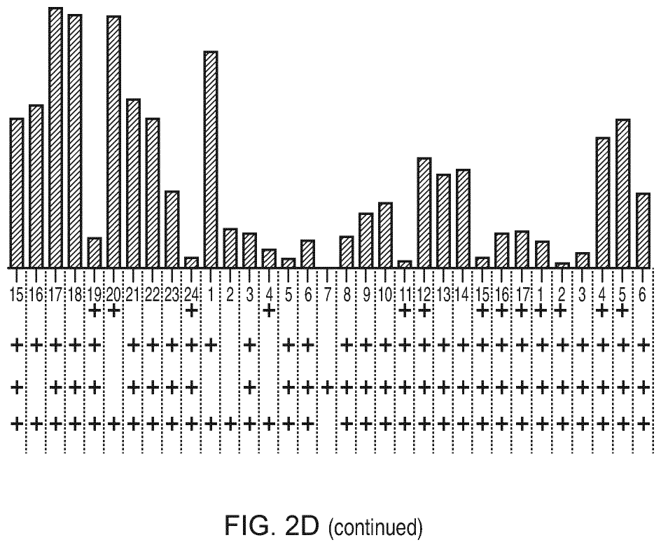

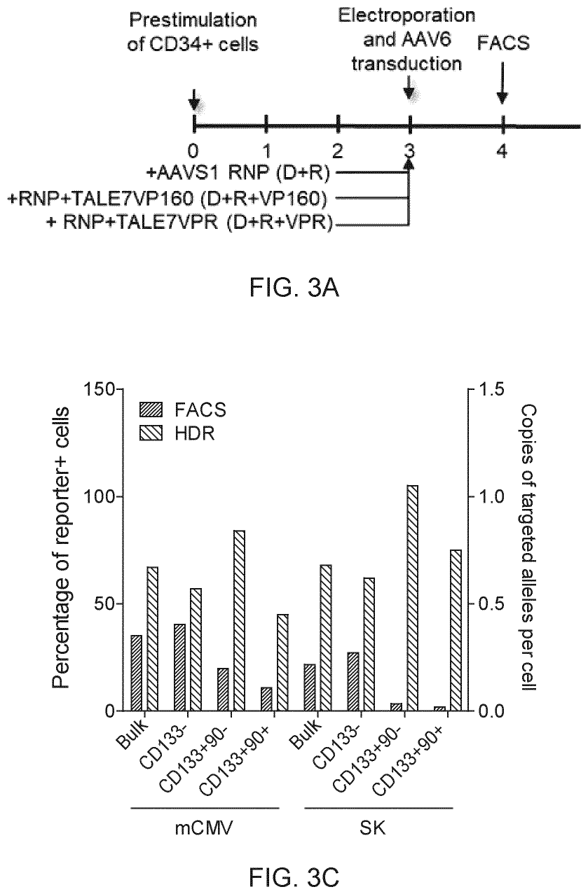

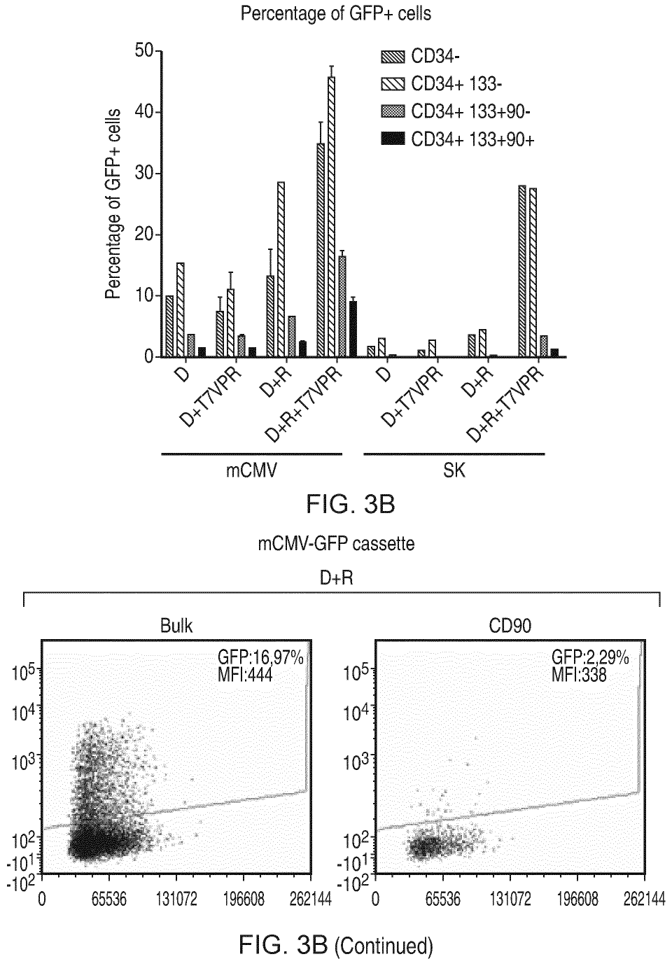

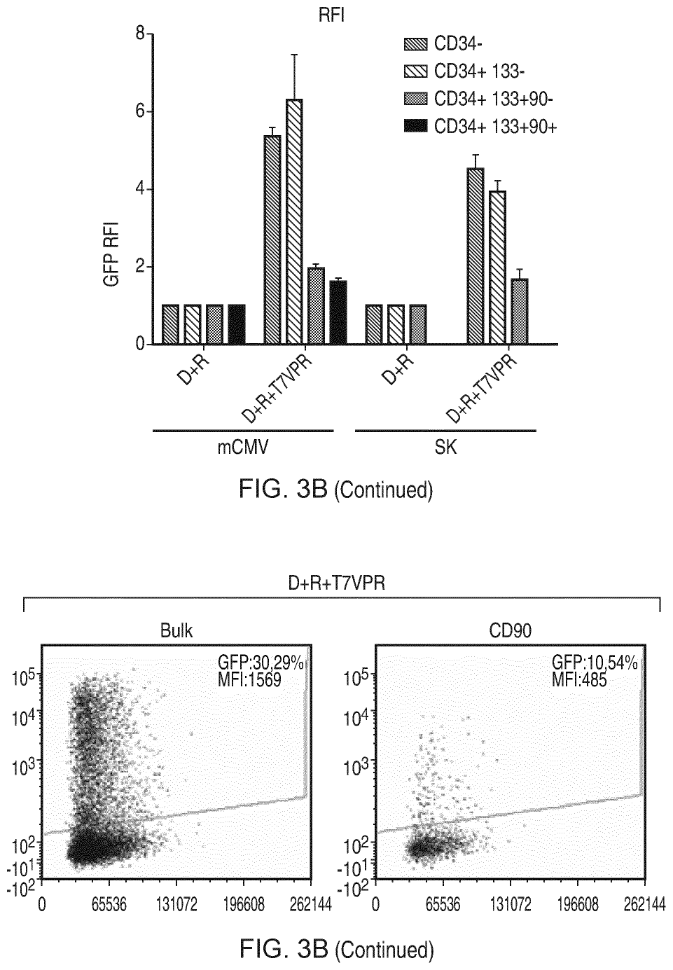

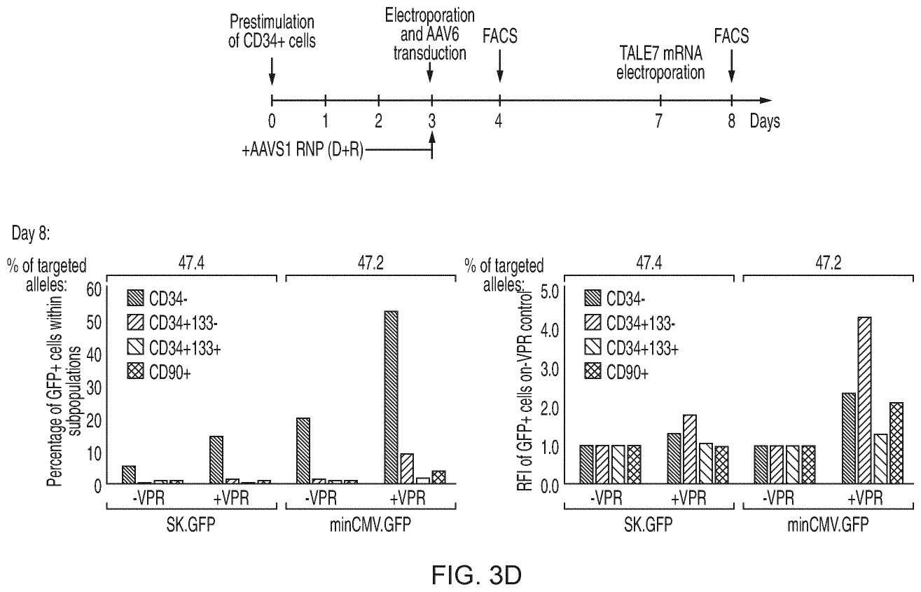

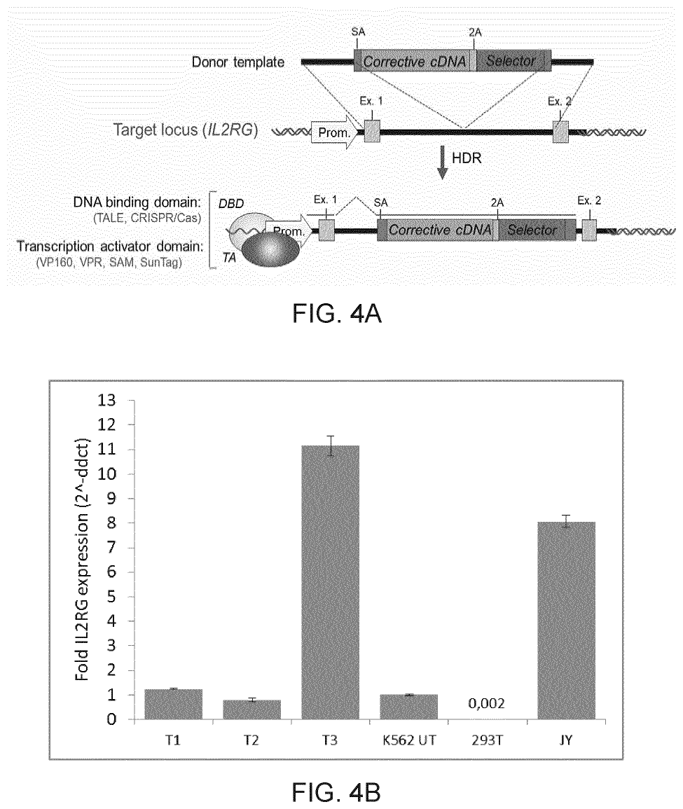

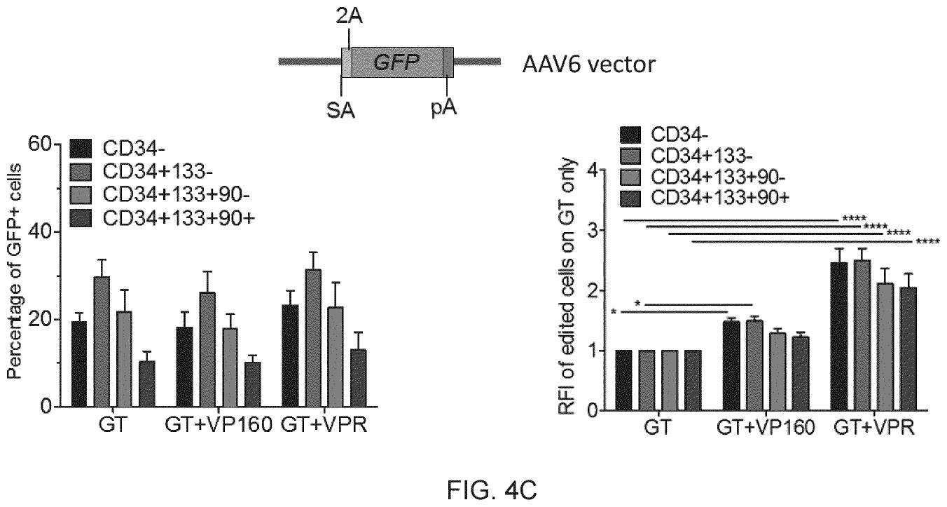

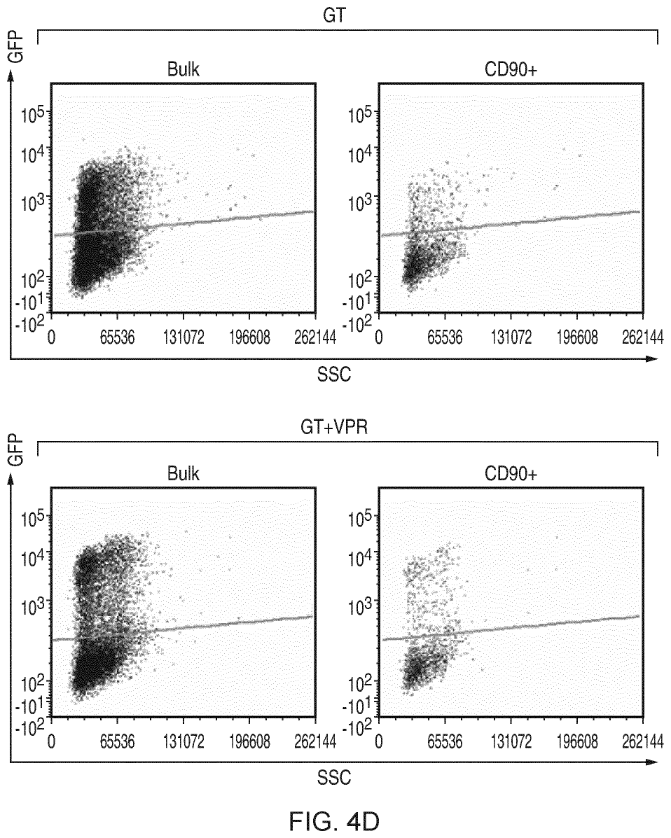

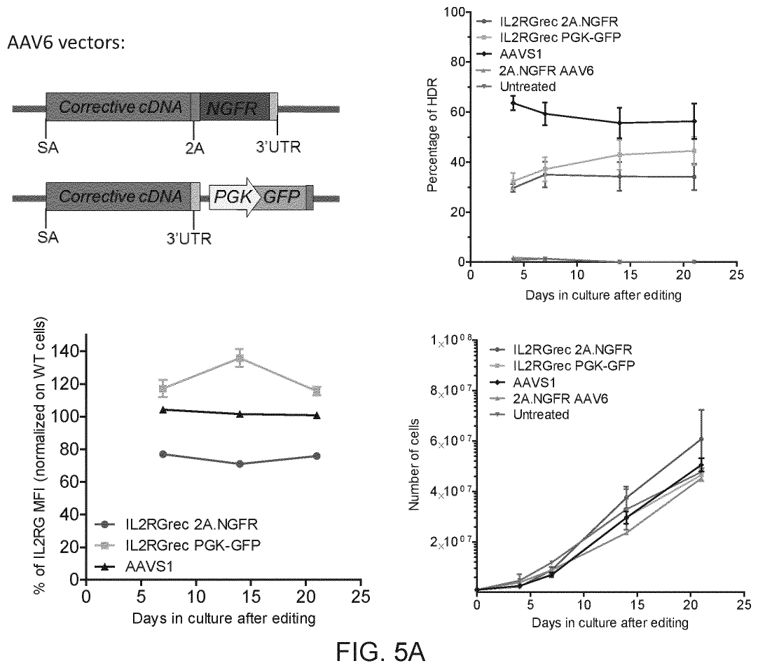

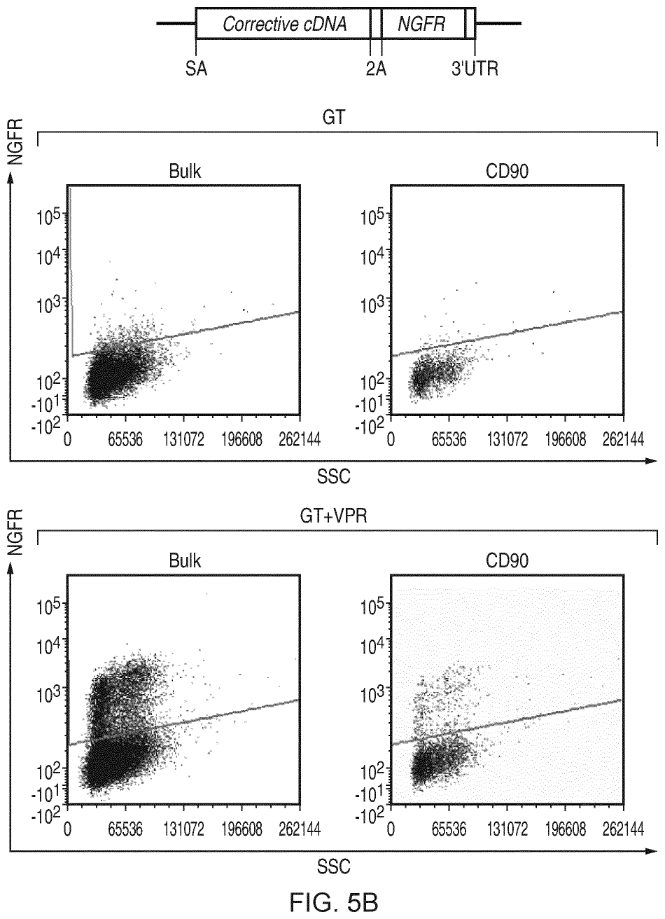

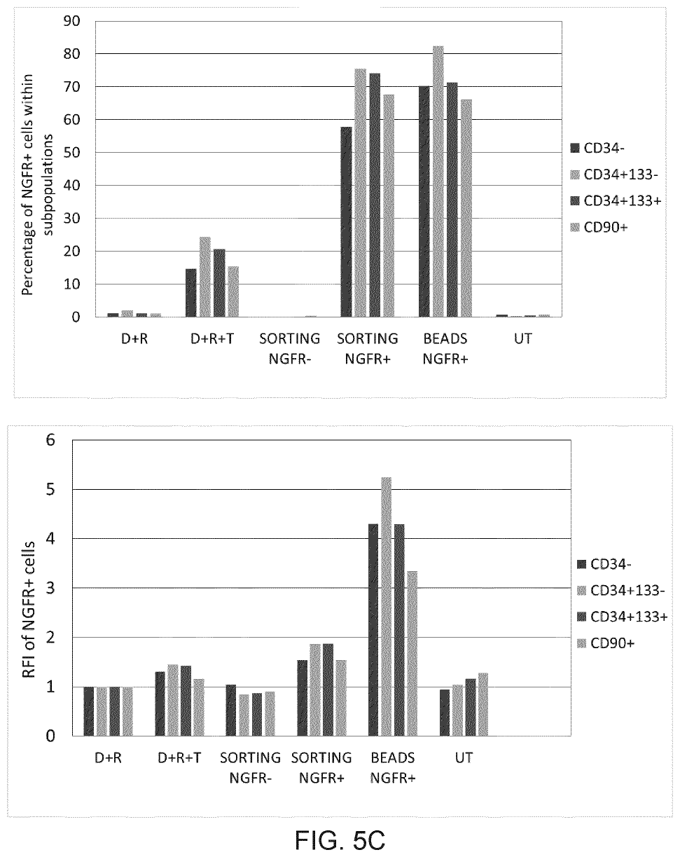

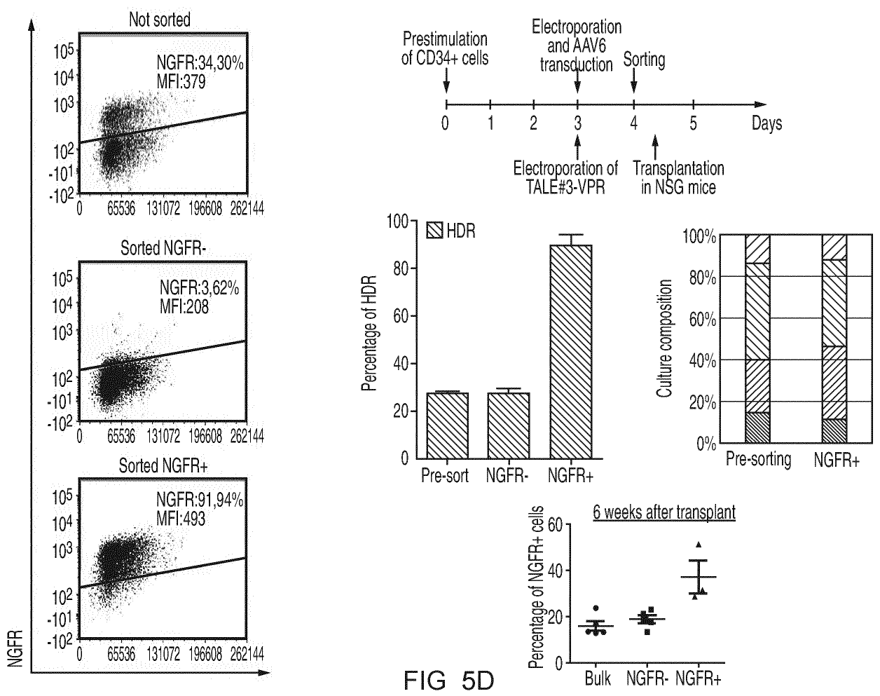

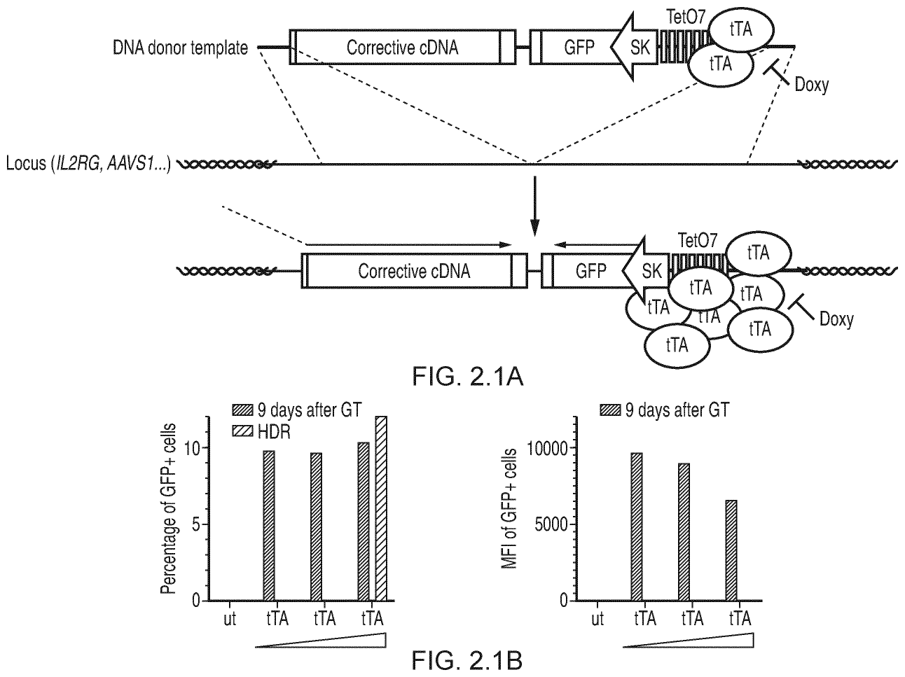

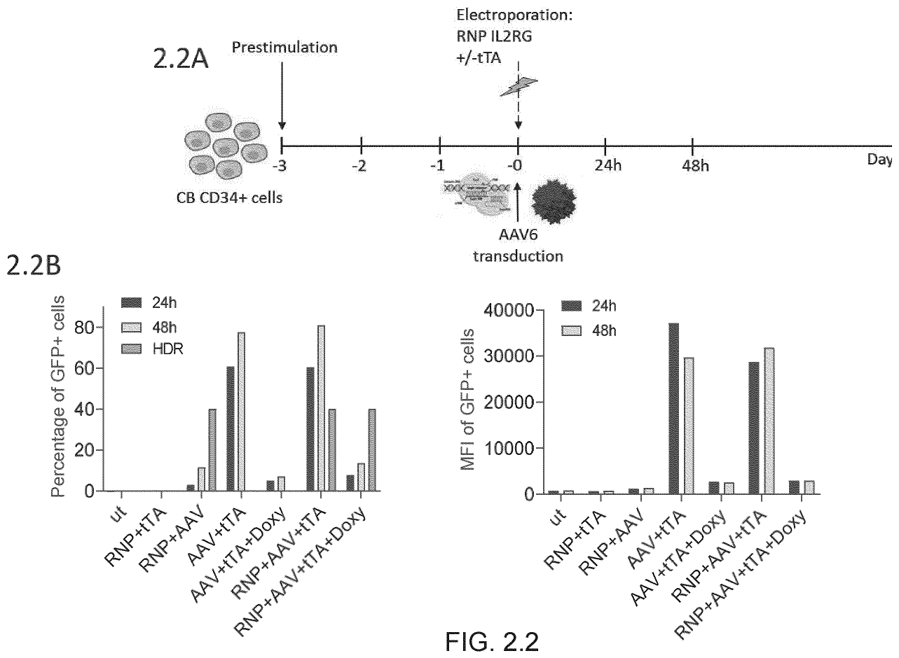

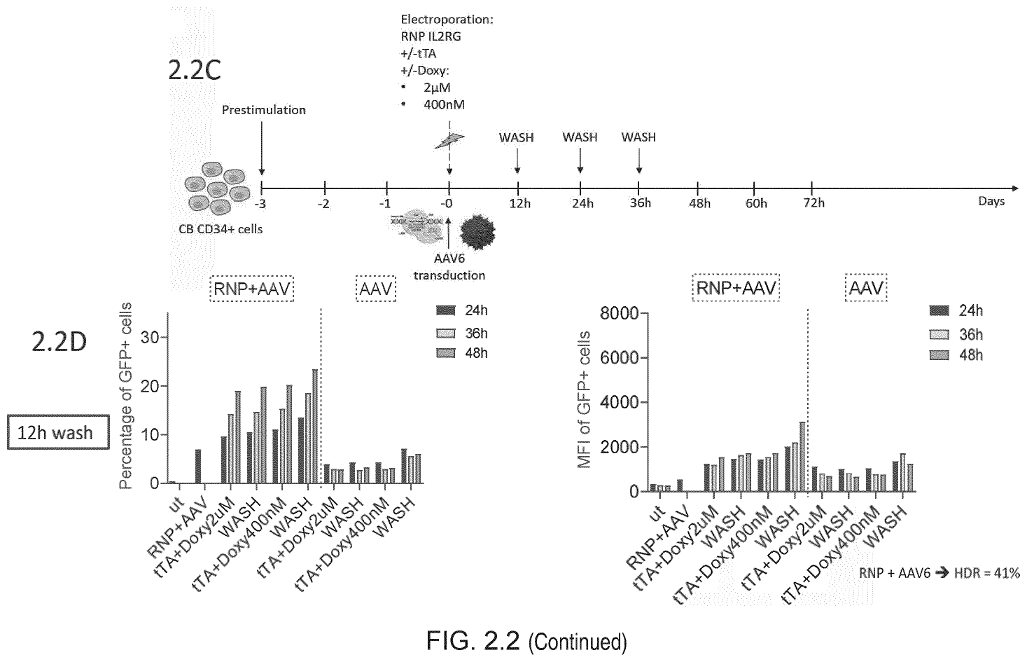

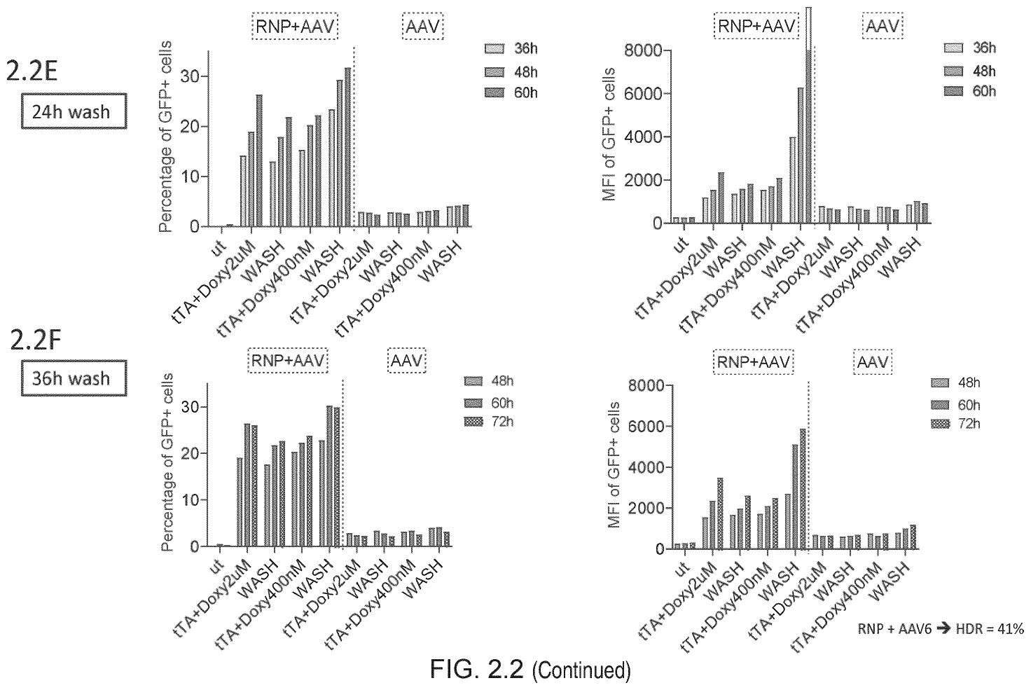

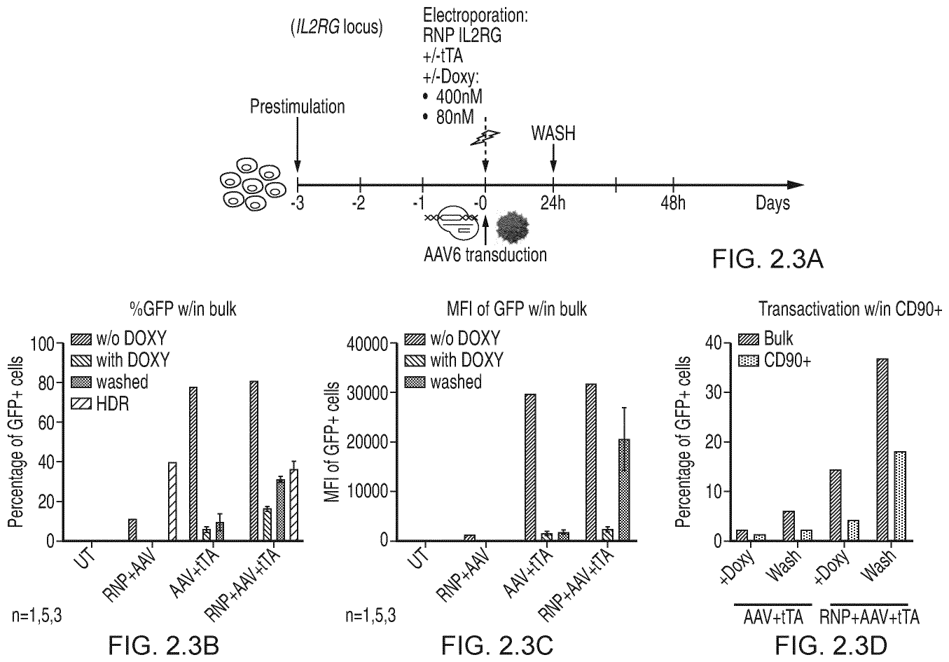

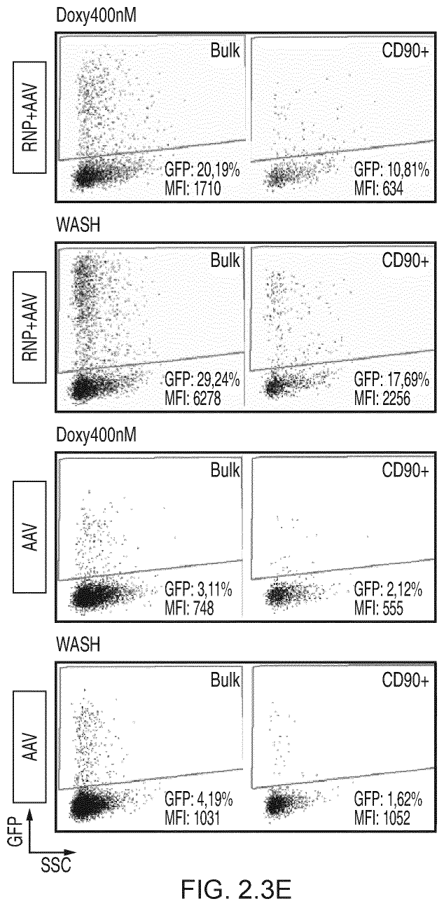

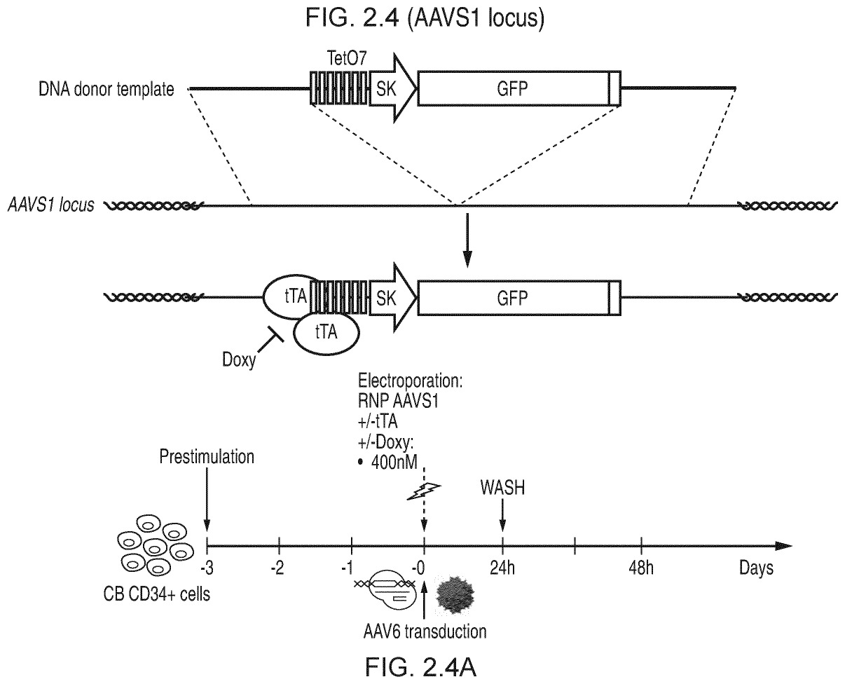

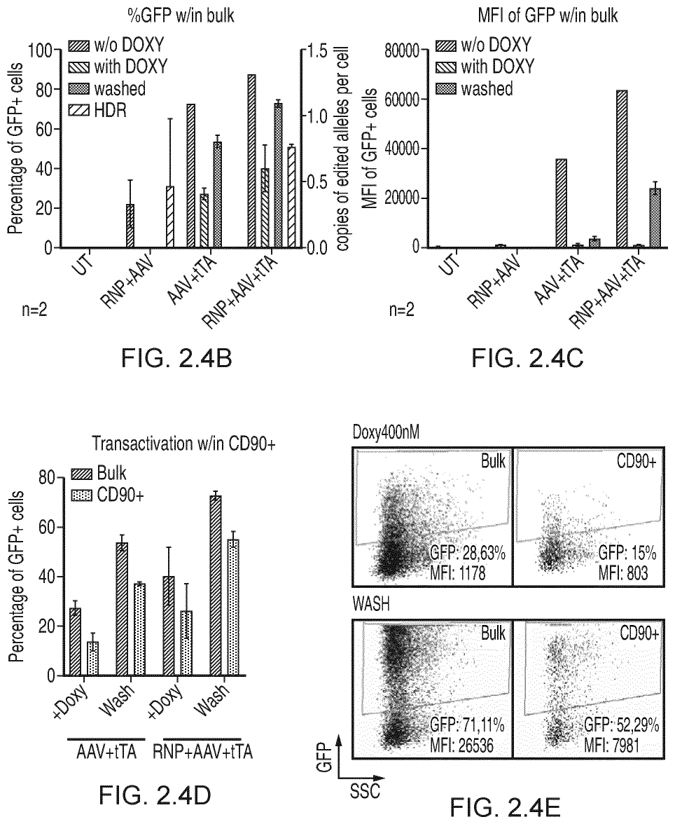

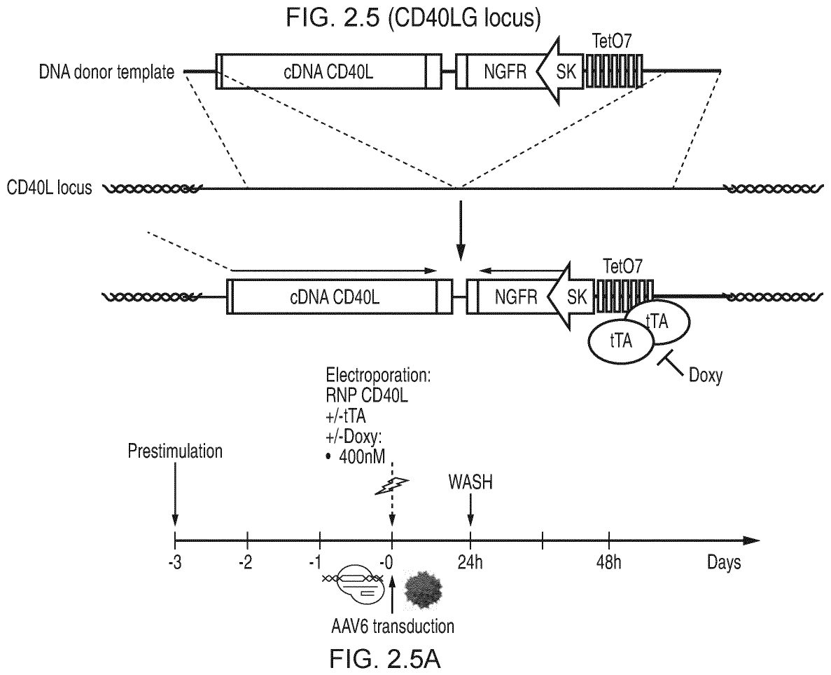

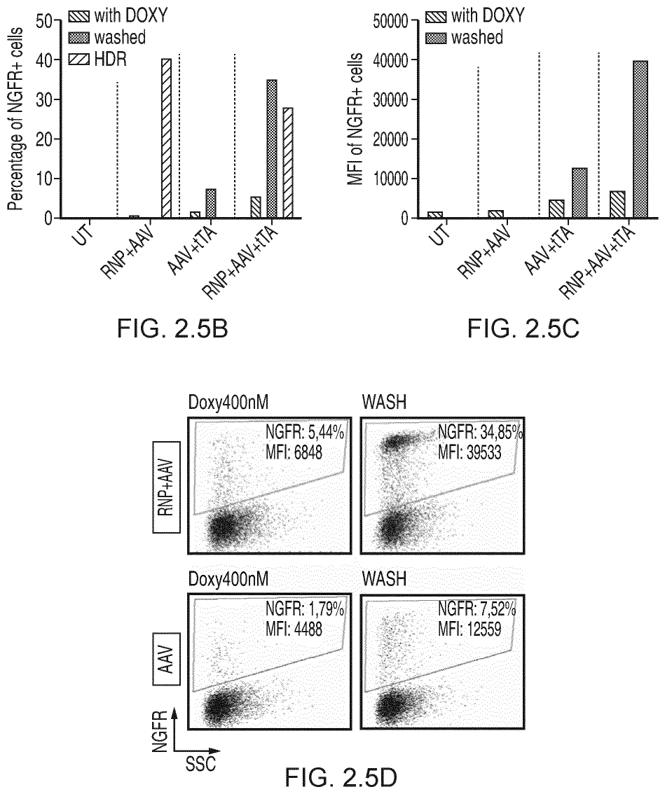

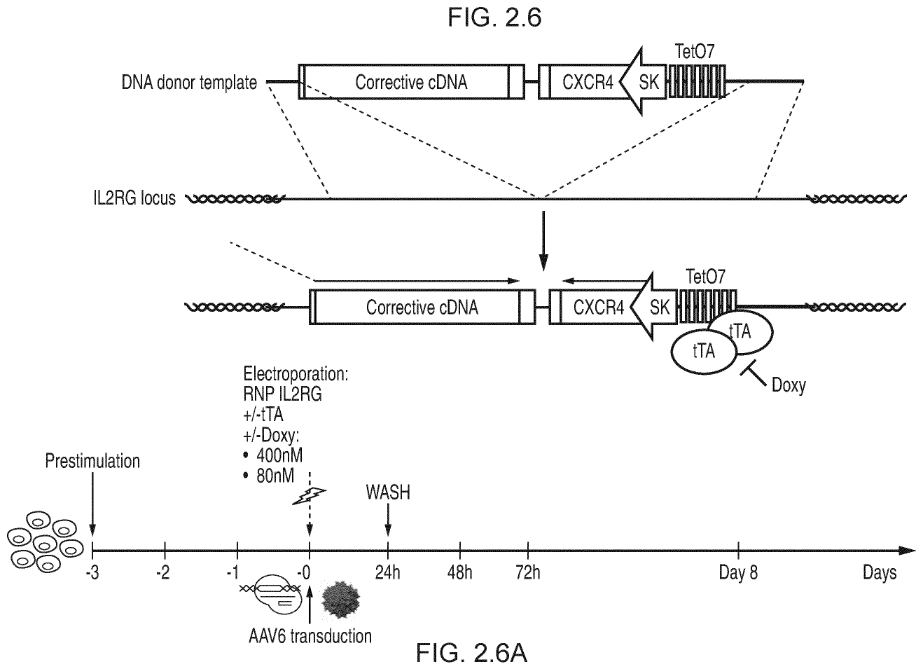

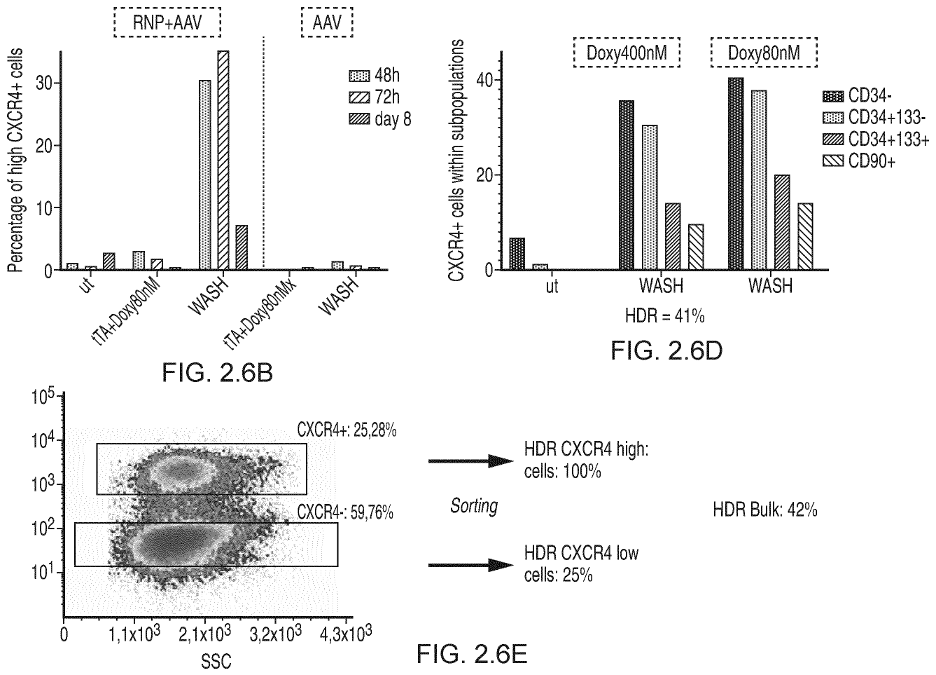

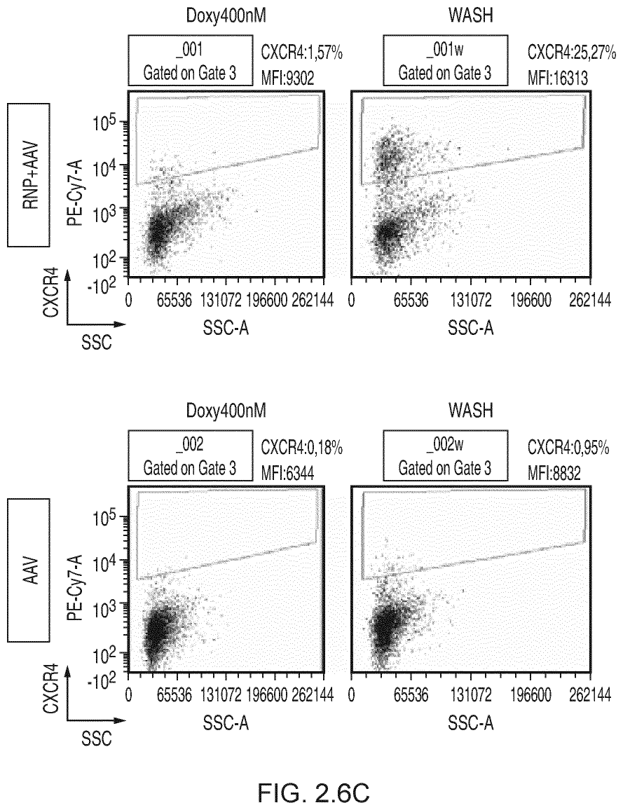

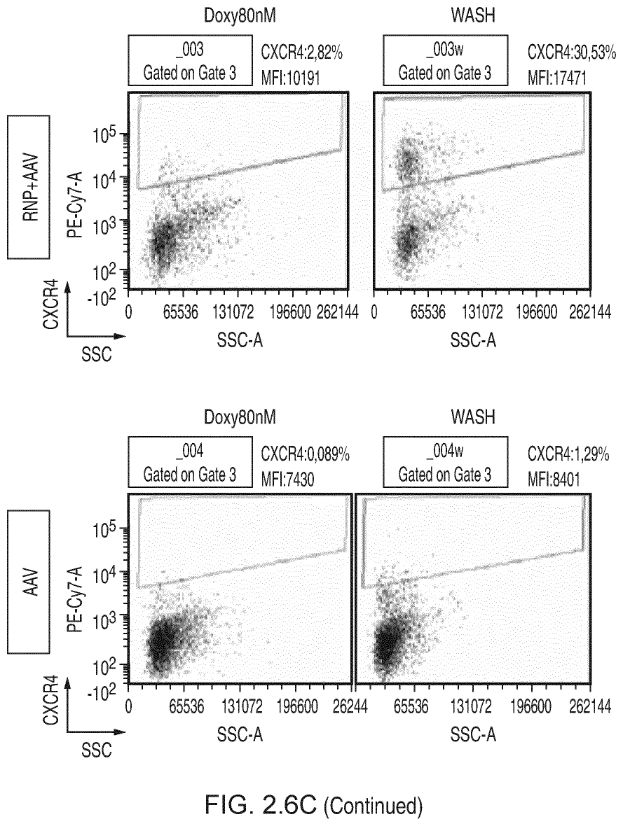

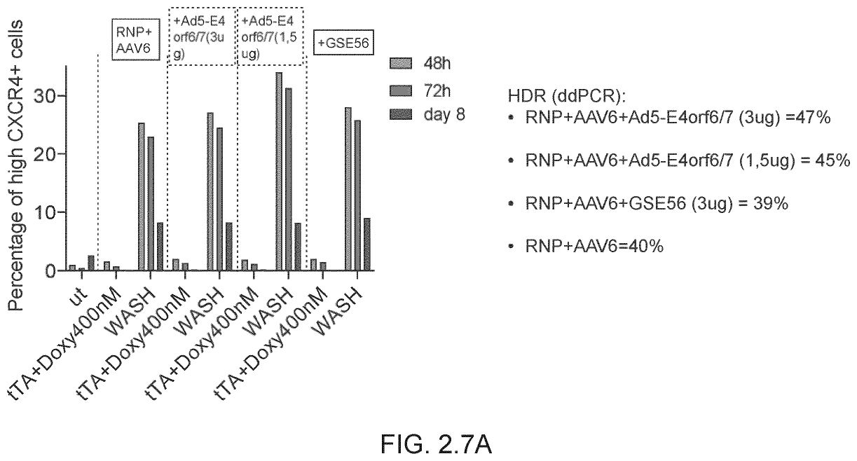

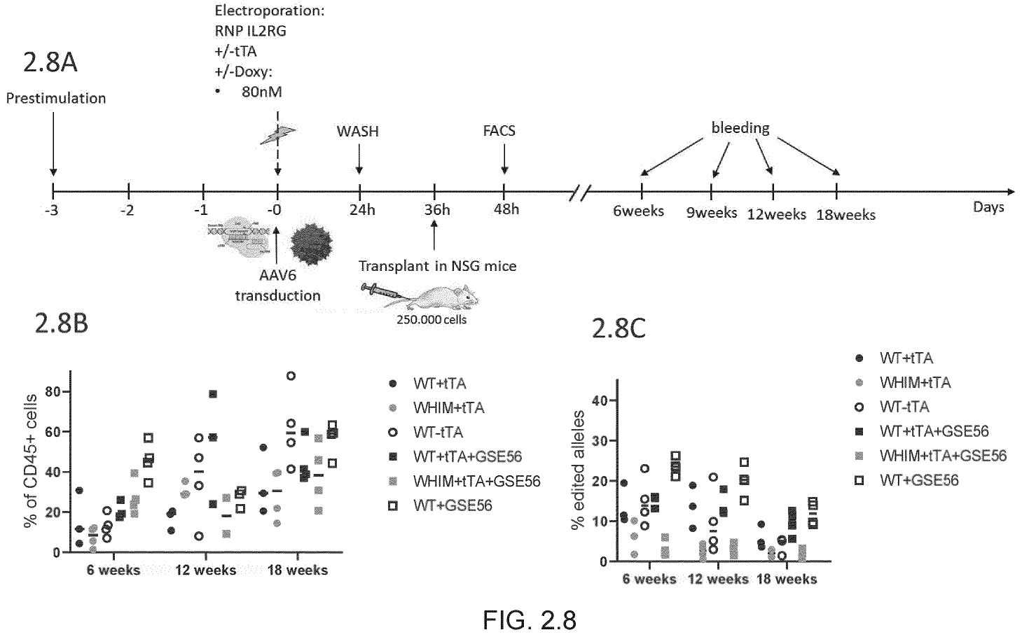

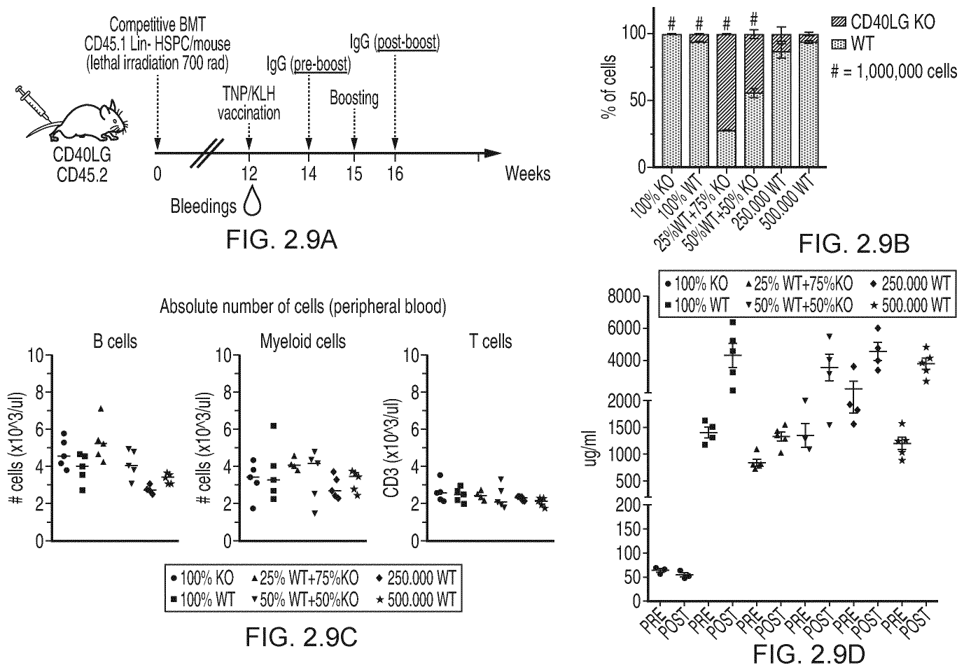

FIG. 2.1: Selection by Means of Artificial Transactivators with Doxycycline regulation (SMArT-D). A) Description of SMArT-D strategy. B) Percentage (left) and mean fluorescence intensity (MFI) (right) of GFP+ cells within K562 cells targeted with the SMArT-D construct at 24 h after electroporation of increasing amount of tTA (0.25 μg, 1 μg, 3 μg).

FIG. 2.2: Development of a protocol to transient transactivate IL2RG HDR-edited HSPCs. A) Experimental workflow in cord blood derived CD34+ cells: after three days of pre-stimulation cells were electroporated with/without IL2RG RNP+/−tTA, in presence or not of the AAV template for HDR and with the addiction or not of doxycycline (Doxy). B) Percentage (left) and MFI (right) of GFP+ cells within bulk population in untreated (UT), RNP+AAV (standard editing procedure), AAV+tTA, AAV+tTA+Doxy, RNP+AAV+tTA, RNP+AAV+tTA+Doxycycline measured at 24 and 48 hours after treatment. Percentage of HDR-edited cells measured at 4 days after treatment is indicated. C) Experimental design (as in FIG. 2.2A) for testing different doses of doxycycline (2 μM and 400 nM) and timing of doxycycline withdrawal. D) Percentage (left) and MFI (right) of GFP+ cells in “12-hours doxycycline withdrawal” conditions measured at 24 h, 36 h and 48 h after editing in the indicated conditions. Percentage of HDR-edited cells measured at 4 days after treatment is indicated. E) Percentage (left) and MFI (right) of GFP+ cells in “24-hours doxycycline withdrawal” conditions measured at 36 h, 48 h and 60 h after editing in the indicated conditions. Percentage of HDR-edited cells measured at 4 days after treatment is indicated. F) Percentage (left) and MFI (right) of GFP+ cells in “36-hours doxycycline withdrawal” conditions measured at 48 h, 60 h and 72 h after editing in the indicated conditions. Percentage of HDR-edited cells measured at 4 days after treatment is indicated.

FIG. 2.3: SMART-D strategy allows to enrich for IL2RG edited HSPCs. A) Optimized experimental workflow as described in FIG. 2.2C for IL2RG SMArT-D. B-C) Pool of 3 independent experiments showing the percentage (B) and MFI (C) of GFP+ cells in the indicated conditions with doxycycline withdrawal performed at 24 h after editing. Percentage of HDR-edited cells measured at 4 days after treatment is indicated. D) Percentage of GFP+ cells within the bulk population and the most primitive HSPC compartment (CD34+ CD133+ CD90+) in untargeted (AAV) and targeted (RNP+AAV) cells in presence of doxycycline or after doxycycline washout (n=3). E) Top: representative FACS plots showing the targeted bulk CD34+CD133+CD90+ population in presence of doxycycline and respective plots after doxycycline washout. Bottom: representative FACS plots showing the untargeted bulk and CD34+CD133+CD90+ population in presence of doxycycline and respective plots after doxycycline washout.

FIG. 2.4: SMART-D strategy allows to enrich for AAVS1 edited HSPCs. A) Left: SMArT-D strategy for the AAVS1 locus. Right: experimental workflow as described in FIG. 2.2C. B-C) Percentage of GFP+ cells (B) and MFI (C), as in FIG. 2.3B-C. D) Percentage of GFP+ cells within bulk and CD34+ CD133+ CD90+ cells, as in FIG. 2.3D (n=2). E) Representative FACS plots of targeted cells within bulk and CD34+ CD133+ CD90+ populations in presence of doxycycline or after washout.

FIG. 2.5: SMART-D strategy allows to enrich for CD40LG edited HSPCs. A) Left: SMArT-D strategy in CD40LG with truncated NGFR as selector gene. Right: experimental workflow as in FIG. 2.2C. B-C) Percentage (B) and MFI (C) of NGFR+ cells within untreated (UT), edited only (RNP+AAV), AAV+tTA and RNP+AAV+tTA each of them tested in presence of doxycycline or after washout. Percentage of HDR-edited cells measured at 4 days after treatment is indicated. D) Representative FACS plots showing transactivation in targeted and untargeted cells in presence or not of doxycycline.

FIG. 2.6: SMART-D strategy to in vivo select edited HSPCs. A) Left: SMArT-D strategy to target IL2RG locus with CXCR4 as biological selector. Right: experimental workflow as described in FIG. 2.2C with two different doxycycline doses. B) Percentage of CXCR4^{high} cells in RNP+AAV+tTA and AAV+tTA conditions in presence of doxycycline or after washout, measured at 24 h, 48 h and 8 days after gene editing procedure. C) Left: representative FACS plots within targeted and untargeted cells in presence of doxycycline 400 nM (left) and 80 nM (right) and after doxycycline washout. D) Percentage of high CXCR+ cells after doxycycline (400 nM or 80 nM) washout within indicated HSPCs subpopulations. E) FACS plot showing the gating strategy for CXCR4^{high} and CXCR4^{low} cells sorting, with the respective percentage of HDR-edited cells. Percentage of HDR in unsorted bulk population is also shown.

FIG. 2.7: Combination of SMArT-D strategy with Ad5-E4orf6/7 and/or GSE56 still allows to transactivate edited fraction. A) Percentage of CXCR5^{high} cells in indicated conditions measured in presence of doxycycline or after washout. Percentage of HDR-edited cells measured at 4 days after treatment is indicated.

FIG. 2.8: In vivo selection through SMArT-D strategy. A) Experimental design: gene editing is performed in presence or not of tTA after three days of pre-stimulation. Washout was performed at 24 h after editing. HSPC transplant in NSG mice was performed 12 h later (36 h after editing procedure). Bleedings were performed at 6, 12, 18 weeks after transplantation (n=3, 4, 4, 4, 4, 4). B) Percentage of human CD45+ cells at 6, 12 and 18 weeks from peripheral blood of transplanted mice in indicated conditions. C) Percentage of HDR-edited cells measured by digital droplet PCR in mice from FIG. 2.8B.

FIG. 2.9: Feasibility of a selection strategy in CD40LG mice. A) Experimental workflow and schematic representation of different groups (n=5, 5, 4, 5, 5, 5). B) Chimerism of wild-type (WT) and knock-out (KO) cells in peripheral blood of transplanted mice at 12 weeks. C) Absolute count of B cells, myeloid cells and T cells in peripheral blood at 12 weeks after transplant. D) Specific IgG response for TNP-KLH antigen measured by ELISA assay pre-boosting (after vaccination) and post-boosting.

### DETAILED DESCRIPTION

The terms “comprising”, “comprises” and “comprised of” as used herein are synonymous with “including” or “includes”; or “containing” or “contains”, and are inclusive or open-ended and do not exclude additional, non-recited members, elements or steps. The terms “comprising”, “comprises” and “comprised of” also include the term “consisting of”.

Genome Edited Cells

The term “genome edited cells” refers to a type of genetic engineering in which a nucleotide sequence is inserted, deleted, disrupted or replaced in the genome of a cell. The terms “genome edited cells”, “edited cells”, “gene edited cells”, “targeted gene therapy” and “gene editing” may be used interchangeably herein. Gene editing may be achieved using engineered nucleases (which herein may be referred to as the third component), which may be targeted to a desired site in a polynucleotide. Such nucleases may create site-specific double-strand breaks at desired locations, which may then be repaired through non-homologous end-joining (NHEJ) or homologous recombination (HR), resulting in targeted mutations. Such nucleases may be delivered to a target cell using viral vectors. Gene editing enlists the cell's own repair pathways to correct, disrupt, or add a target locus. For instance, in gene editing to correct a sequence, a template sequence for homology repair is provided and the cell's own repair pathways erase the mutation and replace it with the correct sequence. For instance, in gene editing to add a sequence, a template sequence for homology repair is provided and the cell's own repair pathways integrate a cassette allowing expression of a NOI in a site-specific manner. For instance, in gene editing to disrupt a gene a template sequence for homology repair is provided and the cell's own repair pathways mutate, insert or gene delete to disable the function of a gene.

Examples of suitable nucleases known in the art include zinc finger nucleases (ZFNs), transcription activator like effector nucleases (TALENs), the clustered regularly interspaced short palindromic repeats (CRISPR)/Cas system and the CRISPR/Cpf system (Gaj, T. et al. (2013) Trends Biotechnol. 31: 397-405; Sander, J. D. et al. (2014) Nat. Biotechnol. 32: 347-55).

Meganucleases (Silve, G. et al. (2011) Cur. Gene Ther. 11: 11-27) may also be employed as suitable nucleases for gene editing.

The CRISPR/Cas system is an RNA-guided DNA binding system (van der Oost et al. (2014) Nat. Rev. Microbiol. 12: 479-92), wherein the guide RNA (gRNA) may be selected to enable a Cas domain (such as Cas9) or a Cpf domain (such as Cpf 1) to be targeted to a specific sequence. Methods for the design of gRNAs are known in the art. Furthermore, fully orthogonal Cas9 proteins, as well as Cas9/gRNA ribonucleoprotein complexes and modifications of the gRNA structure/composition to bind different proteins, have been recently developed to simultaneously and directionally target different effector domains to desired genomic sites of the cells (Esvelt et al. (2013) Nat. Methods 10: 1116-21; Zetsche, B. et al. (2015) Cell pii: S0092-8674(15)01200-3; Dahlman, J. E. et al. (2015) Nat. Biotechnol. 2015 Oct. 5. doi: 10.1038/nbt.3390. [Epub ahead of print]; Zalatan, J. G. et al. (2015) Cell 160: 339-50; Paix, A. et al. (2015) Genetics 201: 47-54), and are suitable for use herein.

By “enrichment” of a population of cells for genome edited cells it is to be understood that the proportion (or concentration) of genome edited cells is increased within the population of cells. The concentration of other types of cells may be concomitantly reduced.

In some embodiments, the population of cells is an isolated population of cells. The term “isolated population” of cells as used herein may refer to the population of cells having been previously removed from the body. An isolated population of cells may be cultured and manipulated ex vivo or in vitro using standard techniques known in the art. An isolated population of cells may later be reintroduced into a subject. Said subject may be the same subject from which the cells were originally isolated or a different subject.

A population of cells may be enriched or purified selectively for cells that exhibit a specific phenotype or characteristic, and from other cells which do not exhibit that phenotype or characteristic, or exhibit it to a lesser degree. For example, a population of cells that expresses a specific selector (such as ΔLNGFR, NGFR, truncated NGFR, MGMT, CD19, EGFR, c-Kit, CXCR4, CXCR4 WHIM, CD47 and eGFP) may be purified from a starting population of cells. Alternatively, or in addition, a population of cells that does not express another selector may be purified.

Enrichment or purification may result in the population of cells being substantially pure of other types of cell.

Enriching or purifying for cells or a population of cells expressing a specific selector (e.g. ΔLNGFR, NGFR, truncated NGFR, MGMT, CD19, EGFR, c-Kit, CXCR4, CXCR4 WHIM, CD47 and eGFP) may be achieved by using an agent that binds to that marker, preferably substantially specifically to that marker. For example, a population of genome edited cells could be marked by antibodies, proteins or aptamers that are specific for the marker and that are conjugated, directly or indirectly, with a fluorescent dye or paramagnetic beads. Thus, selection of the marked cells could be performed by fluorescence-activated cell sorting, magnetic columns, affinity tag purification or microscopy-based techniques.

In some embodiments, the nucleotide sequence encodes a selector selected from the group consisting of ΔLNGFR, NGFR, MGMT, CD19, EGFR, c-Kit, CXCR4, CXCR4 WHIM, CD47 and eGFP.

Transient Expression or Transient Upregulation

When the second component is an ETT polypeptide, the ETT polypeptide is transiently present in the cell or population of cells. Without wishing to be bound by theory, the transient presence of the ETT polypeptide occurs due to intracellular degradation of this second component.

When the second component is a nucleotide sequence encoding the ETT polypeptide, the nucleotide sequence is transiently expressed in the cell or population of cells. Without wishing to be bound by theory, the transient expression of the nucleotide sequence encoding the ETT polypeptide occurs due to intracellular degradation of this second component. In addition, the transient expression of the nucleotide sequence encoding the ETT polypeptide results in the short term production of the ETT polypeptide. In turn, the ETT polypeptide is only transiently present in the cell or population of cells as the ETT polypeptide undergoes intracellular degradation.

Binding sites for the ETT polypeptide (ETT binding sites) are operably linked to the target locus in the genome of the cell. In some embodiments, the at least one ETT binding site is upstream of the target locus. In other embodiments the at least one ETT binding sites are downstream of the target locus. The presence of the ETT binding site enables the expression of the nucleotide sequence encoding the selector inserted at the target locus when the ETT polypeptide is present in the cell. Without wishing to be bound by theory, in genome edited cells, ETT bound to the ETT binding site can act on an endogenous promoter operably linked to the target locus or an exogenous promoter (such as a minimal promoter) operably linked to the nucleotide sequence encoding the selector inserted into the target locus. Expression of the nucleotide sequence encoding the selector can be modulated independently from expression of the NOI in genome edited cells unless the nucleotide sequence encoding the selector and the NOI are the same sequence.

The transient presence of the ETT polypeptide in a cell or population of cells enables the transient expression or the transient upregulation of the inserted nucleotide sequence encoding the selector when the nucleotide sequence encoding the selector is inserted into the target locus (this may be referred to as “on-site” insertion). If the nucleotide sequence encoding the selector is inserted into a non-target locus (this may be referred to as “off-site” insertion) then the nucleotide sequence encoding the selector is not expressed.

The transient expression of the nucleotide sequence encoding the ETT polypeptide enables the transient expression or the transient upregulation of the inserted nucleotide sequence encoding the selector when the nucleotide sequence encoding the selector is inserted into the target locus (this may be referred to as “on-site” insertion). If the nucleotide sequence encoding the selector is inserted into a non-target locus (this may be referred to as “off-site” insertion) then the nucleotide sequence encoding the selector is not expressed.

In some embodiments, the transient expression or the transient upregulation of the inserted nucleotide sequence encoding the selector requires the presence of a modulator in the cell. In other embodiments, the transient expression or the transient upregulation of the inserted nucleotide sequence encoding the selector does not require the presence of a modulator in the cell.

The term “transiently express a nucleotide sequence encoding a selector” as used herein refers to a temporary expression of the nucleotide sequence encoding the selector. The transient expression of the selector enables the selection of the genome edited cells during this period of selector expression.

The term “transiently upregulate a nucleotide sequence encoding a selector” as used herein refers to a temporary increase in the expression of the nucleotide sequence encoding the selector. The transient upregulation of the selector enables the selection of the genome edited cells during this period of selector upregulated expression.

The upregulation of a selector refers to an increase in the level of expression of the selector within the population of cells that contain targeted integration of the NOI in comparison to the population of cells that does not contain integration of NOI in the target site under otherwise identical conditions.

Advantageously, the genome editing method of the present invention results in the expression or the upregulation of the selector for a short period of time. During this period of time, the genome edited cells can be selected.

For gene therapy, for instance, advantageously the genome edited cells do not express the selector or do not have upregulated expression of the selector when the cells are introduced or transplanted into the subject.

In some embodiments, the ETT polypeptide or the nucleotide sequence encoding the ETT polypeptide is transiently present in the cell or population of cells for about 6 hours to about 7 days, about 6 hours to about 5 days, about 6 hours to about 4 days, about 6 hours to about 3 days, about 6 hours to about 48 hours, about 6 hours to about 36 hours, about 6 hours to about 24 hours, or about 6 hours to about 12 hours.

In some embodiments, the ETT polypeptide or the nucleotide sequence encoding the ETT polypeptide is transiently expressed in the cell or population of cells for about 6 hours to about 7 days, about 6 hours to about 5 days, about 6 hours to about 4 days, about 6 hours to about 3 days, about 6 hours to about 48 hours, about 6 hours to about 36 hours, about 6 hours to about 24 hours, or about 6 hours to about 12 hours.

Selection of Cells

The method according to the present invention comprises selecting the genome edited cells which transiently express or transiently upregulate a nucleotide sequence encoding a selector.

A number of techniques for selecting a cell or a population of cells expressing a selector are known in the art. These include magnetic bead-based separation technologies (e.g. closed-circuit magnetic bead-based separation), flow cytometry, fluorescence-activated cell sorting (FACS), affinity tag purification (e.g. using affinity columns or beads, such biotin columns to separate avidin-labelled agents) and microscopy-based techniques.

It may also be possible to perform the selection using a combination of different techniques, such as a magnetic bead-based separation step followed by sorting of the resulting population of cells for one or more additional (positive or negative) markers by flow cytometry.

Clinical grade separation may be performed, for example, using the CliniMACS® system (Miltenyi) or CliniMACS® Prodigy system (Miltenyi). These are two examples of a closed-circuit magnetic bead-based separation technology.

In the present invention, cells or populations of cells which do not transiently express or transiently upregulate a nucleotide sequence encoding a selector will not be selected.

The technique employed for selecting genome edited cells is preferably one which is amenable to automation and/or high throughput screening.

In some embodiments, the genome edited cells are selected by flow cytometry (such as Fluorescence-activated cell sorting) or magnetic bead separation.

In some embodiments, the genome edited cells are selected by magnetic bead-based separation technologies.

In some embodiments, the genome edited cells are selected by closed-circuit magnetic bead-based separation.

Table B details examples of antibodies suitable for use in selecting for genome edited cells by, for instance, flow cytometry.

Cell Types

In some embodiments, the cell is a mammalian cell. Preferably the cell is a human cell.

In some embodiments, the population of cells are mammalian cells. Preferably the population of cells are human cells.

In some embodiments, the cell is a genome edited cell.

In some embodiments, the population of cells are the starting population of cells. The starting population of cells undergo genome editing according to the method of the present invention. In other embodiments, the population of cells are a population of genome edited cells.

In some embodiments, the cell or population of cells is a hematopoietic stem cell (HSC), a hematopoietic progenitor cell (HPC), a myeloid/monocyte-committed progenitor cell, a macrophage or monocyte, a T or B cell lymphocyte, an embryonic stem cell (ESC), induced pluripotent stem cell (iPSC), an epidermal stem cell, a limbal stem cell culture, a mesenchymal stromal cell (MSC), a neural stem cell (NSC), or a mesoangioblast.

In some embodiments, the population of cells are hematopoietic stem cells (HSCs), hematopoietic progenitor cells (HPCs), myeloid/monocyte-committed progenitor cells, macrophages or monocytes, T or B cell lymphocytes, embryonic stem cells (ESCs), induced pluripotent stem cells (iPSCs), epidermal stem cells, limbal stem cell culture, mesenchymal stromal cells (MSCs), neural stem cells (NSCs), mesoangioblasts or a mixture thereof.

A stem cell is able to differentiate into many cell types. A cell that is able to differentiate into all cell types is known as totipotent. In mammals, only the zygote and early embryonic cells are totipotent. Stem cells are found in most, if not all, multicellular organisms. They are characterised by the ability to renew themselves through mitotic cell division and differentiate into a diverse range of specialised cell types. The two broad types of mammalian stem cells are embryonic stem cells that are isolated from the inner cell mass of blastocysts, and adult stem cells that are found in adult tissues. In a developing embryo, stem cells can differentiate into all of the specialised embryonic tissues. In adult organisms, stem cells and progenitor cells act as a repair system for the body, replenishing specialised cells, but also maintaining the normal turnover of regenerative organs, such as blood, skin or intestinal tissues.

Haematopoietic stem cells (HSCs) are multipotent stem cells that may be found, for example, in peripheral blood, bone marrow and umbilical cord blood. HSCs are capable of self-renewal and differentiation into any blood cell lineage. They are capable of recolonising the entire immune system, and the erythroid and myeloid lineages in all the haematopoietic tissues (such as bone marrow, spleen and thymus). They provide for life-long production of all lineages of haematopoietic cells.

Haematopoietic progenitor cells have the capacity to differentiate into a specific type of cell. In contrast to stem cells however, they are already far more specific: they are pushed to differentiate into their “target” cell. A difference between stem cells and progenitor cells is that stem cells can replicate indefinitely, whereas progenitor cells can only divide a limited number of times. Haematopoietic progenitor cells can be rigorously distinguished from HSCs only by functional in vivo assay (i.e. transplantation and demonstration of whether they can give rise to all blood lineages over prolonged time periods).

Haematopoietic Stem and Progenitor Cell (HSPC) Sources

A population of haematopoietic stem and/or progenitor cells may be obtained from a tissue sample.

For example, a population of haematopoietic stem and/or progenitor cells may be obtained from peripheral blood (e.g. adult and foetal peripheral blood), umbilical cord blood, bone marrow, liver or spleen. Preferably, these cells are obtained from peripheral blood or bone marrow. They may be obtained after mobilisation of the cells in vivo by means of growth factor treatment.

Mobilisation may be carried out using, for example, G-CSF, plerixaphor or combinations thereof. Other agents, such as NSAIDs and dipeptidyl peptidase inhibitors, may also be useful as mobilising agents.

With the availability of the stem cell growth factors GM-CSF and G-CSF, most haematopoietic stem cell transplantation procedures are now performed using stem cells collected from the peripheral blood, rather than from the bone marrow. Collecting peripheral blood stem cells provides a bigger graft, does not require that the donor be subjected to general anaesthesia to collect the graft, results in a shorter time to engraftment and may provide for a lower long-term relapse rate.

Bone marrow may be collected by standard aspiration methods (either steady-state or after mobilisation), or by using next-generation harvesting tools (e.g. Marrow Miner).

In addition, haematopoietic stem and progenitor cells may also be derived from induced pluripotent stem cells.

HSC Characteristics

HSCs are typically of low forward scatter and side scatter profile by flow cytometric procedures. Some are metabolically quiescent, as demonstrated by Rhodamine labelling which allows determination of mitochondrial activity. HSCs may comprise certain cell surface markers such as CD34, CD45, CD133, CD90, CD201 and CD49f. They may also be defined as cells lacking the expression of the CD38 and CD45RA cell surface markers. However, expression of some of these markers is dependent upon the developmental stage and tissue-specific context of the HSC. Some HSCs called “side population cells” exclude the Hoechst 33342 dye as detected by flow cytometry. Thus, HSCs have descriptive characteristics that allow for their identification and isolation.

Negative Markers

CD38 is the most established and useful single negative marker for human HSCs.

Human HSCs may also be negative for lineage markers such as CD2, CD3, CD14, CD16, CD19, CD20, CD24, CD36, CD56, CD66b, CD271 and CD45RA. However, these markers may need to be used in combination for HSC enrichment.

By “negative marker” it is to be understood that human HSCs lack the expression of these markers.

Positive Markers

CD34 and CD133 are the most useful positive markers for HSCs.

Some HSCs are also positive for lineage markers such as CD90, CD49f and CD93. However, these markers may need to be used in combination for HSC enrichment.

By “positive marker” it is to be understood that human HSCs express these markers.

In some embodiments, the haematopoietic stem and progenitor cells are CD34+CD38− cells.

Differentiated Cells

A differentiated cell is a cell which has become more specialised in comparison to a stem cell or progenitor cell. Differentiation occurs during the development of a multicellular organism as the organism changes from a single zygote to a complex system of tissues and cell types. Differentiation is also a common process in adults: adult stem cells divide and create fully-differentiated daughter cells during tissue repair and normal cell turnover. Differentiation dramatically changes a cell's size, shape, membrane potential, metabolic activity and responsiveness to signals. These changes are largely due to highly-controlled modifications in gene expression. In other words, a differentiated cell is a cell which has specific structures and performs certain functions due to a developmental process which involves the activation and deactivation of specific genes. Here, a differentiated cell includes differentiated cells of the haematopoietic lineage such as monocytes, macrophages, neutrophils, basophils, eosinophils, erythrocytes, megakaryocytes/platelets, dendritic cells, T cells, B-cells and NK-cells. For example, differentiated cells of the haematopoietic lineage can be distinguished from stem cells and progenitor cells by detection of cell surface molecules which are not expressed or are expressed to a lesser degree on undifferentiated cells. Examples of suitable human lineage markers include CD33, CD13, CD14, CD15 (myeloid), CD19, CD20, CD22, CD79a (B), CD36, CD71, CD235a (erythroid), CD2, CD3, CD4, CD8 (T) and CD56 (NK).

The cell or population of cells for use herein may be cultured in any medium suitable for maintaining and/or growing the cells.

Provasi et al (Nat Med 2012 18(5) 807-815; PMID: 22466705) disclose media which are suitable for maintaining and/or growing T cells.

In some embodiments, the cell or population of cells is a K562 cell.

In other embodiments, the cell or population of cells is a CD34+ cell.

Introduction of Components in Cells

The method according to the present invention comprises introducing into a cell or a population of cells at least one first component, at least one second component and at least one third component. The first component, the second component and the third component are discrete and distinct components. Accordingly, at least 3 components are introduced into a cell or a population of cells by the method according to the present invention. In some embodiments, the components used in the method of the present invention are not limited to these three components; additional components might be used in the method depending on, for instance, the specific gene or genes to be targeted.

As used herein, the term “introducing” refers to methods for inserting the components (e.g. foreign DNA or RNA or polypeptide) into a cell. As used herein the term “introducing” includes both transduction and transfection methods. Transfection is the process of introducing the components (e.g. nucleic acids) into a cell by non-viral methods. Transduction is the process of introducing foreign DNA or RNA into a cell via a viral vector.

In some embodiments, the first component and/or second component and/or third component and/or additional component is introduced to the cell by electroporation. In some embodiments, the first component and/or second component and/or third component and/or additional component are introduced to the cell by chemical-based transfection (such as calcium phosphate, cationic polymers (PEI) and liposomes). In some embodiments, the first component and/or second component and/or third component and/or additional component is introduced to the cell by transduction. Suitably, the first component and/or second component and/or third component and/or additional component may be introduced by transduction of a viral vector.

The components used in the method of the present invention may be introduced to the cell or population of cells at the same time or in any order.

In some embodiments, the first component and the second component are introduced into the cell or the population of cells at the same time.

In some embodiments, the first component and the third component are introduced into the cell or the population of cells at the same time.

In some embodiments the second component is introduced into the cell or the population of cells about 1 day to about 14 days after the first component is introduced into the cell or the population of cells. Preferably the second component is introduced into the cell or the population of cells about 2 days to about 4 days after the first component is introduced into the cell or the population of cells.

First Component

The term “first component” as used herein refers to a donor reporter cassette comprising the nucleotide sequence encoding the selector and a nucleotide sequence of interest (NOI).

Donor Reporter Cassette

The term “donor reporter cassette” may be used interchangeably with the terms “donor vector”, “donor DNA vector”, “donor plasmid” and “donor cassette”.

In some embodiments, the donor reporter cassette sequentially comprises:
(i) a left homology arm (HA) comprising a nucleotide sequence homologous to a target locus;
(ii) the nucleotide sequence encoding the selector operably linked to a minimal promoter;
(iii) the NOI operably linked to a promoter; and
(iv) a right homology arm (HA) comprising a nucleotide sequence homologous to the target locus;

wherein the ETT polypeptide of the second component or the ETT polypeptide expressed by the second component activates the minimal promoter when the nucleotide sequence encoding the selector is inserted into the target locus. This donor reporter cassette is suitable for use in the SMArT method. This donor reporter cassette is suitable for inserting a NOI into a target locus and/or correcting or disrupting a NOI at a target locus.

In other embodiments, the donor reporter cassette sequentially comprises:
(i) a left homology arm (HA) comprising a nucleotide sequence homologous to a target locus;
(ii) optionally, a splicing acceptor site (SA);
(iii) the NOI;
(iv) the nucleotide sequence encoding the selector operably linked to a minimal promoter; and
(v) a right homology arm (HA) comprising a nucleotide sequence homologous to the target locus;

wherein the ETT polypeptide of the second component or the ETT polypeptide expressed by the second component activates the minimal promoter when the nucleotide sequence encoding the selector is inserted into the target locus. This donor reporter cassette is suitable for use in the SMArT method. This donor reporter cassette is suitable for inserting a NOI into a target locus and/or correcting or disrupting a NOI at a target locus.

In other embodiments, the donor reporter cassette sequentially comprises:
(i) a left homology arm (HA) comprising a nucleotide sequence homologous to a target locus;
(ii) optionally, a splicing acceptor site (SA);
(iii) the NOI;
(iv) optionally, a nucleotide sequence encoding a 2A self-cleaving peptide (2A) or an internal ribosome entry site (IRES) element;
(v) the nucleotide sequence encoding the selector, optionally the nucleotide sequence encoding the selector is operably linked to a minimal promoter; and
(vi) a right homology arm (HA) comprising a nucleotide sequence homologous to the target locus;

wherein the ETT polypeptide of the second component or the ETT polypeptide expressed by the second component activates an endogenous promoter in the target locus. This donor reporter cassette is suitable for use in the SMArTER method. This donor reporter cassette is suitable for inserting a NOI into a target locus and/or correcting or disrupting a NOI at a target locus.

Expression of a selector may be increased by use of a binding site for a translational activator and/or an enhancer.

In some embodiments, the donor reporter cassette further comprises at least one binding site for a translational activator such as a modular RNA activator containing the aptamer for eukaryotic initiation factor 4G (eIF4G). The at least one binding site for the translational activator may be inserted downstream and close to the transcriptional start site in order to boost mRNA translation.

In some embodiments, the donor reporter cassette further comprises an enhancer (such as EF1alpha promoter). The enhancer may be inserted close to the promoter.

In some embodiments, the translational activator and/or an enhancer may be inducible in the presence of a modulator. In other embodiments, the translational activator and/or an enhancer may be inducible in the absence of a modulator.

In some embodiments, the donor reporter cassette further comprises a regulatory element.

In some embodiments, the donor reporter cassette sequentially comprises:
(i) a left homology arm (HA) comprising a nucleotide sequence homologous to a target locus;
(ii) the NOI, optionally operably linked to a promoter;
(iii) the nucleotide sequence encoding the selector, wherein the nucleotide sequence encoding the selector is operably linked to a minimal promoter and the minimal promoter is operably linked to a regulatory element; and
(iv) a right homology arm (HA) comprising a nucleotide sequence homologous to the target locus;

wherein the ETT polypeptide of the second component or the ETT polypeptide expressed by the second component binds to the regulatory element and activates the minimal promoter when the nucleotide sequence encoding the selector is inserted into the target locus and when a modulator is present in the cell or population of cells. This donor reporter cassette is suitable for use in the SMArT-D method. This donor reporter cassette is suitable for inserting a NOI into a target locus and/or correcting or disrupting a NOI at a target locus.

In other embodiments, the donor reporter cassette sequentially comprises:
(i) a left homology arm (HA) comprising a nucleotide sequence homologous to a target locus;
(ii) the NOI, optionally operably linked to a promoter;
(iii) the nucleotide sequence encoding the selector, wherein the nucleotide sequence encoding the selector is operably linked to a minimal promoter and the minimal promoter is operably linked to a regulatory element; and
(iv) a right homology arm (HA) comprising a nucleotide sequence homologous to the target locus;

wherein the ETT polypeptide of the second component or the ETT polypeptide expressed by the second component binds to the regulatory element and activates the minimal promoter when the nucleotide sequence encoding the selector is inserted into the target locus and when a modulator is not present in the cell or population of cells. This donor reporter cassette is suitable for use in the SMArT-D method. This donor reporter cassette is suitable for inserting a NOI into a target locus and/or correcting or disrupting a NOI at a target locus.

In some embodiments, the donor reporter cassette further comprises a splicing acceptor site (SA).

In some embodiments, the 2A self-cleaving peptide (2A) has the sequence shown as SEQ ID NO: 55 or has at least 75%, 80%, 85%, 90%, 95%, 97%, 98% or 99% identity to SEQ ID NO: 55.

In some embodiments, the donor reporter cassette further comprises at least one insulator element.

The term “insulator element” as used herein refers to a nucleotide sequence which is capable of limiting the activity of an enhancer on a promoter.

The insulator element may be, for example, derived or derivable from CCCTC-binding factor (CTCF)-dependent or independent binding sites.

In some embodiments the donor reporter cassette comprises an SV40polyA sequence which has the sequence shown as SEQ ID NO: 51 or has at least 75%, 80%, 85%, 90%, 95%, 97%, 98% or 99% identity to SEQ ID NO: 51.

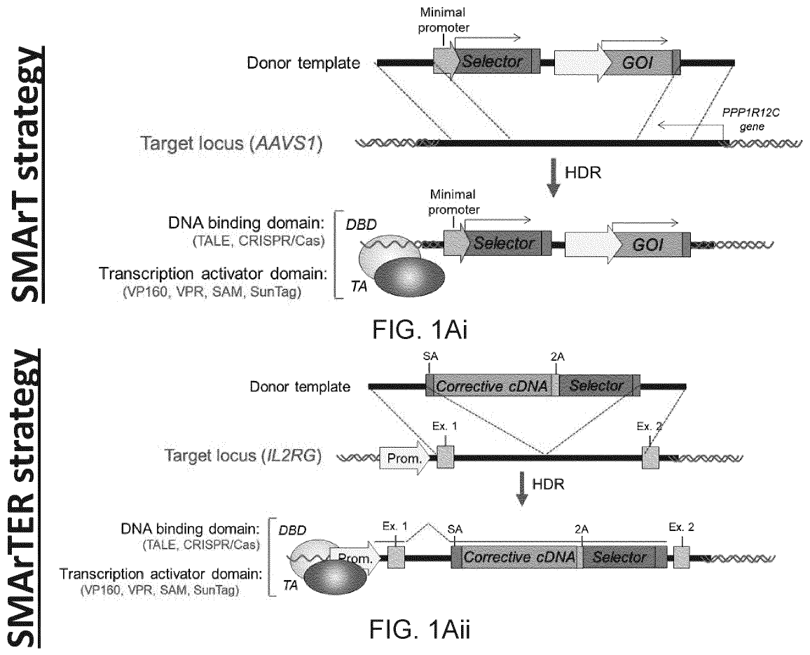

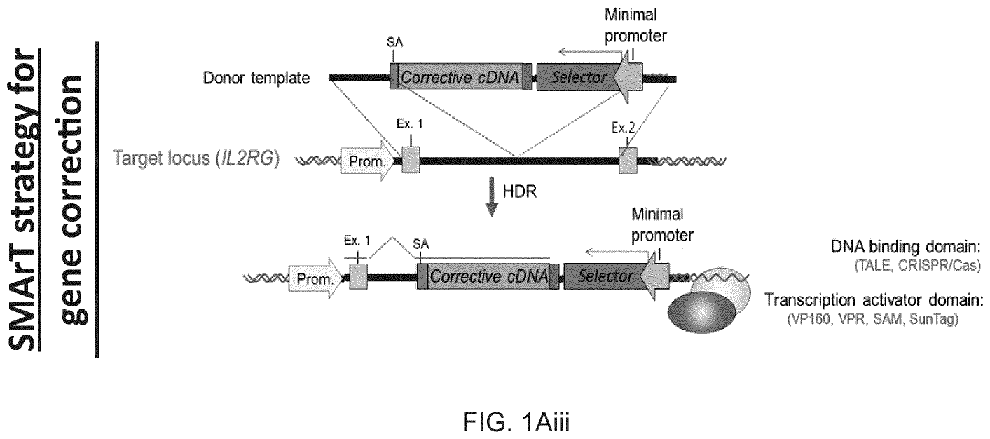

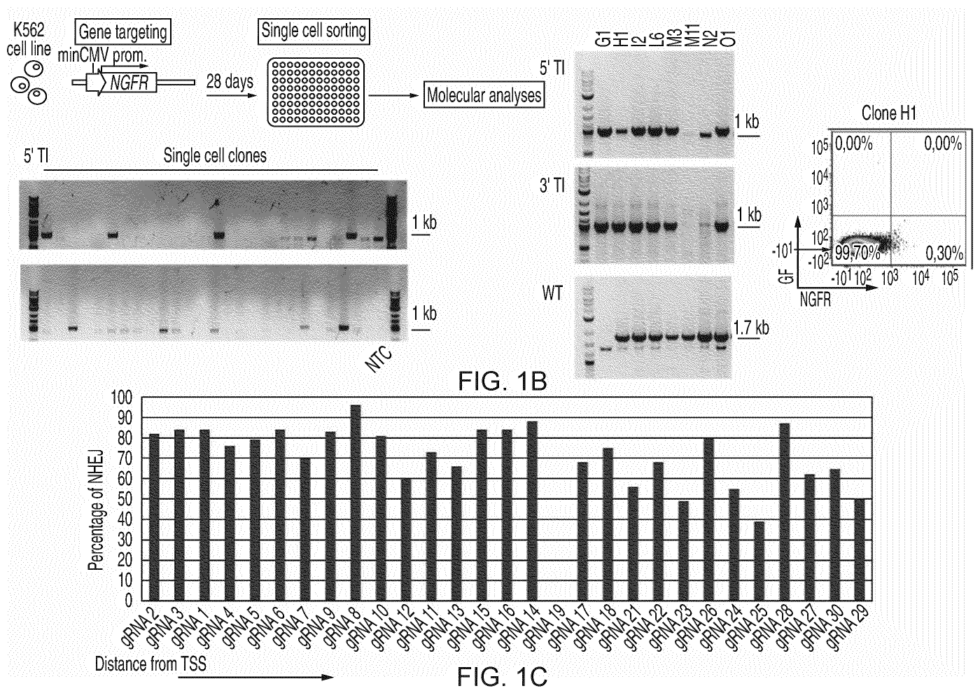

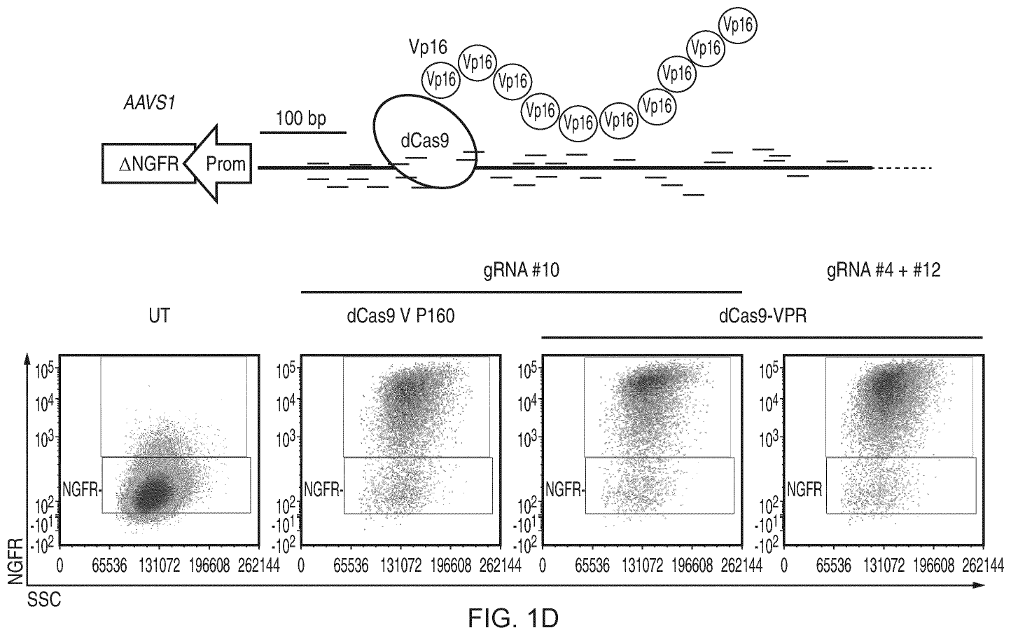

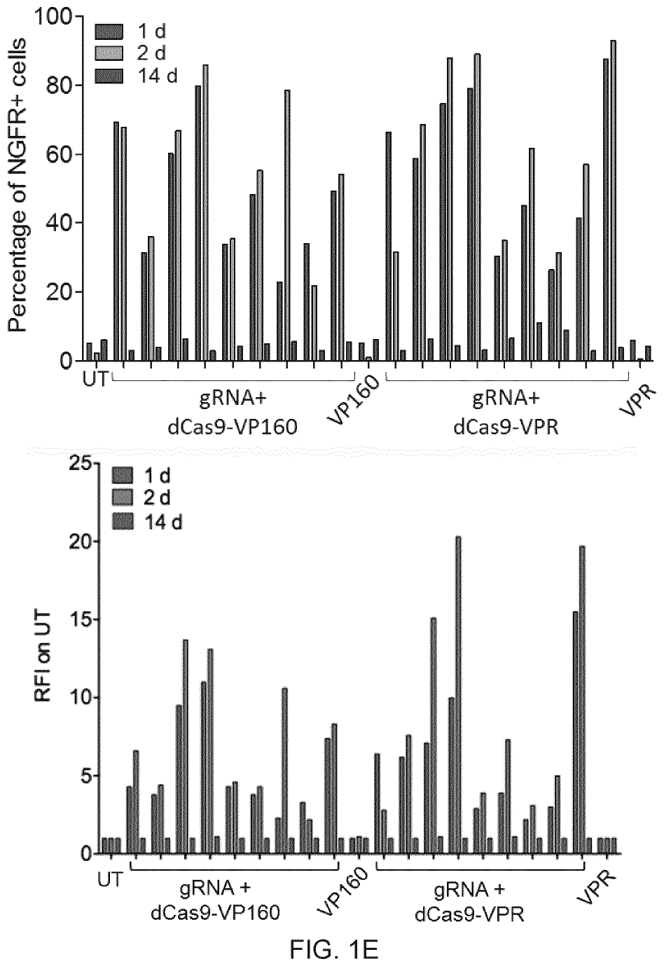

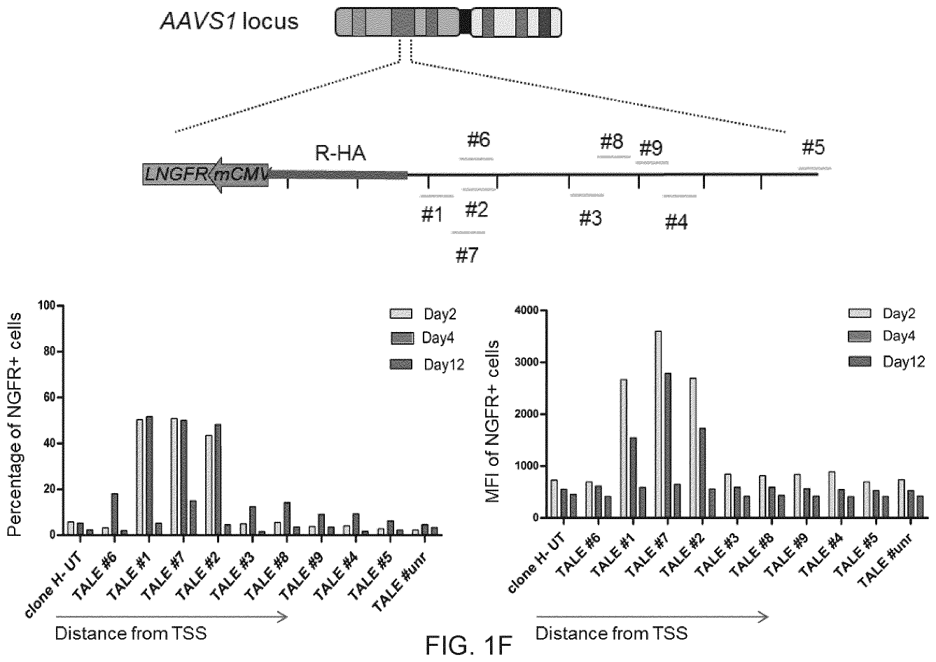

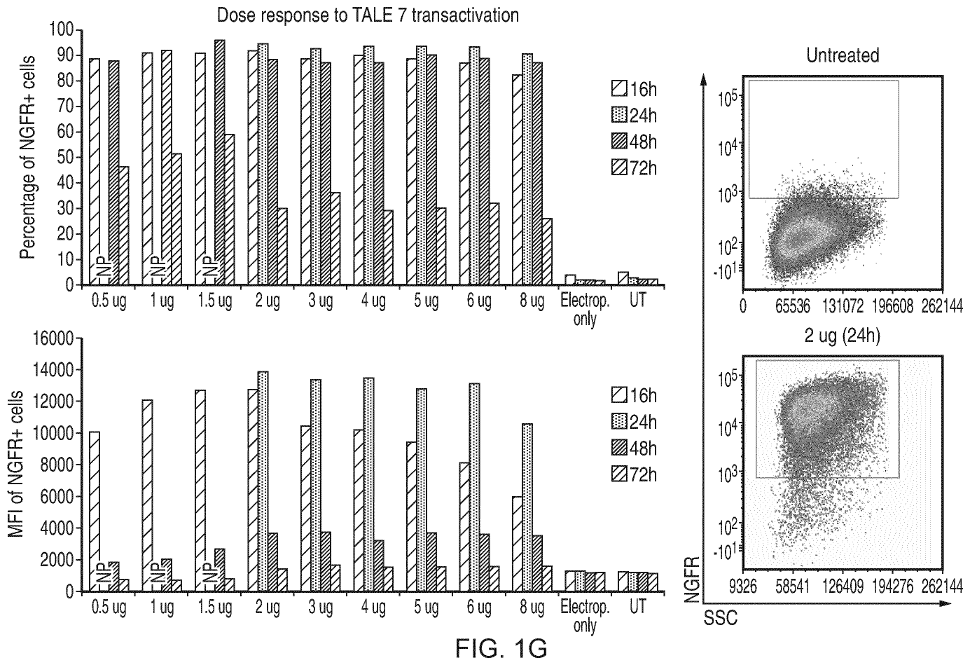

FIGS. 1 and 2.1 detail specific examples of donor cassettes for use in the method of the present invention.

In some embodiments, the donor reporter cassette is a plasmid.

In some embodiments, a vector comprises the donor reporter cassette.

In some embodiments, the vector is a plasmid. In other embodiments, the vector is a viral vector.

In some embodiments, the vector is an expression vector.

Homology Arms (HA)

The donor cassette comprises homology arms (HAs). Typically, the donor cassette comprises a left homology arm (left HA) and a right homology arm (right HA).

Each homology arm (HA) comprises a nucleotide sequence which is homologous to at least part of the target locus. Typically, the left homology arm has homology with a sequence at the 5′ end of the target locus and the right homology arm has homology with a sequence at the 3′ end of the target locus.

Homology-directed repair (HDR) is a process where a DNA double-strand break (DSB) (such as in a target locus) is repaired by homologous recombination using a DNA template. Homology driven repair (HDR) can be influenced by the length of the homology arms.

In some embodiments, the left homology arm is about 500 to about 1000 nucleotides in length.

In some embodiments, the left homology arm has the sequence shown as SEQ ID NO: 50 or has at least 75%, 80%, 85%, 90%, 95%, 97%, 98% or 99% identity to SEQ ID NO: 50.

In other embodiments, the left homology arm is between about 50 to about 500 nucleotides in length.

In other embodiments, the left homology arm is between about 50 to about 600 nucleotides in length.

In some embodiments the left homology arm is between about 80 to about 200 nucleotides in length. Preferably the left homology arm is between about 130 to about 170 nucleotides in length. Preferably the left homology arm is between about 140 to about 160 nucleotides in length. More preferably the left homology arm is about 150 nucleotides in length.

In some embodiments, the left HA has the sequence shown as SEQ ID NO: 52 or has at least 75%, 80%, 85%, 90%, 95%, 97%, 98% or 99% identity to SEQ ID NO: 52.

In some embodiments the left homology arm is between about 200 to about 350 nucleotides in length. Preferably the left homology arm is between about 250 to about 330 nucleotides in length. Preferably the left homology arm is between about 280 to about 310 nucleotides in length. More preferably the left homology arm is about 290 nucleotides in length.

In some embodiments, the left HA has the sequence shown as SEQ ID NO: 53 or has at least 75%, 80%, 85%, 90%, 95%, 97%, 98% or 99% identity to SEQ ID NO: 53.

In some embodiments, the left HA has the sequence shown as SEQ ID NO: 61 or has at least 75%, 80%, 85%, 90%, 95%, 97%, 98% or 99% identity to SEQ ID NO: 61.

In some embodiments, the left HA has the sequence shown as SEQ ID NO: 63 or has at least 75%, 80%, 85%, 90%, 95%, 97%, 98% or 99% identity to SEQ ID NO: 63.

In some embodiments, the right homology arm is about 500 to about 1000 nucleotides in length.

In other embodiments the right homology arm is between about 200 to about 300 nucleotides in length. Preferably the right homology arm is between about 250 to about 290 nucleotides in length. Preferably the right homology arm is between about 260 to about 280 nucleotides in length. More preferably the left homology arm is about 270 nucleotides in length.

In some embodiments, the right HA has the sequence shown as SEQ ID NO: 56 or has at least 75%, 80%, 85%, 90%, 95%, 97%, 98% or 99% identity to SEQ ID NO: 56.

In some embodiments, the right HA has the sequence shown as SEQ ID NO: 62 or has at least 75%, 80%, 85%, 90%, 95%, 97%, 98% or 99% identity to SEQ ID NO: 62.

In some embodiments, the right HA has the sequence shown as SEQ ID NO: 64 or has at least 75%, 80%, 85%, 90%, 95%, 97%, 98% or 99% identity to SEQ ID NO: 64.

Typically, inserts between the left homology arm and the right homology arm can be about 1000 to about 2000 nucleotides in length.

Target Locus

The term “target locus” as used herein may be used interchangeably with the term “target gene”. The target locus is a desired site in a genome for insertion (or integration), correction, mutation or deletion.

The target locus can be any locus in the genome of a cell.

In some embodiments, the target locus is a safe harbour. The term “safe harbour” as used herein refers to a location in the genome in which the integration of a nucleotide sequence does not disrupt any regulatory or coding sequence nor perturb the nearest regulatory elements or the transcriptional profiling of neighbouring genes. The term “safe harbour” may be used interchangeably with the terms “neutral area”, “neutral region” and “neutral gene”.

In some embodiments, the target locus is adeno-associated virus integration site 1 (AAVS1) or a common integration site (CIS) of lentiviral vectors.

In other embodiments, the target locus is IL2RG, gp91phox, HBB, RAG1, CD40LG, TRAC, TRBC, STAT or PRF1.

In other embodiments, the target locus is a gene encoding for a protein expressed in the skin such as collagen, keratin, laminin, desmocolin, desmoplachine, desmoglein, placoglobin, placophylline, integrin or other proteins that are involved in desmosomes and hemidesmosomes.

In some embodiments, the target locus is adeno-associated virus integration site 1 (AAVS1).

In other embodiments, the target locus is IL2RG.

In other embodiments, the target locus is CD40LG.

In some embodiments, the genome of the cell comprises at least one ETT binding site operably linked to the target locus.

In some embodiments, an endogenous promoter is operably linked to the at least one ETT binding site. In some embodiments, the ETT binding site is located downstream of the endogenous promoter. In other embodiments, the ETT binding site is located upstream of the endogenous promoter. Without wishing to be bound by theory, the ETT binding site acts as an enhancer.

In some embodiments, the ETT binding site comprises a nucleotide sequence having the sequence shown as any one of SEQ ID NOs 32 to 40 or a sequence having at least 75%, 80%, 85%, 90%, 95%, 97%, 98% or 99% identity thereto. Preferably the ETT binding site comprises a nucleotide sequence having the sequence shown as SEQ ID NO: 32, SEQ ID NO: 33, SEQ ID NO: 34 and SEQ ID NO: 38. More preferably the ETT binding site comprises a nucleotide sequence shown as SEQ ID NO: 34 or SEQ ID NO: 38.

In some embodiments, the ETT binding site comprises a nucleotide sequence encoding at least one tetO sequence.

In some embodiments, the ETT binding site comprises a nucleotide sequence having the sequence shown SEQ ID NO: 65 or a sequence having at least 75%, 80%, 85%, 90%, 95%, 97%, 98% or 99% identity thereto.

Selector

The terms “nucleotide sequence encoding the selector”, “reporter”, “reporter gene”, “marker” and “selector” may be used interchangeably. As used herein the term “nucleotide sequence encoding the selector” refers to any nucleotide sequence which can be used to select cells in which the nucleotide sequence has been inserted into the target locus (desired integration site) in the genome from cells in which the nucleotide sequence has not been inserted or in which the sequence is inserted into the wrong site.

In some embodiments, cells in which a selector has been inserted into the wrong site will not express the selector. In other embodiments, (such as SMArT-D) cells in which the selector is inserted into a non-target site will express the selector.

In some embodiments, the nucleotide sequence encoding the selector encodes a selector selected from the group consisting of: mutated low-affinity nerve growth factor receptor (ΔLNGFR); truncated NGFR; a drug-resistance protein (such as proteins having neomycin or puromycin resistance, or mutated methylguanine DNA methyltransferase (MGMT)); truncated cell surface proteins (such as CD19 and EGFR-PMID: 21653320, Wang et al Blood 118(5):1255-63); proteins that confer selective growth and/or engraftment advantage after in vivo transplantation of the genome edited cell (such as receptor tyrosine kinase (c-Kit), C-X-C chemokine receptor type 4 (CXCR4, or CXCR4 WHIM) and CD47) and reporter proteins (for example, fluorescent proteins such as eGFP).

In some embodiments, the nucleotide sequence encoding the selector encodes a nerve growth factor receptor such as a low-affinity nerve growth factor receptor or a mutated low-affinity nerve growth factor receptor (ΔLNGFR). Examples of nucleotide sequences encoding nerve growth factor receptor are SEQ ID NO: 44 and sequences having at least 75%, 80%, 85%, 90%, 95%, 97%, 98% or 99% identity thereto. Other Examples of nucleotide sequences encoding nerve growth factor receptor (NGFR) are SEQ ID NO: 67 and sequences having at least 75%, 80%, 85%, 90%, 95%, 97%, 98% or 99% identity thereto.

In other embodiments, the nucleotide sequence encoding the selector encodes a reporter protein such as eGFP. The term “eGFP” may be used herein interchangeably with the term “GFP”. Examples of nucleotide sequences encoding GFP are SEQ ID NO: 43 and sequences having at least 75%, 80%, 85%, 90%, 95%, 97%, 98% or 99% identity thereto.

In some embodiments, the nucleotide sequence encoding the selector is selected from the group consisting of SEQ ID NO: 43, SEQ ID NO: 44, SEQ ID NO: 67 and sequences having at least 75%, 80%, 85%, 90%, 95%, 97%, 98% or 99% identity thereto.

In some embodiments, the nucleotide sequence encoding the selector is fused to at least one nucleotide sequence encoding a destabilizer domain.

The term “destabilizer domain” as used herein refers to polypeptides capable of conferring instability to a polypeptide. For example, the destabilizer domains are able to introduce proteasomal degradation of the selector in the absence of specific stabilizer ligands.

In some embodiments, the destabilizer domain is a ligand-regulatable destabilizing domain. Examples of ligand-regulatable destabilizing domains include FKBP domains, bacterial DHFR and estrogen receptors (such as human estrogen receptors).

In some embodiments, the nucleotide sequence encoding the selector is operably linked to a promoter.

In some embodiments, the nucleotide sequence encoding the selector is operably linked to a minimal promoter.

The term “minimal promoter” as used herein refers to the minimal elements of a promoter, such the TATA box and transcription initiation site, which are inactive unless regulatory elements that enhance promoter activity are placed upstream.

In some embodiments, the minimal promoter is selected from the group consisting of synthetic promoter T6-SK and cytomegalovirus (CMV).

In some embodiments, the T6-SK promoter has the sequence shown as SEQ ID NO: 41 or a has at least 75%, 80%, 85%, 90%, 95%, 97%, 98% or 99% identity to the sequence shown as SEQ ID NO: 41.

In some embodiments, the CMV promoter has the sequence shown as SEQ ID NO: 42 or has at least 75%, 80%, 85%, 90%, 95%, 97%, 98% or 99% identity to SEQ ID NO: 42.

In some embodiments, the minimal promoter is operably linked to a regulatory element.

In some embodiments, the minimal promoter SK is operably linked to a regulatory element.

In some embodiments, the minimal promoter SK is operably linked to a regulatory element which has the sequence shown as SEQ ID NO: 41 or has at least 75%, 80%, 85%, 90%, 95%, 97%, 98% or 99% identity to the sequence shown as SEQ ID NO: 65.

In some embodiments, the minimal promoter SK is operably linked to a regulatory element which has the sequence shown as SEQ ID NO: 65.

The terms “T6-SK” and “S K” may be used interchangeably herein.

Regulatory Element

The term “regulatory element” as used herein refers to a nucleotide sequence which is capable of activating or enhancing the activity of a promoter.

Typically, at least one regulatory element is placed upstream of the promoter.

In other embodiments, the at least one regulatory element is placed elsewhere such as downstream of the promoter.

Examples of regulatory elements include translational activators and enhancers.

In some embodiments, the regulatory element activates or enhances the activity of a promoter.

In some embodiments, the regulatory element is inducible.

Among the inducible systems, the most extensively studied and tested is the TetO system, which allows to induce transient transactivation of the downstream gene (Gossen & Bujard, 1992). Tet-OFF systems consists of: i) Tet operon (TetO) derived from Escherichia coli, placed upstream of a minimal promoter, which can also be present in multiple copies; ii) the tetracycline transactivator (tTA) protein composed by the fusion of Tet repressor (TetR), derived from Escherichia coli, and multiple repeats of the activator domain VP16 from Herpes Simplex Virus. In the absence of tetracycline (or its derivatives as doxycycline), tTA binds TetO sequence(s) and induces transactivation of the gene under the control of the minimal promoter. The system can be turned off when tetracycline or its derivatives bind tTA, thus preventing tTA tethering on TetO. In Tet-ON systems the rtTA is able to bind TetO sequence(s) only in presence of tetracycline or derivatives.

Without wishing to be bound by theory, the ETT polypeptide binds to the regulatory element and this binding activates or enhances the activity of the promoter.

In some embodiments, the regulatory element comprises at least one tetracyclin operator (TetO) sequence.

In some embodiments, the regulatory element comprises at least 2, 3, 4, 5, 6, 7 or 8 TetO sequences. In some embodiments, the regulatory element comprises at least 2 TetO sequences. In other embodiments, the regulatory element comprises at least 7 TetO sequences.

In one embodiment, the regulatory element comprises 2 TetO sequences. This may be referred to as TetO2.

In another embodiment, the regulatory element comprises 7 TetO sequences. This may be referred to as TetO7.

Without wishing to be bound by theory, the binding of several tTA or rtTAs to the TetO sequences repeats enhances transactivation of the selector which, in turn, enhances the selection of the genome edited cells.

Typically the TetO sequence (TCCCTATCAGTGATAGAGA) (SEQ ID NO: 59) separated by spacer sequences (for example: ACGATGTCGAGTTTAC) (SEQ ID NO: 60).

In some embodiments, the TetO sequence has the sequence TCCCTATCAGTGATAGAGA.

In some embodiments, the TetO sequence has at least 75%, 80%, 85%, 90%, 95%, 97%, 98% or 99% identity to the sequence TCCCTATCAGTGATAGAGA (SEQ ID NO: 59).

In some embodiments, the TetO sequence has the sequence shown as SEQ ID NO: 65 or has at least 75%, 80%, 85%, 90%, 95%, 97%, 98% or 99% identity to the nucleotide sequence shown as SEQ ID NO: 65.

In some embodiments, the TetO7 sequence has the sequence shown as SEQ ID NO: 65 or has at least 75%, 80%, 85%, 90%, 95%, 97%, 98% or 99% identity to the nucleotide sequence shown as SEQ ID NO: 65.

In some embodiments, the TetO sequence has the sequence CCCTATCAGMATAGAGA (SEQ ID NO: 66).

In some embodiments, the TetO sequence has at least 75%, 80%, 85%, 90%, 95%, 97%, 98% or 99% identity to the sequence CCCTATCAGTGATAGAGA (SEQ ID NO: 66).

In some embodiments, the TetO sequence has the sequence shown as SEQ ID NO: 76 or has at least 75%, 80%, 85%, 90%, 95%, 97%, 98% or 99% identity to the nucleotide sequence shown as SEQ ID NO: 76.

In some embodiments, the TetO7 sequence has the sequence shown as SEQ ID NO: 76 or has at least 75%, 80%, 85%, 90%, 95%, 97%, 98% or 99% identity to the nucleotide sequence shown as SEQ ID NO: 76.

In some embodiments, the ETT polypeptide expressed by the second component or the ETT polypeptide of the second component which binds to the regulator element is tetracyclin transactivator (tTA).

The tetracyclin transactivator (tTA) is capable of binding to the TetO sequence.

The tetracyclin transactivator (tTA) as used herein comprises the DNA binding domain (DBD) TetR and the transcriptional activator (TA) domain VP16.

An example of tetracyclin transactivator (tTA) is shown in SEQ ID NO: 58.

In some embodiments, the nucleotide sequence encoding tetracyclin transactivator (tTA) has the sequence shown as SEQ ID NO: 58 or has at least 75%, 80%, 85%, 90%, 95%, 97%, 98% or 99% identity to the nucleotide sequence shown as SEQ ID NO: 58.

In some embodiments, the nucleotide sequence encoding tetR has the sequence shown as SEQ ID NO: 74 or has at least 75%, 80%, 85%, 90%, 95%, 97%, 98% or 99% identity to the nucleotide sequence shown as SEQ ID NO: 74.

In some embodiments, the nucleotide sequence encoding VP16 has the sequence shown as SEQ ID NO: 75 or has at least 75%, 80%, 85%, 90%, 95%, 97%, 98% or 99% identity to the nucleotide sequence shown as SEQ ID NO: 75.

In other embodiments, the ETT polypeptide expressed by the second component or the ETT polypeptide of the second component which binds to the regulator element is reverse-tTA (rtTA).

In some embodiments, reverse-tTA (rtTA) has the sequence:

(SEQ ID NO: 70)
Atgtctagactggacaagagcaaagtcataaactctgctctggaattact
caatggagtcggtatcgaaggcctgacgacaaggaaactcgctcaaaagc
tgggagttgagcagcctaccctgtactggcacgtgaagaacaagcgggcc
ctgctcgatgccctgccaatcgagatgctggacaggcatcatacccactt
ctgccccctggaaggcgagtcatggcaagactttctgcggaacaacgcca
agtcattccgctgtgctctcctctcacatcgcgacggggctaaagtgcat
ctcggcacccgcccaacagagaaacagtacgaaaccctggaaaatcagct
cgcgttcctgtgtcagcaaggcttctccctggagaacgcactgtacgctc
tgtccgccgtgggccactttacactgggctgcgtattggaggaacaggag
catcaagtagcaaaagaggaaagagagacacctaccaccgattctatgcc
cccacttctgagacaagcaattgagctgttcgaccggcagggagccgaac
ctgccttccttttcggcctggaactaatcatatgtggcctggagaaacag
ctaaagtgcgaaagcggcgggccggccgacgcccttgacgattttgactt
agacatgctcccagccgatgcccttgacgactttgaccttgatatgctgc
ctgctgacgctcttgacgattttgaccttgacatgctccccggg

In some embodiments, reverse-tTA (rtTA) has at least 75%, 80%, 85%, 90%, 95%, 97%, 98% or 99% identity to the nucleotide sequence shown as SEQ ID NO: 70.

In some embodiments, reverse-tTA (rtTA) has the sequence:

(SEQ ID NO: 70)
Atgtctagactggacaagagcaaagtcataaactctgctctggaattact
caatggagtcggtatcgaaggcctgacgacaaggaaactcgctcaaaagc
tgggagttgagcagcctaccctgtactggcacgtgaagaacaagcgggcc
ctgctcgatgccctgccaatcgagatgctggacaggcatcatacccactt
ctgccccctggaaggcgagtcatggcaagactttctgcggaacaacgcca
agtcattccgctgtgctctcctctcacatcgcgacggggctaaagtgcat
ctcggcacccgcccaacagagaaacagtacgaaaccctggaaaatcagct
cgcgttcctgtgtcagcaaggcttctccctggagaacgcactgtacgctc
tgtccgccgtgggccactttacactgggctgcgtattggaggaacaggag
catcaagtagcaaaagaggaaagagagacacctaccaccgattctatgcc
cccacttctgagacaagcaattgagctgttcgaccggcagggagccgaac
ctgccttccttttcggcctggaactaatcatatgtggcctggagaaacag
ctaaagtgcgaaagcggcgggccggccgacgcccttgacgattttgactt
agacatgctcccagccgatgcccttgacgactttgaccttgatatgctgc
ctgctgacgctcttgacgattttgaccttgacatgctccccggg

In some embodiments, reverse-tTA (rtTA) has at least 75%, 80%, 85%, 90%, 95%, 97%, 98% or 99% identity to the nucleotide sequence shown as SEQ ID NO: 70.

The reverse tetracyclin transactivator (rtTA) is capable of binding to the TetO sequence when a modulator (such as a tetracycline (Tc) or a tetracycline derivative (e.g. deoxycycline)) is bound to the rtTA.

In the SMArT-D strategy the transactivator might not activate selector expression exclusively from the integrated donor reporter cassette but also from the unintegrated donor reporter donor, if present. However, tTA activity may be modified (up to complete inhibition) by a modulator (such as tetracycline or doxycycline treatment), which could enable upregulated or exclusive expression of the selector from cells in which the donor reporter cassette is integrated.

Advantageously, the SMArT-D strategy can be used with any target locus of interest without the need to design and screen gene-specific transactivators.

In some embodiments, binding of tTA or rtTA occurs close to the transcriptional start site (TSS). Without wishing to be bound by theory, the binding of tTA or rtTA close to the transcriptional start site (TSS) improves transactivation efficiency.

Modulator

The term “modulator” as used herein refers to any composition capable of binding to the ETT polypeptide, in particular the DNA binding domain (DBD), which modulates the activity, in particular DNA binding activity, of the ETT polypeptide

Examples of modulators of tetracyclin transactivator (tTA) and reverse tetracyclin transactivator (rtTA) are tetracyclines (Tc) and tetracycline derivatives such as deoxycycline (dox).

In one embodiment, the modulator is selected from the group consisting of tetracyclines (Tc), tetracycline derivatives (such as deoxycycline (dox)) and combinations thereof.

In one embodiment, the modulator is a tetracycline derivative.

In one embodiment, the modulator is deoxycycline.

Without wishing to be bound by theory, the binding of a modulator (such as tetracycline (Tc) or a tetracycline derivatives (such as dox) induces conformational changes in the DBD (such as the TetR domain) that prevents binding to the regulatory element (such as binding to the tetO sequence) if the donor reporter cassette has not been inserted into the genome; consequently the selector is not expressed. Conversely, cells in which the donor cassette has been inserted are still able to express the selector. Thus cells can be selected in which the donor cassette has been inserted.

Without wishing to be bound by theory, the binding of a modulator (such as tetracycline (Tc) or a tetracycline derivatives (such as dox) induces conformational changes in the DBD (such as reverse-TetR domain) that enables the DBD to bind to the regulatory element (such as the tetO sequence) when the donor reporter cassette has been inserted into the genome; consequently the selector is expressed. Conversely, cells in which the donor cassette has been inserted do express the selector. Thus cells can be selected in which the donor cassette has been inserted.

The modulator may be added to the cell or population of cells before, or after, or at the same time as the first component and/or second component and/or third component are introduced into the cell or population of cells.

In some embodiments, the modulator is added to the cell or population of cells about 6 hours to about 36 hours, about 6 hours to about 24 hours, or about 6 hours to about 12 hours after, the first component and/or second component and/or third component are introduced into the cell or population of cells.

The optimal dosage of a modulator may depend on multiple factors such as the donor reporter cassette, the target locus, and the nature of the cell or population of cells. The skilled person can readily determine an appropriate dose of the modulator (e.g. dox) to administer to the cell or population of cells.

In some aspect, the modulator (e.g. dox) is administered to the cell or population of cells so that about 10 nM to about 2 mM, or about 50 nM to about 1 mM, or about 80 nM to about 800 μM, or about 100 nM to 500 μM of the modulator is present in the media.

For example, the modulator (e.g. dox) is administered to the cell or population of cells so that at least 10 nM, at least 25 nM, at least 50 nM, at least 80 nM, at least 100 nM, at least 200 nM, at least 300 nM, at least 400 nM, at least 500 nM, at least 100 mM, or at least 200 mM of the modulator is present in the media.

The skilled person can readily determine when to wash the cell or population of cells treated with the modulator. This washing may be referred to as modulator (e.g. dox) withdrawal or washout.

The cell or population of cells treated with a modulator may be washed at least once, at least twice, at least thrice with a buffer solution (e.g. PBS) or a media which is suitable for maintaining and/or growing the cell or population of cells.

The cell or population of cells may be washed at least 6 hours, at least 12 hours, at least 18 hours, at least 21 hours, at least 24 hours, at least 30 hours, at least 36 hours, at least 48 hours, at least 60 hours, or at least 72 hours after treatment with a modulator and/or introduction of the first component and/or second component and/or third component into the cell or population of cells.

The edited cell or population of edited cells may be exposed to the modulator for at least 6 hours, at least 12 hours, at least 18 hours, at least 21 hours, at least 24 hours, at least 30 hours, at least 36 hours, at least 48 hours, at least 60 hours, or at least 72 hours after treatment the introduction of the first component and/or second component and/or third component into the cell or population of cells.

NOI

The term “NOI” may be used interchangeably with the term “GOI”, “gene of interest” or “corrective cDNA”.

In some embodiments, the NOI is IL2RG, gp91phox, HBB, RAG1, CD40LG, TRAC, TRBC, STAT or PRF1.

In other embodiments, the NOI is a gene encoding for a protein expressed in the skin such as collagen, keratin, laminin, desmocolin, desmoplachine, desmoglein, placoglobin, placophylline, integrin or other proteins that are involved in desmosomes and hemidesmosomes.

In some embodiments, the NOI is IR2RG. Examples of NOI encoding IR2LG are SEQ ID NO: 54 and sequences having at least 75%, 80%, 85%, 90%, 95%, 97%, 98% or 99% identity thereto.

In some embodiments, the NOI is selected from the group consisting of SEQ ID NO: 54 and sequences having at least 75%, 80%, 85%, 90%, 95%, 97%, 98% or 99% identity thereto.

In some embodiments, the NOI is operably linked to a promoter.

In some embodiments, the nucleotide sequence encoding the selector and the NOI may be the same.

The term “transcription starting site (TSS)” as used herein refers to the first nucleotide where RNA polymerase begins to synthesize the RNA transcript.

In some embodiments, the NOI is about 50 to about 400 nucleotides downstream of a transcription starting site (TSS). Preferably the NOI about 100 to about 250 nucleotides downstream of the TSS.

Second Component

The term “second component” as used herein refers to an engineered transcriptional transactivator (ETT) polypeptide or a nucleotide sequence encoding an ETT polypeptide.

The term “ETT polypeptide” as used herein refers to a polypeptide comprising a DNA binding domain (DBD) and at least one transcription activator (TA) domain.

The ETT polypeptide of the second component or the ETT polypeptide expressed by the second component binds to an ETT binding site in the genome of the cell.

In one embodiment, the ETT binding site is a regulatory element.

In one embodiment, the ETT binding site is one or more tetO sequences.

In some embodiments, the ETT polypeptide of the second component or the ETT polypeptide expressed by the second component activates the minimal promoter when the nucleotide sequence encoding the selector is inserted into the target locus. Typically this results in expression of the selector.

In other embodiments, the ETT polypeptide of the second component or the ETT polypeptide expressed by the second component activates an endogenous promoter in the target locus. Typically, when a nucleotide sequence encoding the selector is inserted into the target locus the selector is expressed.

In some embodiments, the nucleotide sequence encoding an ETT polypeptide is a plasmid.

In some embodiments, a vector comprises the nucleotide sequence encoding an ETT polypeptide.

In some embodiments, the vector is a plasmid. In other embodiments, the vector is a viral vector.

In some embodiments, the vector is an expression vector.

DNA Binding Domain (DBD)

DNA binding domains (DBDs) contain at least one structural motif that recognizes double- or single-stranded DNA.

Examples of DBD for use in the present invention include, but are not limited to, transcriptional activator-Like effector (TALE) DBDs (such as TALE7 DBD and TALE3 DBD), zinc fingers (ZNF), catalytically inactive Cpf1 and catalytically inactive Cas (dCas) (such as dCas9).

Additional examples of DBD for use in the present invention include TetR and reverseTetR.

Catalytically inactive Cas variants (such as dCas9) have been isolated from various bacteria (such as S. aureus, S. thermophilus, and N. meningitidis)—see Ran et al., Nature 2015 (PMID: 25830891), Lee et al., Mol Ther 2016 (PMID: 26782639)). Zetsche et al., (Cell 2015 pii: S0092-8674(15)01200-3 (PMID: 26422227)) discloses other Cas protein (such as Cpf1).

In some embodiments, the DBD is a TALE.

In some embodiments, the DBD is a catalytically inactive Cas (dCas). Preferably, the dCas is dCas9.

In some embodiments the DBD is tTA.

In other embodiments, the DBD is rtTA.

In some embodiments, the DBD is encoded by a nucleotide sequence selected from the group consisting of SEQ ID NO: 45, SEQ ID NO: 48, SEQ ID NO: 49 and sequences having at least 75%, 80%, 85%, 90%, 95%, 97%, 98% or 99% identity thereto.

In some embodiments, the DBD is capable of binding to one or more of the nucleotide sequences selected from the group consisting of SEQ ID NOs 32 to 40 and sequences having at least 75%, 80%, 85%, 90%, 95%, 97%, 98% or 99% identity thereto. In some embodiments, the DBD is capable of binding to one or more nucleotide sequences selected from the group consisting of SEQ ID NO: 32, SEQ ID NO: 33, SEQ ID NO: 34 and SEQ ID NO: 38. Preferably, the DBD is capable of binding to SEQ ID NO: 34 or SEQ ID NO: 38.

Transcription Activator (TA) Domain

Transcription activator (TA) domains contain binding sites for polypeptides which activate transcription.

Examples of TA domains for use in the present invention include, but are not limited to, VP16, VP64, VP128, VP160, VPR, p65, Rta, HSF1, synergistic activation mediator (SAM), and SunTag.

In some embodiments, the TA is VPR. In other embodiments, the TA is VP160. In other embodiments, the TA is VP16.

In some embodiments, the TA domain is encoded by a nucleotide sequence selected from the group consisting of SEQ ID NO: 46, SEQ ID NO: 47, and sequences having at least 75%, 80%, 85%, 90%, 95%, 97%, 98% or 99% identity thereto.

In some embodiments, the second component is an engineered transcriptional transactivator (ETT) polypeptide or a nucleotide sequence encoding an ETT polypeptide; wherein the ETT polypeptide comprises a DNA binding domain (DBD) and at least one transcription activator (TA) domain;
wherein the DBD is selected from the group consisting of a Transcriptional Activator-Like Effector (TALE) DBD, a Zinc finger, catalytically inactive Cpf1 or catalytically inactive Cas (dCas), and
the TA domain is selected from the group consisting of VP16, VP64, VP128, VP160, VPR, p65, Rta, HSF1, SAM, and SunTag.

In other embodiments, the second component is an engineered transcriptional transactivator (ETT) polypeptide or a nucleotide sequence encoding an ETT polypeptide; wherein the ETT polypeptide comprises a DNA binding domain (DBD) and at least one transcription activator (TA) domain;
wherein the DBD is selected from the group consisting TetR or reverseTetR (rTetR), and
the TA domain is selected from the group consisting of VP16, VP64, VP128, VP160, VPR, p65, Rta, HSF1, SAM, and SunTag.

In some embodiments, the second component is an engineered transcriptional transactivator (ETT) polypeptide or a nucleotide sequence encoding an ETT polypeptide; wherein the ETT polypeptide comprises TetR and at least one VP16.

In some embodiments, the second component is a nucleotide sequence encoding an ETT polypeptide wherein the nucleotide sequence has the sequence shown as SEQ ID NO: 58 or has at least 75%, 80%, 85%, 90%, 95%, 97%, 98% or 99% identity to the nucleotide sequence shown as SEQ ID NO: 58.

In some embodiments, the second component is an engineered transcriptional transactivator (ETT) polypeptide or a nucleotide sequence encoding an ETT polypeptide; wherein the ETT polypeptide comprises reverse-TetR and at least one VP16.

Third Component

The term “third component” as used herein refers to a nuclease system comprising a genome targeted nuclease. The nuclease system cuts and/or repairs genomic DNA. The presence of the nuclease system in a cell or a population of cells enables the insertion of the nucleotide sequence encoding the selector (and, optionally, a minimal promoter) and the correction or insertion or deletion or mutation of a NOI in the target locus.

The genome targeted nuclease is provided as a protein, RNA, DNA, or an expression vector comprising a nucleic acid sequence that encodes the genome targeted nuclease.

In some embodiments, the expression vector is a plasmid. In other embodiments, the expression vector is a viral vector.

In some embodiments, the genome targeted nuclease is a transcriptional activator-like effector nuclease (TALEN), a zinc finger nuclease (ZNF), a CRISPR-Cas (such as CRISPR-Cas9, or SpCas9, or CRISPR-Cpf (such as CRISPR-Cpf1)) or a meganuclease.

In some embodiments, the third component additionally comprises a guide RNA comprising at least one targeted genomic sequence. Guide RNA comprising at least one targeted genomic sequence is capable of binding to at least one sequence in the genome of a cell.

Typically, the third component additionally comprises a guide RNA when the genome targeted nuclease is a CRISPR-Cas (such as CRISPR-Cas9 or CRISPR-Cpf (such as CRISPR-Cpf1)). Typically the guide RNA is delivered simultaneously with the genome targeted nuclease. In some embodiments, the guide RNA is delivered before or after the genome targeted nuclease.

The guide RNA (gRNA) is provided as an RNA molecule, DNA molecule, or an expression vector comprising a nucleic acid sequence that encodes the gRNA.

In some embodiments, the expression vector is a plasmid. In other embodiments, the expression vector is a viral vector.

In some embodiments, the gRNA is capable of binding to the target locus AAVS1.

In some embodiments, the gRNA is capable of binding to the target locus IL2RG.

In some embodiments, the gRNA is capable of binding to the target locus CD40LG.

Typically, the gRNA binds upstream of the promoter.

In some embodiments, the gRNA is capable of binding to the nucleotide sequence

| (SEQ ID NO: 71) | |
|---|---|
| | GTCACCAATCCTGTCCCTAGTGG. |

In other embodiments, the gRNA is capable of binding to the nucleotide sequence

| (SEQ ID NO: 72) | |
|---|---|
| | ACTGGCCATTACAATCATGTGGG. |

In other embodiments, the gRNA is capable of binding to the nucleotide sequence

| (SEQ ID NO: 73) | |
|---|---|
| | TGGATGATTGCACTTTATCAGGG. |

In some embodiments the gRNA is capable of binding to one or more of the nucleotide sequences selected from the group consisting of SEQ ID NOs 1 to 31 and sequences having at least 75%, 80%, 85%, 90%, 95%, 97%, 98% or 99% identity thereto. Suitably the gRNA is capable of binding to one or more of the nucleotide sequences selected from the group consisting of SEQ ID NOs 1 to 18 and 21 to 31 and sequences having at least 75%, 80%, 85%, 90%, 95%, 97%, 98% or 99% identity thereto. Preferably the gRNA is capable of binding to one or more nucleotide sequences selected from the group consisting of SEQ ID NO: 4, SEQ ID NO: 9, SEQ ID NO: 10 and SEQ ID NO: 12.

In some embodiments the gRNA is capable of binding to one or more of the nucleotide sequences selected from the group consisting of SEQ ID NOs: 70 to 72 and sequences having at least 75%, 80%, 85%, 90%, 95%, 97%, 98% or 99% identity thereto.

In some embodiments, the guide RNA (gRNA) is provided as a single guide RNA capable of binding to one nucleotide sequence. In other embodiments, the guide RNA is provided as a combination of two types of guide RNA each capable of binding to a different nucleotide sequence. For example, the guide RNA is provided as a combination of two types of guide RNA of which one is capable of binding to SEQ ID NO: 4 and the other is capable of binding to SEQ ID NO: 12. In other embodiments, the guide RNA is provided as a combination of three types of guide RNA each capable of binding to a different nucleotide sequence. In other embodiments, the guide RNA is provided as a combination of four types of guide RNA each capable of binding to a different nucleotide sequence. In other embodiments, the guide RNA is provided as a combination of at least five types of guide RNA each capable of binding to a different nucleotide sequence.

In some embodiments, the nuclease system is in the form of ribonucleoprotein (RNP).

In some embodiments, the nuclease system is an RNP comprising CRISPR-Cas. For example, the nuclease system is an RNP comprising CRISPR-Cas and cr:tracrRNA (Integrated DNA technologies).

Preferably, the third component is a nuclease system comprising CRISPR-Cas and a guide RNA.

Preferably, the third component is a nuclease system comprising CRISPR-Cpf and a guide RNA.

Variants

In addition to the specific polypeptides and polynucleotides mentioned herein, the present invention also encompasses the use of variants.

Variant sequences of SEQ ID NOs recited herein may have at least 75%, 80%, 85%, 90%, 95%, 97%, 98% or 99% sequence identity to the reference sequence SEQ ID NOs. Preferably, the variant sequence retains one or more functions of the reference sequence (i.e. is a functional variant).

Variant sequences may comprise substitutions, additions, deletions and/or insertions.

Variant sequences may comprise one or more conservative substitutions. Conservative amino acid substitutions may be made on the basis of similarity in polarity, charge, solubility, hydrophobicity, hydrophilicity, and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

Conservative substitutions may be made, for example according to the Table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

The present invention also encompasses homologous substitution (substitution and replacement are both used herein to mean the interchange of an existing amino acid residue, with an alternative residue) variants i.e. like-for-like substitution such as basic for basic, acidic for acidic, polar for polar etc.

Unless otherwise explicitly stated herein by way of reference to a specific, individual amino acid, amino acids may be substituted using conservative substitutions as recited below.

An aliphatic, non-polar amino acid may be a glycine, alanine, proline, isoleucine, leucine or valine residue.

An aliphatic, polar uncharged amino may be a cysteine, serine, threonine, methionine, asparagine or glutamine residue.

An aliphatic, polar charged amino acid may be an aspartic acid, glutamic acid, lysine or arginine residue.

An aromatic amino acid may be a histidine, phenylalanine, tryptophan or tyrosine residue.

Suitably, a conservative substitution may be made between amino acids in the same line in the Table above.

In some embodiments, the sequence is codon optimized for the subject.

Vectors

In some embodiments, a vector comprises the first component (i.e. the vector comprises the donor cassette).

In some embodiments, a vector comprises a nucleotide sequence encoding an ETT polypeptide.

In some embodiments, a vector comprises a nucleotide sequence encoding a nuclease.

A vector is a tool that allows or facilitates the transfer of an entity from one environment to another.

The vector may be single-stranded or double-stranded.

The vector may be an integrase-deficient vector such as a integrase-deficient lentiviral vector (IDLV).

In some embodiments, the vectors used in the present invention are plasmids. In other embodiments, the vectors used in the present invention are viral vectors.

In some embodiments, the viral vector is in the form of a viral vector particle.

In some embodiments, the components are a mixture of different types of vectors. For example, the first component is in the form of a viral vector, the second component is in the form of a plasmid vector and the third component is in the form of a plasmid vector

The viral vector may be, for example, an adeno-associated viral (AAV), adenoviral, a retroviral or lentiviral vector. Preferably, the viral vector is an AAV vector or a retroviral or lentiviral vector, more preferably an AAV vector.

By “vector derived from” a certain type of virus, it is to be understood that the vector comprises at least one component part derivable from that type of virus.

Adeno-Associated Viral (AAV) Vectors

Adeno-associated virus (AAV) is an attractive vector system for use in the invention as it has a high frequency of integration and it can infect non-dividing cells. This makes it useful for delivery of genes into mammalian cells in tissue culture.

AAV has a broad host range for infectivity. Details concerning the generation and use of AAV vectors are described in U.S. Pat. Nos. 5,139,941 and 4,797,368.

Recombinant AAV vectors have been used successfully for in vitro and in vivo transduction of marker genes and genes involved in human diseases.

Preferred vectors are those which are able to achieve a high transduction efficiency in human primary cells, such as HSPC cells.

In some embodiments, the vector is an AAV6 vector or a vector derived from an AAV6 vector. Preferably the vector is an AAV6 vector.

Adenoviral Vectors

The adenovirus is a double-stranded, linear DNA virus that does not go through an RNA intermediate. There are over 50 different human serotypes of adenovirus divided into 6 subgroups based on the genetic sequence homology. The natural targets of adenovirus are the respiratory and gastrointestinal epithelia, generally giving rise to only mild symptoms. Serotypes 2 and 5 (with 95% sequence homology) are most commonly used in adenoviral vector systems and are normally associated with upper respiratory tract infections in the young.

Adenoviruses have been used as vectors for gene therapy and for expression of heterologous genes. The large (36 kb) genome can accommodate up to 8 kb of foreign insert DNA and is able to replicate efficiently in complementing cell lines to produce very high titres of up to 10¹². Adenovirus is thus one of the best systems to study the expression of genes in primary non-replicative cells.

The expression of viral or foreign genes from the adenovirus genome does not require a replicating cell. Adenoviral vectors enter cells by receptor mediated endocytosis. Once inside the cell, adenovirus vectors rarely integrate into the host chromosome. Instead, they function episomally (independently from the host genome) as a linear genome in the host nucleus. Hence the use of recombinant adenovirus alleviates the problems associated with random integration into the host genome.

Retroviral and Lentiviral Vectors

A retroviral vector may be derived from or may be derivable from any suitable retrovirus. A large number of different retroviruses have been identified. Examples include murine leukaemia virus (MLV), human T-cell leukaemia virus (HTLV), mouse mammary tumour virus (MMTV), Rous sarcoma virus (RSV), Fujinami sarcoma virus (FuSV), Moloney murine leukaemia virus (Mo-MLV), FBR murine osteosarcoma virus (FBR MSV), Moloney murine sarcoma virus (Mo-MSV), Abelson murine leukaemia virus (A-MLV), avian myelocytomatosis virus-29 (MC29) and avian erythroblastosis virus (AEV). A detailed list of retroviruses may be found in Coffin, J. M. et al. (1997) Retroviruses, Cold Spring Harbour Laboratory Press, 758-63.

Retroviruses may be broadly divided into two categories, “simple” and “complex”. Retroviruses may be even further divided into seven groups. Five of these groups represent retroviruses with oncogenic potential. The remaining two groups are the lentiviruses and the spumaviruses. A review of these retroviruses is presented in Coffin, J. M. et al. (1997) Retroviruses, Cold Spring Harbour Laboratory Press, 758-63.

The basic structure of retrovirus and lentivirus genomes share many common features such as a 5′ LTR and a 3′ LTR. Between or within these are located a packaging signal to enable the genome to be packaged, a primer binding site, integration sites to enable integration into a host cell genome, and gag, pol and env genes encoding the packaging components—these are polypeptides required for the assembly of viral particles. Lentiviruses have additional features, such as rev and RRE sequences in HIV, which enable the efficient export of RNA transcripts of the integrated provirus from the nucleus to the cytoplasm of an infected target cell.

In the provirus, these genes are flanked at both ends by regions called long terminal repeats (LTRs). The LTRs are responsible for proviral integration and transcription. LTRs also serve as enhancer-promoter sequences and can control the expression of the viral genes.

The LTRs themselves are identical sequences that can be divided into three elements: U3, R and U5. U3 is derived from the sequence unique to the 3′ end of the RNA. R is derived from a sequence repeated at both ends of the RNA. U5 is derived from the sequence unique to the 5′ end of the RNA. The sizes of the three elements can vary considerably among different retroviruses.

In a defective retroviral vector genome gag, pol and env may be absent or not functional.

In a typical retroviral vector, at least part of one or more protein coding regions essential for replication may be removed from the virus. This makes the viral vector replication-defective. Portions of the viral genome may also be replaced by a library encoding candidate modulating moieties operably linked to a regulatory control region and a reporter moiety in the vector genome in order to generate a vector comprising candidate modulating moieties which is capable of transducing a target host cell and/or integrating its genome into a host genome.

Lentivirus vectors are part of the larger group of retroviral vectors. A detailed list of lentiviruses may be found in Coffin, J. M. et al. (1997) Retroviruses, Cold Spring Harbour Laboratory Press, 758-63. In brief, lentiviruses can be divided into primate and non-primate groups. Examples of primate lentiviruses include but are not limited to human immunodeficiency virus (HIV), the causative agent of human acquired immunodeficiency syndrome (AIDS); and simian immunodeficiency virus (SIV). Examples of non-primate lentiviruses include the prototype “slow virus” visna/maedi virus (VMV), as well as the related caprine arthritis-encephalitis virus (CAEV), equine infectious anaemia virus (EIAV), and the more recently described feline immunodeficiency virus (FIV) and bovine immunodeficiency virus (BIV).

The lentivirus family differs from retroviruses in that lentiviruses have the capability to infect both dividing and non-dividing cells (Lewis, P et al. (1992) EMBO J. 11: 3053-8; Lewis, P. F. et al. (1994) J. Virol. 68: 510-6). In contrast, other retroviruses, such as MLV, are unable to infect non-dividing or slowly dividing cells such as those that make up, for example, muscle, brain, lung and liver tissue.

A lentiviral vector, as used herein, is a vector which comprises at least one component part derivable from a lentivirus. Preferably, that component part is involved in the biological mechanisms by which the vector infects cells, expresses genes or is replicated.

The lentiviral vector may be a “primate” vector. The lentiviral vector may be a “non-primate” vector (i.e. derived from a virus which does not primarily infect primates, especially humans). Examples of non-primate lentiviruses may be any member of the family of lentiviridae which does not naturally infect a primate.

As examples of lentivirus-based vectors, HIV-1- and HIV-2-based vectors are described below.

The HIV-1 vector contains cis-acting elements that are also found in simple retroviruses. It has been shown that sequences that extend into the gag open reading frame are important for packaging of HIV-1. Therefore, HIV-1 vectors often contain the relevant portion of gag in which the translational initiation codon has been mutated. In addition, most HIV-1 vectors also contain a portion of the env gene that includes the RRE. Rev binds to RRE, which permits the transport of full-length or singly spliced mRNAs from the nucleus to the cytoplasm. In the absence of Rev and/or RRE, full-length HIV-1 RNAs accumulate in the nucleus. Alternatively, a constitutive transport element from certain simple retroviruses such as Mason-Pfizer monkey virus can be used to relieve the requirement for Rev and RRE. Efficient transcription from the HIV-1 LTR promoter requires the viral protein Tat.

Most HIV-2-based vectors are structurally very similar to HIV-1 vectors. Similar to HIV-1-based vectors, HIV-2 vectors also require RRE for efficient transport of the full-length or singly spliced viral RNAs.

In one system, the vector and helper constructs are from two different viruses, and the reduced nucleotide homology may decrease the probability of recombination. In addition to vectors based on the primate lentiviruses, vectors based on FIV have also been developed as an alternative to vectors derived from the pathogenic HIV-1 genome. The structures of these vectors are also similar to the HIV-1 based vectors.

Preferably, the viral vector used in the present invention has a minimal viral genome.

By “minimal viral genome” it is to be understood that the viral vector has been manipulated so as to remove the non-essential elements and to retain the essential elements in order to provide the required functionality to infect, transduce and deliver a nucleotide sequence of interest to a target host cell. Further details of this strategy can be found in WO 1998/017815.

Preferably, the plasmid vector used to produce the viral genome within a host cell/packaging cell will have sufficient lentiviral genetic information to allow packaging of an RNA genome, in the presence of packaging components, into a viral particle which is capable of infecting a target cell, but is incapable of independent replication to produce infectious viral particles within the final target cell. Preferably, the vector lacks a functional gag-pol and/or env gene and/or other genes essential for replication.

However, the plasmid vector used to produce the viral genome within a host cell/packaging cell will also include transcriptional regulatory control sequences operably linked to the lentiviral genome to direct transcription of the genome in a host cell/packaging cell. These regulatory sequences may be the natural sequences associated with the transcribed viral sequence (i.e. the 5′ U3 region), or they may be a heterologous promoter, such as another viral promoter (e.g. the CMV promoter).

The vectors may be self-inactivating (SIN) vectors in which the viral enhancer and promoter sequences have been deleted. SIN vectors can be generated and transduce non-dividing cells in vivo with an efficacy similar to that of wild-type vectors. The transcriptional inactivation of the long terminal repeat (LTR) in the SIN provirus should prevent mobilisation by replication-competent virus. This should also enable the regulated expression of genes from internal promoters by eliminating any cis-acting effects of the LTR.

The vectors may be integration-defective. Integration defective lentiviral vectors (IDLVs) can be produced, for example, either by packaging the vector with catalytically inactive integrase (such as an HIV integrase bearing the D64V mutation in the catalytic site; Naldini, L. et al. (1996) Science 272: 263-7; Naldini, L. et al. (1996) Proc. Natl. Acad. Sci. USA 93: 11382-8; Leavitt, A. D. et al. (1996) J. Virol. 70: 721-8) or by modifying or deleting essential att sequences from the vector LTR (Nightingale, S. J. et al. (2006) Mol. Ther. 13: 1121-32), or by a combination of the above.

Pharmaceutical Composition

In some embodiments, the population of genome edited cells produced or prepared according to a method of the invention may be formulated for administration to subjects with a pharmaceutically acceptable carrier, diluent or excipient. Suitable carriers and diluents include isotonic saline solutions, for example phosphate-buffered saline, and potentially contain human serum albumin.

Handling of the cell therapy product is preferably performed in compliance with FACT-JACIE International Standards for cellular therapy.

Therapies and Cell Transplantation

The present invention provides a population of genome edited cells, produced or prepared according to a method of the invention for use in therapy. In some embodiments, a population of genome edited cells is used in gene therapy. In some embodiments, a population of genome edited cells may be used for hematopoietic stem cell transplantations. In some embodiments, a population of genome edited cells may be used for cancer treatments (such as for the treatment of myeloma or leukaemia). In some embodiments, a population of genome edited cells may be used for tissue repair such as the repair of tissues in skin diseases (e.g. dermis disease and epidermolysis bullosa) or retinal disease (e.g. retinitis pigmentosa and Leber's congenital amaurosis).

The term “gene therapy” as used herein refers to modifications to the genome of a cell that restore function of a defective essential gene or abolish function of a disease gene. Gene therapy may be lentiviral based or AAV based.

The use may be as part of a cell transplantation procedure, for example a haematopoietic stem cell transplantation procedure.

Haematopoietic stem cell transplantation (HSCT) is the transplantation of blood stem cells derived from the bone marrow (in this case known as bone marrow transplantation) or blood. Stem cell transplantation is a medical procedure in the fields of haematology and oncology, most often performed for people with diseases of the blood or bone marrow, or certain types of cancer.

Many recipients of HSCTs are multiple myeloma or leukaemia patients who would not benefit from prolonged treatment with, or are already resistant to, chemotherapy. Candidates for HSCTs include paediatric cases where the patient has an inborn defect such as severe combined immunodeficiency (SCID) or congenital neutropenia with defective stem cells, and also children or adults with aplastic anaemia who have lost their stem cells after birth. Other conditions treated with stem cell transplants include sickle-cell disease, myelodysplastic syndrome, neuroblastoma, lymphoma, Ewing's Sarcoma, Desmoplastic small round cell tumour and Hodgkin's disease. More recently non-myeloablative, or so-called “mini transplant”, procedures have been developed that require smaller doses of preparative chemotherapy and radiation. This has allowed HSCT to be conducted in the elderly and other patients who would otherwise be considered too weak to withstand a conventional treatment regimen.

In some embodiments, a population of genome edited cells prepared according to a method of the invention is administered as part of an autologous cell transplant procedure.

In other embodiments, a population of genome edited cells prepared according to a method of the invention is administered as part of an allogeneic cell transplant procedure.

The term “autologous stem cell transplant procedure” as used herein refers to a procedure in which the starting population of cells (which are then genome edited according to a method of the invention) is obtained from the same subject as that to which the population of genome edited cells is administered. Autologous transplant procedures are advantageous as they avoid problems associated with immunological incompatibility and are available to subjects irrespective of the availability of a genetically matched donor.

The term “allogeneic stem cell transplant procedure” as used herein refers to a procedure in which the starting population of cells (which are then genome edited according to a method of the invention) is obtained from a different subject as that to which the population of genome edited cells is administered. Preferably, the donor will be genetically matched to the subject to which the cells are administered to minimise the risk of immunological incompatibility.

Suitable doses of a population of genome edited cells are such as to be therapeutically and/or prophylactically effective. The dose to be administered may depend on the subject and condition to be treated, and may be readily determined by a skilled person.

Haematopoietic progenitor cells provide short term engraftment. Accordingly, gene therapy by administering transduced haematopoietic progenitor cells would provide a non-permanent effect in the subject. For example, the effect may be limited to 1-6 months following administration of the transduced haematopoietic progenitor cells.

Such haematopoietic progenitor cell gene therapy may be suited to treatment of acquired disorders, for example cancer, where time-limited expression of a (potentially toxic) anti-cancer nucleotide of interest may be sufficient to eradicate the disease.

The population of genome edited cells may be useful in the treatment of the disorders listed in WO 1998/005635. For ease of reference, part of that list is now provided: cancer, inflammation or inflammatory disease, dermatological disorders, fever, cardiovascular effects, haemorrhage, coagulation and acute phase response, cachexia, anorexia, acute infection, HIV infection, shock states, graft-versus-host reactions, autoimmune disease, reperfusion injury, meningitis, migraine and aspirin-dependent anti-thrombosis; tumour growth, invasion and spread, angiogenesis, metastases, malignant, ascites and malignant pleural effusion; cerebral ischaemia, ischaemic heart disease, osteoarthritis, rheumatoid arthritis, osteoporosis, asthma, multiple sclerosis, neurodegeneration, Alzheimer's disease, atherosclerosis, stroke, vasculitis, Crohn's disease and ulcerative colitis; periodontitis, gingivitis; psoriasis, atopic dermatitis, chronic ulcers, epidermolysis bullosa; corneal ulceration, retinopathy and surgical wound healing; rhinitis, allergic conjunctivitis, eczema, anaphylaxis; restenosis, congestive heart failure, endometriosis, atherosclerosis or endosclerosis.

In addition, or in the alternative, the population of genome edited cells may be useful in the treatment of the disorders listed in WO 1998/007859. For ease of reference, part of that list is now provided: cytokine and cell proliferation/differentiation activity; immunosuppressant or immunostimulant activity (e.g. for treating immune deficiency, including infection with human immune deficiency virus; regulation of lymphocyte growth; treating cancer and many autoimmune diseases, and to prevent transplant rejection or induce tumour immunity); regulation of haematopoiesis, e.g. treatment of myeloid or lymphoid diseases; promoting growth of bone, cartilage, tendon, ligament and nerve tissue, e.g. for healing wounds, treatment of burns, ulcers and periodontal disease and neurodegeneration; inhibition or activation of follicle-stimulating hormone (modulation of fertility); chemotactic/chemokinetic activity (e.g. for mobilising specific cell types to sites of injury or infection); haemostatic and thrombolytic activity (e.g. for treating haemophilia and stroke); anti-inflammatory activity (for treating e.g. septic shock or Crohn's disease); as antimicrobials; modulators of e.g. metabolism or behaviour; as analgesics; treating specific deficiency disorders; in treatment of e.g. psoriasis, in human or veterinary medicine.

In addition, or in the alternative, the population of genome edited cells may be useful in the treatment of the disorders listed in WO 1998/009985. For ease of reference, part of that list is now provided: macrophage inhibitory and/or T cell inhibitory activity and thus, anti-inflammatory activity; anti-immune activity, i.e. inhibitory effects against a cellular and/or humoral immune response, including a response not associated with inflammation; inhibit the ability of macrophages and T cells to adhere to extracellular matrix components and fibronectin, as well as up-regulated of receptor expression in T cells; inhibit unwanted immune reaction and inflammation including arthritis, including rheumatoid arthritis, inflammation associated with hypersensitivity, allergic reactions, asthma, systemic lupus erythematosus, collagen diseases and other autoimmune diseases, inflammation associated with atherosclerosis, arteriosclerosis, atherosclerotic heart disease, reperfusion injury, cardiac arrest, myocardial infarction, vascular inflammatory disorders, respiratory distress syndrome or other cardiopulmonary diseases, inflammation associated with peptic ulcer, ulcerative colitis and other diseases of the gastrointestinal tract, hepatic fibrosis, liver cirrhosis or other hepatic diseases, thyroiditis or other glandular diseases, glomerulonephritis or other renal and urologic diseases, otitis or other oto-rhino-laryngological diseases, dermatitis or other dermal diseases, periodontal diseases or other dental diseases, orchitis or epididimo-orchitis, infertility, orchidal trauma or other immune-related testicular diseases, placental dysfunction, placental insufficiency, habitual abortion, eclampsia, pre-eclampsia and other immune and/or inflammatory-related gynaecological diseases, posterior uveitis, intermediate uveitis, anterior uveitis, conjunctivitis, chorioretinitis, uveoretinitis, optic neuritis, intraocular inflammation, e.g. retinitis or cystoid macular oedema, sympathetic ophthalmia, scleritis, retinitis pigmentosa, immune and inflammatory components of degenerative fondus disease, inflammatory components of ocular trauma, ocular inflammation caused by infection, proliferative vitreo-retinopathies, acute ischaemic optic neuropathy, excessive scarring, e.g. following glaucoma filtration operation, immune and/or inflammation reaction against ocular implants and other immune and inflammatory-related ophthalmic diseases, inflammation associated with autoimmune diseases or conditions or disorders where, both in the central nervous system (CNS) or in any other organ, immune and/or inflammation suppression would be beneficial, Parkinson's disease, complication and/or side effects from treatment of Parkinson's disease, AIDS-related dementia complex HIV-related encephalopathy, Devic's disease, Sydenham chorea, Alzheimer's disease and other degenerative diseases, conditions or disorders of the CNS, inflammatory components of stokes, post-polio syndrome, immune and inflammatory components of psychiatric disorders, myelitis, encephalitis, subacute sclerosing pan-encephalitis, encephalomyelitis, acute neuropathy, subacute neuropathy, chronic neuropathy, Guillaim-Barre syndrome, Sydenham chora, myasthenia gravis, pseudo-tumour cerebri, Down's Syndrome, Huntington's disease, amyotrophic lateral sclerosis, inflammatory components of CNS compression or CNS trauma or infections of the CNS, inflammatory components of muscular atrophies and dystrophies, and immune and inflammatory related diseases, conditions or disorders of the central and peripheral nervous systems, post-traumatic inflammation, septic shock, infectious diseases, inflammatory complications or side effects of surgery, bone marrow transplantation or other transplantation complications and/or side effects, inflammatory and/or immune complications and side effects of gene therapy, e.g. due to infection with a viral carrier, or inflammation associated with AIDS, to suppress or inhibit a humoral and/or cellular immune response, to treat or ameliorate monocyte or leukocyte proliferative diseases, e.g. leukaemia, by reducing the amount of monocytes or lymphocytes, for the prevention and/or treatment of graft rejection in cases of transplantation of natural or artificial cells, tissue and organs such as cornea, bone marrow, organs, lenses, pacemakers, natural or artificial skin tissue.

In addition, or in the alternative, the population of genome edited cells may be useful in the treatment of β-thalassemia, chronic granulomatous disease, metachromatic leukodystrophy, mucopolysaccharidoses disorders and other lysosomal storage disorders.

In some embodiments, the population of genome edited cells is useful in the treatment of Severe Combined Immunodeficiency (SCID), such as X-linked SCID.

In some embodiments, the population of genome edited cells is useful in the treatment of a skin disease, such epidermolysis bullosa.

In some embodiments, the population of genome edited cells is useful in the treatment of a monogeneic disorder, such as epidermolysis bullosa and/or retinitis pigmentosa and/or Hyper-IgM (HIGM) syndrome.

In some embodiments, the population of genome edited cells is useful in the treatment of a retinal disease such as retinitis pigmentosa and/or Leber's congenital amaurosis).

In some embodiments, the population of genome edited cells is useful in the treatment of epidermolysis bullosa.

In some embodiments, the population of genome edited cells is useful in the treatment of retinitis pigmentosa.

In some embodiments, the population of genome edited cells is useful in the treatment of Leber's congenital amaurosis.

In some embodiments, the population of genome edited cells is useful in the treatment of Hyper-IgM (HIGM) syndrome.

Kit

In one aspect, there is provided a kit comprising a first component as defined herein, a second component as defined herein and a third component as defined herein and, optionally, a cell population.

The components and, optionally, the cell population may be provided in suitable containers.

The kit may also include instructions for use.

Method of Treatment

It is to be appreciated that all references herein to treatment include curative, palliative and prophylactic treatment; although in the context of the invention references to preventing are more commonly associated with prophylactic treatment. In some embodiments, the treatment of mammals, particularly humans, is preferred. Both human and veterinary treatments are within the scope of the invention.

The skilled person will understand that they can combine all features of the invention disclosed herein without departing from the scope of the invention as disclosed.

Administration

Although the population of genome edited cells for use in the invention can be administered alone, they will generally be administered in admixture with a pharmaceutical carrier, excipient or diluent, particularly for human therapy.

Dosage

The skilled person can readily determine an appropriate dose of the population of genome edited cells to administer to a subject without undue experimentation. Typically, a physician will determine the actual dosage which will be most suitable for an individual patient and it will depend on a variety of factors including the activity of the specific agent employed, the metabolic stability and length of action of that agent, the age, body weight, general health, sex, diet, mode and time of administration, rate of excretion, drug combination, the severity of the particular condition, and the individual undergoing therapy. There can of course be individual instances where higher or lower dosage ranges are merited, and such are within the scope of the invention.

Subject

A “subject” refers to either a human or non-human animal.

Examples of non-human animals include vertebrates, for example mammals, such as non-human primates (particularly higher primates), dogs, rodents (e.g. mice, rats or guinea pigs), pigs and cats. The non-human animal may be a companion animal.

Preferably, the subject is a human.

Preferred features and embodiments of the invention will now be described by way of non-limiting examples.

The practice of the present invention will employ, unless otherwise indicated, conventional techniques of chemistry, biochemistry, molecular biology, microbiology and immunology, which are within the capabilities of a person of ordinary skill in the art. Such techniques are explained in the literature. See, for example, Sambrook, J., Fritsch, E. F. and Maniatis, T. (1989) Molecular Cloning: A Laboratory Manual, 2nd Edition, Cold Spring Harbor Laboratory Press; Ausubel, F. M. et al. (1995 and periodic supplements) Current Protocols in Molecular Biology, Ch. 9, 13 and 16, John Wiley & Sons; Roe, B., Crabtree, J. and Kahn, A. (1996) DNA Isolation and Sequencing: Essential Techniques, John Wiley & Sons; Polak, J. M. and McGee, J. O'D. (1990) In Situ Hybridization: Principles and Practice, Oxford University Press; Gait, M. J. (1984) Oligonucleotide Synthesis: A Practical Approach, IRL Press; and Lilley, D. M. and Dahlberg, J. E. (1992) Methods in Enzymology: DNA Structures Part A: Synthesis and Physical Analysis of DNA, Academic Press. Each of these general texts is herein incorporated by reference.

### EXAMPLES

### Example 1

Materials and Methods

Vectors and Nucleases

AAV6 donor templates for HDR were generated from a construct containing AAV2 inverted terminal repeats, produced by triple-transfection method and purified by ultracentrifugation on a cesium chloride gradient as previously described (1). Design of AAV-6 donor templates with homologies for AAVS1 locus (encoding for minCMV.GFP or SK.GFP reporter cassette) or targeting the intron 1 of IL2RG (encoding for IL2RG corrective cDNA followed by either a PGK.GFP reporter cassette or 2A.NGFR selector cassette) was previously reported (2). Sequences of the gRNAs for gene targeting were designed using an online CRISPR design tool (4) and selected for predicted specificity score and on target activity. Genomic sequences recognized by the gRNAs are indicated below.

TABLE A
genomic sequences recognized by the gRNA.

| Guide ID | Genomic sequence (5′-3′) |
|---|---|
| AAVS1 gRNA | GTCACCAATCCTGTCCCTAGTGG |
| IL2RG gRNA | ACTGGCCATTACAATCATGTGGG |

(SEQ ID NO: 71 and SEQ ID NO: 72, respectively.)

Ribonucleoproteins (RNPs) were assembled by incubating at 1:1.5 molar ratio s.p.Cas9 protein (Aldevron) with synthetic cr:tracrRNA (Integrated DNA Technologies) for 10 minutes at 25° C. Electroporation enhancer (Integrated DNA Technologies) was added prior to electroporation according to manufacturer's instructions.

gRNA sequences for transactivation platforms are reported in Table 1. Sp-dCas9-VPR plasmid has been bought from Addgene (#63789) and Sp-dCas9-VP160 has been cloned from plasmid #47107 (Addgene). TALE has been cloned by Golden Gate strategy in pUC plasmid and VPR domain from plasmid #63789 fused at the C-terminus. TALE binding sites are listed in Table 2. For mRNA in vitro transcription TALE#7- or TALE#3-VPR or VP160 construct were cloned in a pVax plasmid containing a T7 promoter, β-globin 3′UTR and 64bp-polyA.

TABLE 1

| Guide | | | Distance | Orien- |
|---|---|---|---|---|
| ID | Target sequence | Score | (bp) | tation |
| #1 | TGGGGGTTAGACCCAATATCAGG | 88 | 80 | AS |
| #2 | CCTTCCTAGTCTCCTGATATTGG | 70 | 68 | S |
| #3 | CCAATATCAGGAGACTAGGAAGG | 71 | 68 | AS |
| #4 | AGATAAGGAATCTGCCTAACAGG | 76 | 105 | AS |
| #5 | TGTTAGGCAGATTCCTTATCTGG | 73 | 107 | S |
| #6 | TGGGGGTGTGTCACCAGATAAGG | 72 | 121 | AS |
| #7 | TCTCCTTGCCAGAACCTCTAAGG | 70 | 161 | S |
| #8 | CCTCTAAGGTTTGCTTACGATGG | 92 | 174 | S |
| #9 | AAACCTTAGAGGTTCTGGCAAGG | 72 | 164 | AS |
| #10 | TAAGCAAACCTTAGAGGTTCTGG | 72 | 179 | AS |
| #11 | GTGACCTGCCCGGTTCTCAGTGG | 70 | 259 | S |
| #12 | GGGGGGATGCGTGACCTGCCCGG | 68 | 249 | S |
| #13 | GTTCTGGGAGAGGGTAGCGCAGG | 80 | 280 | AS |
| #14 | GCTGCTCTGACGCGGCCGTCTGG | 92 | 312 | S |
| #15 | GAACCTGAGCTGCTCTGACGCGG | 73 | 303 | S |
| #16 | CGGCCGCGTCAGAGCAGCTCAGG | 75 | 307 | AS |
| #17 | CTGGTGCGTTTCACTGATCCTGG | 83 | 331 | S |
| #18 | GCTTCCTTACACTTCCCAAGAGG | 60 | 361 | S |
| #19 | GATCAGTGAAACGCACCAGACGG | 83 | 327 | AS |
| #20 | TTGGTCCTGAGTTCTAACTTTGG | 70 | 416 | S |
| #21 | TCCCAAGAGGAGAAGCAGTTTGG | 56 | 374 | AS |
| #22 | CGGAGGAACAATATAAATTGGGG | 67 | 458 | AS |
| #23 | TCACAGGTAAAACTGACGCACGG | 77 | 475 | AS |
| #24 | GCCAGTAGCCAGCCCCGTCCTGG | 76 | 506 | S |
| #25 | GTAGCCAGCCCCGTCCTGGCAGG | 64 | 510 | S |
| #26 | GGCTACTGGCCTTATCTCACAGG | 83 | 493 | AS |
| #27 | CCTAGGTGTTCACCAGGTCGTGG | 79 | 580 | S |
| #28 | TTGTGAGAATGGTGCGTCCTAGG | 84 | 573 | S |
| #29 | GAGTAGAGGCGGCCACGACCTGG | 87 | 602 | AS |
| #30 | GTGCGTCCTAGGTGTTCACCAGG | 88 | 584 | S |
| #31 | AAAGAGTCCCCAGTGCTATCTGG | 77 | 654 | S |

#1-31 are SEQ ID Nos: 1-31.

TABLE 2
TALE DBDs

| TALE ID | Target sequence | Distance from TSS |
|---|---|---|
| TALE #1 | TCCATCGTAAGCAAACCTT | 324 |
| TALE #2 | TCCCACCCCCTGCCAAGCT | 367 |
| TALE #3 | TCCAAACTGCTTCTCCTCT | 523 |
| TALE #4 | TCCACACGGACACCCCCCT | 689 |
| TALE #5 | TCCACCATCTCATGCCCCT | 940 |
| TALE #6 | TCTAAGGTTTGCTTACGAT | 321 |
| TALE #7 | TCCTCTCTGGCTCCATCGT | 336 |
| TALE #8 | TGGTCCTGAGTTCTAACTT | 563 |
| TALE #9 | TCCTCCGTGCGTCAGTTTT | 617 |

TALE #1 - SEQ ID NO: 32
TALE #2 - SEQ ID NO: 33
TALE #3 - SEQ ID NO: 34
TALE #4 - SEQ ID NO: 35
TALE #5 - SEQ ID NO: 36
TALE #6 - SEQ ID NO: 37
TALE #7 - SEQ ID NO: 38
TALE #8 - SEQ ID NO: 39
TALE #9 - SEQ ID NO: 40

Gene Editing and Target Gene Transactivation in K562 Cell Line

The K562 cell line is a human immortalized myelogenous leukemia cell line; K562 cells are of the erythroleukemia type.

K562 cell lines were cultured in IMDM medium (GIBCO-BRL) supplemented with penicillin, streptomycin, glutamine and 10% FBS. For gene targeting experiments, 3×10⁵ cells were electroporated (SF Cell Line 4D- Nucleofector X Kit, program EW113; Lonza) with 50 μg/ml of plasmids encoding for donor DNA template and 2.5-1.25 μM of RNPs. Cells were then expanded to perform flow cytometry and/or molecular analyses. For transactivation experiments, targeted K562 cells were electroporated with 50 μg/ml of TALE-TA or dCas9-TA plasmids and 12.5 μg/ml of U6.sgRNA plasmids, where not differently indicated.

Gene Editing and Target Gene Transactivation in Human CD34+ Cells

CD34+ cells were either freshly purified from human cord blood (CB) after obtaining informed consent and upon approval by the Ospedale San Raffaele Bioethical Committee, or purchased frozen from Lonza. CD34+ cells were edited according to a previously optimized protocol (2). Briefly, 5×10⁵ CD34+ cells/ml were stimulated in serum-free StemSpan medium (StemCell Technologies) supplemented with penicillin, streptomycin, glutamine, 1 μM SR-1(Biovision), 50 μM UM171 (STEMCell Technologies), 10 μM PGE2 added only at the beginning of the culture (Cayman), and human early-acting cytokines (SCF 100 ng/ml, Flt3-L 100 ng/ml, TPO 20 ng/ml, and IL-6 20 ng/ml; all purchased from Peprotech). After 3 days of prestimulation, cells were washed with PBS and electroporated using P3 Primary Cell 4D-Nucleofector X Kit and program EO-100 (Lonza). Cells were electroporated with 2.5-1.25 μM of RNPs. Transduction with AAV6 was performed at a dose of 1-2×10⁴ vg/cell 15′ after electroporation. TALE-TA mRNA was utilized where indicated at a dose of 350 μg/ml. Transactivation efficiency was measured from cultured cells in vitro 1, 2 and 7 days after electroporation by flow cytometry measuring the percentage of cells expressing the GFP marker. Gene editing efficiency was measured by digital droplet PCR analysis designing primers and probe on the junction between the vector sequence and the targeted locus and on control sequences utilized as normalizer as previously described (2).

Beads-based selection of LNGFR+ cells has been performed with MACSelect LNGFR MicroBeads, accordingly to manufacturer's instructions.

CD34+ HSPC Xenotransplantation Studies in NSG Mice

NOD-SCID-IL2Rg^{-/-} (NSG) mice were purchased from The Jackson Laboratory and were maintained in specific-pathogen-free (SPF) conditions. The procedures involving animals were designed and performed with the approval of the Animal Care and Use Committee of the San Raffaele Hospital (IACUC #749) and communicated to the Ministry of Health and local authorities according to Italian law.

6×10⁵CD34+ cells treated for editing and transactivation at day 5 of culture were sorted and injected intravenously into NSG mice after sub-lethal irradiation (150-180 cGy). Sample size was determined by the total number of available treated cells. Mice were attributed to each experimental group randomly. Human CD45+ cell engraftment and the presence of gene-edited cells were monitored by serial collection of blood from the mouse tail and, at the end of the experiment (>20 weeks after transplantation), bone marrow (BM) and spleen were harvested and analyzed.

Molecular Analyses

For molecular analyses, genomic DNA was isolated with DNeasy Blood & Tissue Kit or QlAamp DNA Micro Kit (QIAGEN) according to the number of cell available. Nuclease activity (IL2RG intron 1, AAVS1) was measured by mismatch-sensitive endonuclease assay by PCR-based amplification of the targeted locus followed by digestion with T7 Endonuclease I (NEB) according to the manufacturer's instructions. Digested DNA fragments were resolved and quantified by capillary electrophoresis on LabChip GX Touch HT (Perkin Elmer) according to the manufacturer's instructions.

For digital droplet PCR analysis, 5-50 ng of genomic DNA were analyzed in duplicate using the QX200 Droplet Digital PCR System (Biorad) according to the manufacturer's instructions.

For HDR ddPCR, primers and probes were designed on the junction between the vector sequence and the targeted locus and on control sequences used for normalization (human TTC5 genes). Thermal conditions for annealing and extension were adjusted for each specific application as follows: AAVS1/ Intron 1 IL2RG HDR 3′ integration junction ddPCR: 55° C. for 30 sec, 72° C. for 2 min. Primers and probes for PCR and ddPCR amplifications are shown in Table C below.

TABLE C
Primers and probes for PCR and
ddPCR amplifications

| Description | Orientation | Sequence (5′-3′) |
|---|---|---|
| NHEJ AAVS1 | FW | CTTCAGGACAGCATGTTTGC |
| | | (SEQ ID NO: 77) |
| | RV | ACAGGAGGTGGGGGTTAGAC |
| | | (SEQ ID NO: 78) |
| NHEJ Intron 1 | FW | CACCCTCTGTAAAGCCCTGG |
| IL2RG | | (SEQ ID NO: 79) |
| | RV | AAGAAATCTAGATTGGGGAG |
| | | (SEQ ID NO: 80) |
| Intron 1 IL2RG | FW | CTAGATTGGGGAGAAAATGA |
| 3′ integration | | (SEQ ID NO: 81) |
| junction | | |
| ddPCR | | |
| | RV | GTGGGAAGGGGCCGTACAG |
| | | (SEQ ID NO: 82) |
| | Probe | GTAGCTCCTATGCTAGGCGTA |
| | (FAM) | GCC(SEQ ID NO: 83) |
| AAVS1 3′ | FW (SK) | CACCGTACACGCCTAAAGCTA |
| integration | | (SEQ ID NO: 84) |
| junction | | |
| ddPCR | | |
| | FW | CTTATATAGGCCTCCCACCGT |
| | (mCMV) | (SEQ ID NO: 85) |
| | RV | TCTTGGGAAGTGTAAGGAAG |
| | | (SEQ ID NO: 86) |
| | Probe | CCAGATAAGGAATCTGCCTA |
| | (FAM) | (SEQ ID NO: 87) |
| Human TTC5 | (HEX) | PrimePCR ddPCR Copy |
| ddPCR | | Number Assay: TTC5, |
| | | Human (Biorad) |

For gene expression analyses, total RNA was extracted using the RNeasy Plus Micro Kit (QIAGEN). cDNA was synthetized with SuperScript VILO IV cDNA Synthesis Kit (Invitrogen) and used for Q-PCR in a Viia7 Real-time PCR thermal cycler using TaqMan Gene Expression Assays (Applied Biosystems) mapping to IL2RG and HPRT as normalizer. The relative expression of each gene was first normalized to HPRT expression and then represented as fold change relative to the mock-treated sample.

Flow Cytometry

For immunophenotypic analyses (performed on FACSCanto II; BD Pharmingen), the antibodies listed below in Table B were used. Single stained and Fluorescence Minus One stained cells were used as controls. LIVE/DEAD Fixable Dead Cell Stain Kit (Thermo Fisher), 7-aminoactinomycin (Sigma Aldrich), were included in the sample preparation for flow cytometry according to the manufacturer's instructions to exclude dead cells from the analysis. Cell sorting was performed using MoFlo XDP Cell Sorter (Beckman Coulter) or FACSAria Fusion (BD Biosciences).

TABLE B
antibodies for immunophenotypic analyses.

| Anti-Human | | | |
|---|---|---|---|
| Antibody | Fluorochrome | Clone | Company |
| CD133/2 | PE | 293C3 | Miltenyi |
| | | | Biotec |
| CD34 | PB | AC 136 | Miltenyi |
| | | | Biotec |
| CD34 | PECy7 | 8G12 | BD |
| CD90 | APC | 5E10 | BD |
| CD45 | PB | HI30 | Biolegend |
| CD45 | APCH7 | HI30 | eBioscience |
| CD19 | PE | HIB19 | BD |
| CD19 | Pecy7 | HIB19 | Biolegend |
| CD3 | Pecy7 | HIT3a | Biolegend |
| CD3 | PE | SK7 | BD |
| CD13 | APC | WM15 | BD |
| CD33 | PeCy7 | P67.6 | BD |
| CD38 | APC | HB7 | BD |
| CD38 | Percp5.5 | HB7 | Biolegend |
| CD4 | PB | RPA-T4 | BD |
| CD271 | APC | C40-1477 | BD |
| CD271 | PE | ME20.4-1.H4 | BD |
| CD8 | APCH7 | SK1 | BD |

BD refers to BD Biosciences

Results

The Adeno-Associated Virus Integration Site 1 (AAVS1) locus is one of the most validated genomic harbors for targeted integration of cassettes expressing a gene of interest (GOI) (Lombardo et al., 2011). Therefore, we envisaged application of the SMArT strategy disclosed herein (FIG. 1A) in AAVS1 site. To this purpose, we performed targeted integration of a donor DNA containing ΔLNGFR cDNA under the control of a minimally expressing CMV-derived (minCMV) promoter in K562 erythroblastoid cell line. By performing single cell cloning from treated cells, we identified a clone with targeted integration of our construct (i.e. intact 5′ and 3′ vector-genome junctions) which was not expressing ΔLNGFR (clone H1) (FIG. 1B).

To set up our ETT platforms, we designed 29 sgRNAs targeting different sequences spanning the region upstream the minCMV promoter (Table 1) and we screened them for cleavage activity in non-saturating conditions by delivering the U6.sgRNA plasmid in the clone H1. We observed that almost all the sgRNAs cut the target sequence with efficiencies above 60% (FIG. 1C).

The most widely exploited transactivating domains for target gene activation are multimeric VP16-derived proteins (such as VP64, VP128, VP160) and the tripartite transactivator VPR (VP64-p65-Rta) (Chavez et al., 2015). We fused a catalytically inactive S.p. Cas9 protein (dCas9) (harboring D10A and H840A mutations) (Jinek et al., Science 2012; 377(6096): 816-821; PMID: 2275249) with either VP160 or VPR domain and we tested the ETTs in combination with some of the previously screened sgRNAs. While ETT alone did not spuriously transactivate ΔLNGFR expression, co-delivery of the sgRNA with the ETT expressing plasmid resulted in efficient and transient transactivation with 3 out of 8 guides tested. By delivering the best performing sgRNA, we achieved up to 85% of ΔLNGFR+ cells at 48 hours after electroporation and 12- and 20-fold of ΔLNGFR cell-surface expression (measured as relative fluorescence intensity [RFI] over untreated control) with dCas9-VP160 or −VPR, respectively (FIGS. 1D and 1E). Remarkably, we observed a strong synergistic effect when we delivered equal amounts of two of the less potent sgRNAs targeting different sequences upstream from the minCMV promoter. These data are in line with the finding that VPR is more potent compared with VP16-derived transactivators and combination of multiple guides to drive the same ETT strongly enhance target gene activation (Chavez et al., 2016). Moreover, our data confirm that gene transactivation is more potent when DNA target sequences are close to the transcriptional start site (TSS) and, in particular, between 100 to 250 bp (Gilbert et al., 2014; Konermann et al., 2015).

To assess whether our platform is also portable to other technologies, we fused Transcriptional Activator-Like Effector (TALE) DBDs, which recognize 9 different target sequences upstream from the minCMV promoter (Table 2), with VP160 transactivating domains and we delivered TALE-VP160 expressing plasmids in the clone H1. We found that 3 out of 9 ETTs efficiently transactivated ΔLNGFR expression and, interestingly, the most potent ETT (TALE #7) had the same binding specificity of the best performing sgRNA (FIG. 1F). Moreover, more efficient transactivation and reduced toxicity were achieved when clone H1 was electroporated with mRNA encoding for TALE7 ETT, with a peak of NGFR expression between 12 and 48 hours after treatment (FIG. 1G).

In principle, enrichment of gene-modified cells might be achieved by co-delivering the ETT together with the editing machinery (“early selection”) or by postponing ETT delivery some days after gene targeting procedure (“late selection”). In the first option, ETT binding sites must necessarily map on the genome outside the HA in order to avoid episomal donor transactivation. Therefore, we tailored the design of our vector (containing the selector cassette and the GOI cassette) to shorten the left HA and we observed only minimal impact on gene targeting efficiency when using one HA smaller than 300 bp in AAVS1 locus (FIG. 2A). To get the best compromise between efficient gene targeting and transient transactivation, we utilized HDR donor constructs with about 150 bp left HA, thus avoiding the presence of TALE #7 and sgRNA #10 binding site in the donor template. Since a relevant fraction of cells harboring minCMV-ΔLNGFR targeted integration in AAVS1 locus in K562 cell line presents ΔLNGFR on cell surface even in absence of any transactivation, we improved HDR donor design by substituting this promoter (minCMV) with its improved synthetic version “T6-SK” (referred to as SK in FIG. 2B), which has been reported to reduce promoter leakage without affecting inducible expression capacity in a Tet-ON system configuration (Loew et al., BMC Biotechnol. 2010). Upon targeted integration, we consistently observed about 2-fold less (but not abolished) steady-state expression of SK-ΔLNGFR cassette as compared with minCMV-expressing one, with the vast majority of the cells basally expressing ΔLNGFR, as confirmed by ddPCR-based quantification of gene targeting efficiency. On the contrary, when performing gene targeting of a promoter-less ΔLNGFR construct only a small fraction of cells presented ΔLNGFR on their surface (FIG. 2B). To validate our SMArT strategy in a “late selection” setting, we electroporated CRISPR/dCas9-based ETTs two weeks after gene targeting procedure and obtained efficient and transient ΔLNGFR overexpression (FIG. 2C). Similarly, to validate the SMArT strategy in an “early selection” setting, we co-electroporated in K562 the CRISPR/dCas9-based ETTs together with the gene editing reagents for AAVS1 targeting. By performing single cell sorting on ΔLNGFR^{high} cells at 24 hours after ETT delivery we observed that almost all the clones harbored molecular evidence of targeted integration at the AAVS1 target site (at least one intact donor-genome junction), thus providing the proof-of-principle that selection by SMArT strategy allows to enrich for on-target edited cells (FIG. 2D).

Hematopoietic stem cell (HSC) gene therapy has recently shown clear benefit for patients affected by either hematological or non-hematological inherited disorders. Targeted genome editing in HSCs would allow to improve the safety of gene therapy approaches by avoiding insertional mutagenesis and providing physiological regulation of the corrective transgene. However, the broader applicability of gene targeting-based approaches is limited by the efficiency of HDR-mediated targeted integration, especially in long-term repopulating progenitors. To investigate whether the SMArT strategy could be coupled with gene editing protocol in hematopoietic stem and progenitor cells (HSPCs), we performed gene targeting experiments in cord blood (CB)-derived CD34+ cells and we delivered by a single electroporation both AAVS1-specific nucleases (as RNP) and TALE#7 ETTs (as mRNA) (FIG. 3A). As expected, we observed lower basal reporter gene (GFP) expression with both minCMV and T6-SK promoters in HSPCs as compared with K562 cell line. We detected low but appreciable transactivation of GFP expression with both constructs in presence of ETTs, which was more pronounced with TALE#7-VPR, albeit more efficient in committed progenitors (FIG. 3B). We observed that a high fraction of edited HSPCs overexpressed GFP upon transactivation (as suggested by molecular quantification of HDR-mediated integration) (FIG. 3C). To validate our “late selection” strategy, we electroporated TALE#7-VPR mRNA 3-4 days after gene editing procedure. Here, we observed more potent transactivation capacity (both in terms of percentage of GFP+ cells and GFP MFI) as compared with the early selection protocol, although still more efficient in the more committed progenitors (FIG. 3D). These results provide the proof-of-principle that SMArT strategy allows to select AAVS1-edited HSPCs upon transient transactivation of a selector protein.

To further increase the expression of the selector gene, the SMArT strategy could be applied in presence of translational activator, such as a modular RNA activator containing the aptamer for eukaryotic initiation factor 4G (eIF4G) that activated target mRNA translation in a 5′-UTR independent manner (Liu et al., 2018). In more detail, one or multiple binding sites for the RNA aptamer will be inserted in the corrective construct just downstream of the TSS in order to boost mRNA translation. Another possible approach to increase the expression of the selector exploits the enhancer activity of the promoter (such as EF1alpha promoter) used to drive the expression of the therapeutic gene of interest. Without wishing to be bound by theory, the presence of a strong enhancer/promoter region nearby the minimal promoter might improve transactivation efficiency on the selector gene by recruiting more transcription core factors on-site.

In parallel, we developed a similar strategy, named Selection by Means of Artificial Transactivation of Endogenous Receptors (SMArTER), which is based on transient transactivation of endogenous receptors, that can be suitable for the enrichment of cells that underwent site-specific correction of a gene whose product does not provide selective growth advantage or may not be amenable for selection (e.g. intracellular protein, not suitable for antibody-mediated recognition, etc . . . ). In these settings, expression of the selector protein depends on the transcriptional activity on the corrected gene that will be transiently transactivated by means of ETTs, thus allowing selection of the edited cells also when the corrected gene is not sufficiently expressed in target cells (FIG. 4A). As proof of concept, we applied this strategy in the context of gene correction of Interleukin 2 Receptor Common γ-chain (IL2RG) gene, the gene mutated in the X-linked Severe Combined Immunodeficiency (SCID-X1). By integrating a corrective cDNA in the intron 1 of this gene in male human HSPCs, we have recently proved that IL2RG-edited HSCs are capable of long-term persistence in immunodeficient NSG mice and results in functional multilineage reconstitution (Schiroli et al., 2017). In SCID-X1 disease, even a few corrected progenitors could rescue the disease phenotype. However, the input of functional progenitors inversely correlates with the risk of thymic lymphoma due to the absence of competition for thymic repopulation from BM-derived CLPs (Ginn et al., 2017; Martins et al., 2014; Schiroli et al., 2017). Despite gene correction levels in IL2RG locus in human HSPCs overpass the threshold for safe and effective treatment of SCID-X1 disease, enrichment of IL2RG-edited HSPCs before in vivo administration might allow to further reduce any theoretical risk of malignancies and minimize administration of HSPCs harboring off-target integrations. Moreover, ex vivo selection of IL2RG-edited HSPCs could work as a model portable to site-specific correction of other genes, such as gp91phox, HBB, RAG1, CD40LG, TRAC, TRBC, STAT, PRF1 or genes encoding for a protein expressed in the skin such as collagen, keratin, laminin, desmocolin, desmoplachine, desmoglein, placoglobin, placophylline, integrin or other proteins that are involved in desmosomes and hemidesmosomes.

We first designed three TALE-VP160 that bind different regions of IL2RG promoter close to TATA box consensus. To test whether these TALE-based ETTs were capable of boosting gamma-chain expression, we separately electroporated TALE-VP160-expressing plasmids in K562 cell line, which basally expresses IL2RG mRNA to detectable levels (differently from HEK293T cell line). We found that only TALE#3-VP160 (referred to as T3 in the Figure) was able to induce 11-fold IL2RG overexpression, thus achieving IL2RG mRNA levels comparable to those basally measured in a male B-lymphoblastoid cell line (JY) (FIG. 4B). Therefore, we selected TALE#3 as candidate for IL2RG transactivation.

In order to assess the feasibility of our selection strategy in human HSPCs, we generated a reporter for checking IL2RG promoter activity by targeting a splicing acceptor (SA).T2A.GFP cassette within the intron 1 of the gene and we co-delivered TALE#3-VP160 or TALE#3-VPR mRNA. We found no impact of transactivation activity on gene targeting efficiency but we observed a significant improvement in GFP MFI in all HSPC subpopulations in presence of TALE#3-VP160 and, especially, of TALE#3-VPR (FIGS. 4C and 4D). To reconstitute IL2RG gene expression and develop a potentially clinically compliant selection strategy for this gene, we substituted the T2A.GFP cassette with a larger construct containing the corrective codon-optimized IL2RG cDNA followed by the T2A.ΔLNGFR reporter gene. To functionally validate the IL2RGrec.2A.NGFR construct on a human cell type that strictly depends on gamma-chain expression for proliferation, we compared it with the previously published optimized donor DNA for IL2RG gene correction (IL2RGrec PGK-GFP) in primary male T lymphocytes (Schiroli et al., 2017). Despite showing about 20% lower IL2RG expression on membrane surface of edited cells compared to not edited counterpart, the subset of cells edited with the 2A.NGFR construct grew similarly in culture as the control edited cells. Moreover, HDR-modified cells were not counter-selected in culture over time, thus indicating the functionality of the 2A.NGFR corrective construct (FIG. 5A). We then performed selection experiment in CB-derived CD34+ cells by using IL2RGrec.2A.NGFR as donor DNA for HDR and TALE#3-VPR s for transient IL2RG promoter transactivation. Differently from 2A.GFP targeted integration, ΔLNGFR was not basally expressed on the surface of edited cells (and in particular in more primitive HSPCs) in absence of any transactivation, probably due to low sensitivity of anti-ΔLNGFR antibody. Therefore, TALE#3VPR-mediated transactivation was required to potentially select edited cells (FIGS. 5B and C). To further assess whether enrichment of edited HSPCs was feasible upon transient overexpression of ΔLNGFR reporter gene, we performed either FACS or magnetic beads selection of reporter-expressing cells 36 hours upon electroporation. Both selection methods resulted in about 75% ΔLNGFR+ cells in the positively selected cell subset, with up to 90% of HDR editing measured by molecular analysis in the ΔLNGFR+ FACS-sorted fraction. We also measured consistent levels of HDR in the ΔLNGFR− fraction, thus suggesting that our selection process has an approximate yield of 50%. Moreover, we observed only a minor difference in subpopulation composition between ΔLNGFR+ sorted and unsorted HSPCs, showing that our procedure is not strongly biased towards enrichment of more committed progenitors (FIGS. 5C and D).

The SMArTER strategy could be further refined by adding to the selector gene a ligand-regulatable destabilizing domain, such as those based on the FKBP domain (Banaszynski et al., 2006) or from the from the Human Estrogen Receptor (Miyazaki et al., 2012), with the aim to avoid expression of the selector in the differentiated cells that will physiologically express the edited gene. If the SMArTER strategy is applied to correct inherited mutations that generates an absence of expression of the affected gene, and if the affected gene is a surface exposed protein, such in the case of IL2RG in SCID-X1, the SMArTER strategy could be further optimized by exploiting the corrected gene itself as selector marker. In this case, binding of ETTs on the endogenous promoter of the corrected gene will result in the overexpression of the cell surface protein, which can be used as surface marker for FACS- or magnetic beads-mediated enrichment of corrected cells.

### Example 2

Methods

Vectors and Nucleases

AAV6 donor templates for HDR were generated from a construct containing AAV2 inverted terminal repeats, produced by triple-transfection method and purified by ultracentrifugation on a cesium chloride gradient as previously described (Wang et al., 2015). Design of AAV6 donor templates is detailed in the Supplementary Material Section.

Sequences of the gRNAs were designed using an online CRISPR design tool (Hsu et al., 2013) and selected for predicted specificity score and on target activity. Genomic sequences recognized by the gRNAs are indicated below.

TABLE 2.1

| Guide ID | Genomic sequence (5′-3′) |
|---|---|
| AAVS1 gRNA | GTCACCAATCCTGTCCCTAGTGG |
| | (SEQ ID NO: 71) |
| IL2RG gRNA | ACTGGCCATTACAATCATGTGGG |
| | (SEQ ID NO: 72) |
| CD40LG gRNA | TGGATGATTGCACTTTATCAGGG |
| | (SEQ ID NO: 73) |

Ribonucleoproteins (RNPs) were assembled by incubating at 1:1.5 molar ratio SpCas9 protein (Aldevron) with synthetic cr:tracrRNA (Integrated DNA Technologies) for 10 minutes at 25° C. Electroporation enhancer (Integrated DNA Technologies) was added prior to electroporation according to manufacturer's instructions.

GSE56.WPRE, Ad5-E4orf6/7.WPRE and GSE56/Ad5-E4orf6/7.WPRE (which are disclosed in PCT/EP2019/066915) were synthetized with codon optimization for Homo sapiens (GeneArt™). Each construct was cloned in a pVax plasmid for mRNA in vitro transcription containing a T7 promoter, WPRE and 64bp-polyA. tTA.3′UTR was cloned in a pVax plasmid for mRNA in vitro transcription containing a T7 promoter, beta-globin 3′UTR and 64bp-polyA. The sequence of tTA mRNA is detailed in the Supplementary Material Section.

For mRNA in vitro transcription, pVax plasmid was linearized with the restriction enzyme (Spe I) and purified using phenol-chloroform. 5′-capped mRNA was in vitro transcribed using the commercial 5xMEGAscript T7 kit (Invitrogen). Synthetic RNA was purified using the RNeasy Plus Mini Kit (Qiagen) followed by HPLC column purification to remove contaminants. RNA was then concentrated using Amicon Ultra-15 (30K) (Millipore).

Gene Editing of K562 Cell Lines

The human K562 cells were maintained in Iscove's modified Dulbecco's medium (IMDM; Corning) supplemented with 10% fetal bovine serum (FBS; Euroclone), penicillin (100 IU/ml), streptomycin (100 μg/ml) and 2% glutamine. K562 cells were electroporated with 2.5-1.25 μM of RNPs. Transduction with AAV6 was performed at a dose of 1×10⁴ vg/cell 15 minutes after electroporation.

Gene Editing of Human CD34+ Cells

CD34+ cells were purchased frozen from Lonza. CD34+ cells were edited according to a previously optimized protocol (Schiroli et al., 2017). Briefly, 5×10⁵ CD34+ cells/ml were stimulated in serum-free StemSpan medium (StemCell Technologies) supplemented with penicillin, streptomycin, glutamine, 1 μM SR-1 (Biovision), 50 μM UM171 (STEMCell Technologies), 10 μM PGE2 added only at the beginning of the culture (Cayman), and human early-acting cytokines (SCF 100 ng/ml, Flt3-L 100 ng/ml, TPO 20 ng/ml, and IL-6 20 ng/ml; all purchased from Peprotech). After 3 days of pre-stimulation, cells were washed with PBS and electroporated using P3 Primary Cell 4D-Nucleofector X Kit and program EO-100 (Lonza). Cells were electroporated with 2.5-1.25 μM of RNPs. Transduction with AAV6 was performed at a dose of 1×10⁴ vg/cell 15′ after electroporation. GSE56 mRNA was utilized where indicated at a dose of 150 μg/ml, Ad5-E4orf6/7 mRNA was utilized where indicated at a dose of 75 or 150 μg/ml, GSE56/Ad5-E4orf6/7 mRNA was utilized where indicated at a dose of 215 μg/ml, tTA mRNA was utilized where indicated at a dose of 150 μg/ml. Gene editing efficiency was measured from cultured cells in vitro 3 days after electroporation by flow cytometry measuring the percentage of cells expressing the GFP/truncated NGFR/CXCR4 marker or by digital droplet PCR analysis designing primers and probe on the junction between the vector sequence and the targeted locus and on control sequences utilized as normalizer.

CD34+ HSPC Xenotransplantation Studies in NSG Mice

NOD-SCID-IL2Rg^{-/-} (NSG) mice were purchased from The Jackson Laboratory and were maintained in specific-pathogen-free (SPF) conditions. The procedures involving animals were designed and performed with the approval of the Animal Care and Use Committee of the San Raffaele Hospital (IACUC #749) and communicated to the Ministry of Health and local authorities according to Italian law.

For transplantation, 2×10⁵CD34+ cells treated for editing at day 4.5 of culture were injected intravenously into NSG mice after sub-lethal irradiation (150-180 cGy). Sample size was determined by the total number of available treated cells. Mice were attributed to each experimental group randomly. Human CD45+ cell engraftment and the presence of gene-edited cells were monitored by serial collection of blood from the mouse tail.

Molecular Analyses

For molecular analyses, genomic DNA was isolated with DNeasy Blood & Tissue Kit or QlAamp DNA Micro Kit (QIAGEN) according to the number of cells available. For digital droplet PCR analysis, 5-50 ng of genomic DNA were analyzed using the QX200 Droplet Digital PCR System (Biorad) according to the manufacturer's instructions. For HDR ddPCR, primers and probes were designed on the junction between the vector sequence and the targeted locus and on control sequences used for normalization (human TTC5 genes). Thermal conditions for annealing and extension were adjusted as follows: 55° C. for 30 sec, 72° C. for 2 min. Primers and probes for PCR and ddPCR amplifications are shown below.

TABLE 2.2

| Description | Orientation | Sequence (5′-3′) |
|---|---|---|
| Intron 1 IL2RG | FW | CTAGATTGGGGAGAAAATGA |
| 3′ integration | | (SEQ ID NO: 81) |
| junction ddPCR | | |
| | RV | GTGGGAAGGGGCCGTACAG |
| | | (SEQ ID NO: 82) |
| | Probe | GTAGCTCCTATGCTAGGCGT |
| | (FAM) | AGCC (SEQ ID NO: 83) |
| AAVS1 3′ | FW | GATTGGGAAGACAATAGCAG |
| integration | | (SEQ ID NO: 88) |
| junction ddPCR | | |
| | RV | TCTTGGGAAGTGTAAGGAAG |
| | | (SEQ ID NO: 86) |
| | Probe | CCAGATAAGGAATCTGCCTA |
| | (FAM) | (SEQ ID NO: 87) |
| CD40LG 3′ | FW | TTAGGAGGGGGTCTGATACA |
| integration | | (SEQ ID NO: 89) |
| junction ddPCR | | |
| | RV | TCCTCGATCTGTGGGAGGAA |
| | | GAGAA (SEQ ID NO: |
| | | 90) |
| | Probe | TCAGTCTCCCTCTGAGATGT |
| | (FAM) | (SEQ ID NO: 91) |
| Human TTC5 | (HEX) | PrimePCR ddPCR Copy |
| ddPCR | | Number Assay: TTC5, |
| | | Human (Biorad) |

Flow Cytometry

For immunophenotypic analyses (performed on FACSCanto II; BD Pharmingen), we used the antibodies listed below. Single stained and Fluorescence Minus One-stained cells were used as controls. 7-aminoactinomycin (Sigma Aldrich) was included in the sample preparation for flow cytometry according to the manufacturer's instructions to exclude dead cells from the analysis. Cell sorting was performed using MoFlo XDP Cell Sorter (Beckman Coulter) or FACSAria Fusion (BD Biosciences).

TABLE 2.3

| | Fluorochrome | Clone | Company |
|---|---|---|---|
| Anti-Human | | | |
| Antibody | | | |
| CD16/32 | none | | Miltenyi |
| | | | Biotec |
| CD133/2 | PE | 293C3 | Miltenyi |
| | | | Biotec |
| CD34 | PB | AC136 | Miltenyi |
| | | | Biotec |
| CD34 | PECy7 | 8G12 | BD |
| CD90 | APC | 5E10 | BD |
| CD132 | APC | TUGm2 | Biolegend |
| CD45 | PB | HI30 | Biolegend |
| CD45 | APCH7 | HI30 | eBioscience |
| CD184 | PECy7 | REA649 | Miltenyi |
| (CXCR4) | | | Biotec |
| CD184 | APC | REA649 | Miltenyi |
| (CXCR4) | | | Biotec |
| CD271 | PECy7 | ME20.4-1.H4 | Miltenyi |
| (NGFR) | | | Biotec |
| Anti-Mouse | | | |
| Antibody | | | |
| CD11b | APC | M1/70 | BD |
| CD16/32 | none | 2.4G2 | BD |
| CD19 | PeCy7 | 6D5 | Biolegend |
| CD3e | PE | 145-2C11 | BD |
| CD4 | PB | RM4-5 | BD |
| CD45.1 | FITC | A20 | BD |
| CD45.2 | PerCP-Cy5.5 | 104 | BD |
| CD8a | APC780 | 53-6.7 | eBioscience |
| Lineage | PE | | Biolegend |
| Cocktail | | | |
| Sca1 | PB | D7 | Biolegend |
| Sca1 | PeCy7 | D7 | BD |

Murine Lin-HSPC Competitive Transplantation

C57BL/6-Ly5.1 and Cd40lg^{-/-} (CD45.2) donor mice between 6 and 10 weeks of age were euthanized by CO₂ and bone marrow cells were retrieved from femurs, tibias, and humeri. HSPCs were purified by Lin- selection using the mouse Lineage Cell Depletion Kit (Miltenyi Biotec) according to the manufacturer's instructions. Cells were then cultured for 16 hours in serum-free StemSpan medium (StemCell Technologies) containing penicillin, streptomycin, glutamine, 200 ng/ml B18R Recombinant Protein (eBiovision) and a combination of mouse cytokines (20 ng/ml IL-3, 100 ng/ml SCF, 100 ng/ml Flt-3L, 50 ng/ml TPO all from Peprotech), at a concentration of 10⁶ cells/ml. Lin- were mixed at the indicated ratios and transplanted at the indicated doses into 8-week-old lethally irradiated Cd40lg^{-/-} mice.

Results

To assess whether transient delivery of tTA can activate selector expression, we performed targeted integration of the SMArT-D donor construct in the intron 1 of the IL2RG gene in K562 cell lines. At 14 days after targeting we observed undetectable levels of basal expression in mock-electroporated cells. On the contrary, cells electroporated 9 days after editing with different doses (0.25μg, 1 μg or 3 μg) of tTA mRNA showed efficient transactivation (FIG. 2.1B).

We then tested our platform in cord blood (CB)-derived CD34+ cells by co-delivering with the editing machinery tTA transactivator expressed as mRNA (FIG. 2.2A). FACS analyses 24 and 48 hours after editing showed low but detectable levels of GFP expression in edited CD34+ cells. Adeno Associated Viral vector (AAV)-transduced HSPCs electroporated with tTA mRNA in the presence or not of IL2RG Cas9 ribonucleoprotein (RNP) (here the guide RNA is for IL2RG) showed high and comparable GFP expression, indicating that tTA transactivate with similar efficiencies in both the integrated and not-integrated construct. Interestingly, doxycycline treatment upon electroporation efficiently controlled selector expression compared to doxycycline untreated cells. RNP-treated sample showed a major increase in the percentage and MFI of GFP+ cells compared to RNP-untreated ones, suggesting a preferential tTA activity on the integrated construct. However, only a small fraction of HDR-edited cells expressed the selector (FIG. 2.2B). Thus, we hypothesized that doxycycline withdrawal some hours after treatment could reawaken tTA activity and enable transient selector expression preferentially in targeted cells, while unintegrated AAV copies are going to be degraded and diluted. To this purpose, we tailored doxycycline doses and washout timing upon IL2RG editing and we compared GFP expression overtime in cells treated with AAV+tTA electroporated or not with RNP targeting the IL2RG locus (FIG. 2.2C). Doxycycline withdrawal at 12 hours post-editing did not allow efficient overexpression of the selector in targeted (RNP+AAV) cells neither in terms of percentage nor MFI of GFP+ cells, independently of the doxycycline doses. Importantly, AAV+tTA-treated cells incubated with the lower doxycycline dose, and then washed, still showed a lower percentage and MFI of selector+ cells compared to RNP+AAV+tTA washed control (FIG. 2.2D), confirming the preferential tTA activity on integrated constructs even at reduced doxycycline doses.

Interestingly, low dose doxycycline and delayed washout at 24 hours post-editing resulted in potent selector overexpression in the vast majority of HDR-edited cells, with no detectable transactivation in unintegrated cells upon withdrawal (FIG. 2.2E). Similar results were obtained with the lower doxycycline dose with 36 hours post-editing withdrawal (FIG. 2.2F). Overall, these results allowed to define the protocol in order to transient transactivate the selector gene integrated within the IL2RG locus in CD34+ cells: tTA is delivered together with RNP by electroporation after 3 days of pre-stimulation, doxycycline is added at 400 nM or 80 nM after AAV6 transduction washed out 24 h later (FIG. 2.3A). Metanalysis of multiple experiments showed that:
i) edited cells showed minimal but detectable basal levels of selector expression, due to the presence of the minimal promoter;
ii) doxycycline controlled more efficiently tTA activity on the non-integrated construct than in the integrated one;
iii) doxycycline washout allowed high transactivation (both in terms of percentage of positive cells and in terms of MFI) only within targeted cells.

Moreover, transactivation was present in almost all the edited cells (FIGS. 2.3B and 2.3C) and not only considering the bulk population but also the most primitive compartment (CD34+ CD133+ CD90+) (FIGS. 2.3D and 2.3E).

To demonstrate that our selection platform was portable to other relevant loci in CD34+ cells, we tested SMArT-D in the Adeno-Associated Virus Integration Site 1 (AAVS1) locus, a well-known safe harbor within the human genome, and we directly applied the protocol established in IL2RG (FIG. 2.4A). Similar to what we observed in the previous locus, we observed low level of basal expression of the selector gene from the integrated cassette. tTA, when present, was able to induce a high level of selector overexpression both in the non-integrated and in the integrated construct, while doxycycline (400 nM) particularly constrained transactivation in the cells that did not received RNP for targeted integration. Moreover, doxycycline washout at 24 h after the editing procedure resulted in an increased level of GFP expression limited to cells showing integration of the cassette (FIGS. 2.4B and 2.4C). Selector overexpression was confirmed also within the most primitive HSPC compartment (FIGS. 2.4D and 2.4E).

We next tested the SMArT-D strategy in another clinically relevant locus, CD40LG, the causative gene of HIGM syndrome. In this case, GFP selector was replaced with truncated NGFR to demonstrate the compatibility of our strategy with clinically compliant selectors. We took advantage of our established protocol (FIG. 2.5A) and we still observed the same trend of the previous loci: minimal level of basal expression from the integrated cassette without tTA, very limited transactivation in the presence of doxycycline (more pronounced in the integrated than in the non-integrated construct) and high selector overexpression only in edited cells after doxycycline withdrawal. These results were clear observing the percentage of NGFR positive cells (FIG. 2.5B), the MFI (FIG. 2.5C) and some representative FACS plots (FIG. 2.5D).

To improve the clinical applicability, we decided to test the same strategy replacing the marker gene (GFP or truncated NGFR) with a biological selector: in this way we can replace the in vitro selection (editing−>transient transactivation−>selection of edited cells through sorting) with an in vivo selection. In particular, we hypothesized that transient overexpression of the C-X-C chemokine receptor type 4 (CXCR4) only in HDR-edited HSPCs by SMArT-D strategy would enhance engraftment capacity of HDR-edited HSPCs conferring transient selective advantage in vivo against the NHEJ-edited or unedited counterpart. Indeed, CXCR4 is a biologically active protein involved in HSPCs migration and homing in bone marrow niche by sensitizing HSPCs to SDF1 (CXCL12) gradients (Ara et al., 2003). Moreover, CXCR4 overexpression has been shown to improve engraftment capacity of HSPCs (Kahn et al., 2004) (patent application PCT/EP2019/062666). We tested the SMArT-D strategy in the IL2RG locus both with CXCR4 wild type (WT) and mutant (WHIM) form, the latter characterized by a point mutation that increases CXCR4 stability on the membrane, possibly further prolonging CXCR4 overexpression on cell surface (Hernandez et al., 2003). For these experiments, we tested two different doses of doxycycline (400 nM and 80 nM) (FIG. 2.6A). We observed the doxycycline effect also with the lower dose tested and a transient transactivation after doxycycline wash only in the edited fraction with almost all the edited cells transactivated (FIGS. 2.6B and 2.6C). Moreover, looking at the CD34+ cells subpopulations we reached up to 13% of CXCR4-overexpressing cells in the most primitive compartment with the lower doxycycline dose (FIG. 2.6D). To confirm that CXCR4-overexpressing cells showed on target integration of the cassette, we measured targeted integration by HDR in sorted CXCR4^{high} cells and CXCR4^{low} and we found 100% of HDR efficiency in the CXCR4^{high} cells (FIG. 2.6E).

In addition, we tested whether SMArT-D strategy could be in principle combined with other strategies improving gene editing outcomes, such as those based on the use of the adenoviral protein (Ad5)-E4orf6/7, the p53 inhibitor GSE56 or their combination (PCT/EP2019/066915). Our results suggest that SMArT-D is compatible with all these platforms, without affecting transactivation efficiency (FIG. 2.7A).

To assess if SMArT-D manipulated HSPCs still preserve their engraftment capacity and if CXCR4-overexpressing HDR-edited cells were enriched in vivo, we transplanted 250,000 SMArT-D treated CB-derived CD34+ cells in NSG immunodeficient mice 36 h after the editing procedure, with a delay of 12 h compared with the standard procedure because of the necessity to washout doxycycline 24 h after gene editing. We had six different groups of mice:
(i) cells edited with CXCR4 WT construct and transiently transactivated with tTA;
(ii) cells edited with CXCR4 WHIM construct and transiently transactivated with tTA;
(iii) cells edited with CXCR4 WT construct and not transactivated.

Moreover, to possibly exploit a synergistic effect of p53 inhibition and CXCR4 overexpression we decided to test all the conditions in presence of GSE56:
(i) cells edited with CXCR4 WT construct, transiently transactivated with tTA and in presence of GSE56;
(ii) cells edited with CXCR4 WHIM construct, transiently transactivated with tTA and in presence of GSE56;
(iii) cells edited with CXCR4 WT construct, not transactivated but in presence of GSE56.

Mice were monitored by performing FACS analyses and molecular assays for HDR efficiency on blood samples collected at 6, 9, 12 and 18 weeks after transplantation (FIG. 2.8A). Despite prolonged and complex ex vivo manipulation, SMArT-D transactivated HDR-edited cells were able to engraft at long-term in immunodeficient mice. However, we could not observe neither higher engraftment nor HDR efficiency in mice receiving tTA transactivated cells compared to not transactivated ones, with the main difference among groups mainly related to enhanced percentage of repopulating human cells in mice receiving GSE56-treated cells (FIGS. 2.8B and 2.8C).

Enrichment of a pure population of corrected cells would entail transplantation of a low number of stem cells compared to bulk treated-cell transplant. Therefore, we reasoned which disease settings could benefit from reconstitution of even a limited input of functional cells. We modelled “selection-like” procedure in a mouse model of HIGM1 disease, a primary immunodeficiency due to inactivating mutation of CD40LG gene and characterized by defective IgG production in response of non-self antigens. Lethally-irradiated Cd40lg knock-out (KO) mice were transplanted with a total amount of 1,000,000 of cells of which: i) 25% Cd40lg wild-type (WT) and 75% KO or ii) 50% WT and 50% KO. To mimic a “selection-like” setting, we transplanted the same amount of WT cells, either 250,000 or 500,000, but without competition of KO cells. As controls, we transplanted 1,000,000 of KO cells or 1,000,000 of WT cells. We perform TNP/KLH vaccination 12 weeks after transplantation and we collected serum samples at 14 weeks. Mice were boosted with the TNP/KLH antigen at 15 weeks and serum samples collected (FIGS. 2.9A and 2.9B). We did not observe significant differences in absolute count of cells in peripheral bloods between the conditions (FIG. 2.9C). We observed a rescue of the TNP-KLH humoral IgG response comparable to mice injected with 100% WT cells in the context of 50%-50% competitive transplantation, while 25%-75% group only showed a partial rescue. Importantly, total rescue was achieved in non-competitive settings, even by transplanting only 250,00 WT cells (FIG. 2.9D). These data establish the rationale for a selection strategy in the context of HSPC gene correction for HIGM syndrome.

### REFERENCES

Banaszynski, L. A., Chen, L. C., Maynard-Smith, L. A., Ooi, A. G., and Wandless, T. J. (2006). A rapid, reversible, and tunable method to regulate protein function in living cells using synthetic small molecules. Cell 126, 995-1004.
Chavez, A., Scheiman, J., Vora, S., Pruitt, B. W., Tuttle, M., P R Iyer, E., . . . Church, G. M. (2015). Highly efficient Cas9-mediated transcriptional programming. Nature Methods. https://doi.org/10.1038/nmeth.3312
Chavez, A., Tuttle, M., Pruitt, B. W., Ewen-Campen, B., Chari, R., Ter-Ovanesyan, D., . . . Church, G. (2016). Comparison of Cas9 activators in multiple species. Nature Methods. https://doi.org/10.1038/nmeth.3871
De Ravin, S. S., Li, L., Wu, X., Choi, U., Allen, C., Koontz, S., . . . Malech, H. L. (2017). CRISPR-Cas9 gene repair of hematopoietic stem cells from patients with X-linked chronic granulomatous disease. Science Translational Medicine. https://doi.org/10.1126/scitranslmed.aah3480
Dever, D. P., Bak, R. O., Reinisch, A., Camarena, J., Washington, G., Nicolas, C. E., . . . Porteus, M. H. (2016). CRISPR/Cas9 β-globin gene targeting in human haematopoietic stem cells. Nature. https://doi.org/10.1038/nature20134
Genovese, P., Schiroli, G., Escobar, G., Di Tomaso, T., Firrito, C., Calabria, A., . . . Naldini, L. (2014). Targeted genome editing in human repopulating haematopoietic stem cells. Nature. https://doi.org/10.1038/nature13420
Gilbert, L. A., Horlbeck, M. A., Adamson, B., Villalta, J. E., Chen, Y., Whitehead, E. H., . . . Weissman, J. S. (2014). Genome-Scale CRISPR-Mediated Control of Gene Repression and Activation. Cell. https://doi.org/10.1016/j.cell.2014.09.029
Ginn, S. L., Hallwirth, C. V., Liao, S. H. Y., Teber, E. T., Arthur, J. W., Wu, J., . . . Alexander, I. E. (2017). Limiting Thymic Precursor Supply Increases the Risk of Lymphoid Malignancy in Murine X-Linked Severe Combined Immunodeficiency. Molecular Therapy—Nucleic Acids. https://doi.org/10.1016/j.omtn.2016.11.011
Iwamoto, M., Björklund, T., Lundberg, C., Kirik, D., & Wandless, T. J. (2010). A general chemical method to regulate protein stability in the mammalian central nervous system. Chemistry and Biology, 17(9), 981-988. https://doi.org/10.1016/j.chembiol.2010.07.009
Konermann, S., Brigham, M. D., Trevino, A. E., Joung, J., Abudayyeh, O. O., Barcena, C., . . . Zhang, F. (2015). Genome-scale transcriptional activation by an engineered CRISPR-Cas9 complex. Nature. https://doi.org/10.1038/nature14136
Loew, R., Heinz, N., Hampf, M., Bujard, H., & Gossen, M. (2010). Improved Tet-responsive promoters with minimized background expression. BMC Biotechnology. https://doi.org/10.1186/1472-6750-10-81
Lombardo, A., Cesana, D., Genovese, P., Di Stefano, B., Provasi, E., Colombo, D. F., . . . Naldini, L. (2011). Site-specific integration and tailoring of cassette design for sustainable gene transfer. Nature Methods, 8(10), 861-869. https://doi.org/10.1038/nmeth.1674
Martins, V. C., Busch, K., Juraeva, D., Blum, C., Ludwig, C., Rasche, V., . . . Rodewald, H. R. (2014). Cell competition is a tumour suppressor mechanism in the thymus. Nature. https://doi.org/10.1038/nature13317
Phillips-Cremins, J. E., & Corces, V. G. (2013). Chromatin Insulators: Linking Genome Organization to Cellular Function. Molecular Cell. https://doi.org/10.1016/j.molcel.2013.04.018
Rakhit, R., Navarro, R., & Wandless, T. J. (2014). Chemical biology strategies for posttranslational control of protein function. Chemistry and Biology. https://doi.org/10.1016/j.chembiol.2014.08.011
Schiroli, G., Ferrari, S., Conway, A., Jacob, A., Capo, V., Albano, L., . . . Naldini, L. (2017). Preclinical modeling highlights the therapeutic potential of hematopoietic stem cell gene editing for correction of SCID-X1. Science Translational Medicine, 9(411). https://doi.org/10.1126/scitranslmed.aan0820
Wang, J., Exline, C. M., Declercq, J. J., Llewellyn, G. N., Hayward, S. B., Li, P. W. L., . . . Cannon, P. M. (2015). Homology-driven genome editing in hematopoietic stem and progenitor cells using ZFN mRNA and AAV6 donors. Nature Biotechnology. https://doi.org/10.1038/nbt.3408

Additional References
Ara, T., Tokoyoda, K., Sugiyama, T., Egawa, T., Kawabata, K., & Nagasawa, T. (2003). Long-term hematopoietic stem cells require stromal cell-derived factor-1 for colonizing bone marrow during ontogeny. Immunity.
De Ravin, S. S., Li, L., Wu, X., Choi, U., Allen, C., Koontz, S., . . . Malech, H. L. (2017). CRISPR-Cas9 gene repair of hematopoietic stem cells from patients with X-linked chronic granulomatous disease. Science Translational Medicine. https://doi.org/10.1126/scitranslmed.aah3480
Dever, D. P., Bak, R. O., Reinisch, A., Camarena, J., Washington, G., Nicolas, C. E., . . . Porteus, M. H. (2016). CRISPR/Cas9 β-globin gene targeting in human haematopoietic stem cells. Nature. https://doi.org/10.1038/nature20134
Genovese, P., Schiroli, G., Escobar, G., Di Tomaso, T., Firrito, C., Calabria, A., . . . Naldini, L. (2014). Targeted genome editing in human repopulating haematopoietic stem cells. Nature. https://doi.org/10.1038/nature13420
Gossen, M., & Bujard, H. (1992). Tight control of gene expression in mammalian cells by tetracycline-responsive promoters. Proceedings of the National Academy of Sciences of the United States of America. https://doi.org/10.1073/pnas.89.12.5547
Hernandez, P. A., Gorlin, R. J., Lukens, J. N., Taniuchi, S., Bohinjec, J., Francois, F., . . . Diaz, G. A. (2003). Mutations in the chemokine receptor gene CXCR4 are associated with WHIM syndrome, a combined immunodeficiency disease. Nature Genetics. https://doi.org/10.1038/ng 1149
Hsu, P. D., Scott, D. A., Weinstein, J. A., Ran, F. A., Konermann, S., Agarwala, V., . . . Zhang, F. (2013). DNA targeting specificity of RNA-guided Cas9 nucleases. Nature Biotechnology. https://doi.org/10.1038/nbt.2647
Kahn, J., Byk, T., Jansson-Sjostrand, L., Petit, I., Shivtiel, S., Nagler, A., . . . Lapidot, T. (2004). Overexpression of CXCR4 on human CD34+ progenitors increases their proliferation, migration, and NOD/SCID repopulation. Blood. https://doi.org/10.1182/blood-2003-07-2607
Schiroli, G., Conti, A., Ferrari, S., della Volpe, L., Jacob, A., Albano, L., . . . Di Micco, R. (2019). Precise Gene Editing Preserves Hematopoietic Stem Cell Function following Transient p53-Mediated DNA Damage Response. Cell Stem Cell. https://doi.org/10.1016/j.stem.2019.02.019
Schiroli, G., Ferrari, S., Conway, A., Jacob, A., Capo, V., Albano, L., . . . Naldini, L. (2017). Preclinical modeling highlights the therapeutic potential of hematopoietic stem cell gene editing for correction of SCID-X1. Science Translational Medicine. https://doi.org/10.1126/scitranslmed.aan0820
Wang, J., Exline, C. M., Declercq, J. J., Llewellyn, G. N., Hayward, S. B., Li, P. W. L., . . . Cannon, P. M. (2015). Homology-driven genome editing in hematopoietic stem and progenitor cells using ZFN mRNA and AAV6 donors. Nature Biotechnology. https://doi.org/10.1038/nbt.3408

References Cited in Materials and Methods (Example 1)
1. J. Wang, C. M. Exline, J. J. DeClercq, G. N. Llewellyn, S. B. Hayward, P. W. Li, D. A. Shivak, R. T. Surosky, P. D. Gregory, M. C. Holmes, P. M. Cannon, Homology-driven genome editing in hematopoietic stem and progenitor cells using ZFN mRNA and AAV6 donors. Nat Biotechnol 33, 1256-1263 (2015).
2. G. Schiroli, S. Ferrari, A. Conway, A. Jacob, V. Capo, L. Albano, T. Plati, M. C. Castiello, F. Sanvito, A. R. Gennery, C. Bovolenta, R. Palchaudhuri, D. T. Scadden, M. C. Holmes, A. Villa, G. Sitia, A. Lombardo, P. Genovese, L. Naldini, Preclinical modeling highlights the therapeutic potential of hematopoietic stem cell gene editing for correction of SCID-X1. Sci Transl Med 9, (2017).
3. C. D. Richardson, G. J. Ray, M. A. DeWitt, G. L. Curie, J. E. Corn, Enhancing homology-directed genome editing by catalytically active and inactive CRISPR-Cas9 using asymmetric donor DNA. Nat Biotechnol 34, 339-344 (2016).
4. P. D. Hsu, D. A. Scott, J. A. Weinstein, F. A. Ran, S. Konermann, V. Agarwala, Y. Li, E. J. Fine, X. Wu, O. Shalem, T. J. Cradick, L. A. Marraffini, G. Bao, F. Zhang, DNA targeting specificity of RNA-guided Cas9 nucleases. Nat Biotechnol 31, 827-832 (2013).

All references cited in this specification are incorporated by reference into this specification.

SEQUENCES
sqRNA and TALE binding sites
See Tables 1 and 2.
(SEQ ID NOs: 1-40)
SMArT minimal promoters
T6-SK promoter
(SEQ ID NO: 41)
TCTAGAATTAGCTTTAGGCGTGTACGGTGGGCGCCTATAAAAGCAGAGCTCGTTTAGTG
AACCGTCAGATCGCCTGGAGCAATTCCACAACACTTTTGTCTTATACCAACTTTCCGTA
CCACTTCCTACCCTCGTAAAGAATCCGCGG
minCMV promoter
(SEQ ID NO: 42)
GTACGGTGGGAGGCCTATATAAGCAGAGCTCGTTTAGTGAACCGTCAGATCGCCTGGAG
ACGCCATCCACGCTGTTTTGACCTCCATAGAAGACACCGGGACCGATCCA
Selectors
eGFP
(SEQ ID NO: 43)
ATGGTGAGCAAGGGCGAGGAGCTGTTCACCGGGGTGGTGCCCATCCTGGTCGAGCTGGA
CGGCGACGTAAACGGCCACAAGTTCAGCGTGTCCGGCGAGGGCGAGGGCGATGCCACCT
ACGGCAAGCTGACCCTGAAGTTCATCTGCACCACCGGCAAGCTGCCCGTGCCCTGGCCC
ACCCTCGTGACCACCCTGACCTACGGCGTGCAGTGCTTCAGCCGCTACCCCGACCACAT
GAAGCAGCACGACTTCTTCAAGTCCGCCATGCCCGAAGGCTACGTCCAGGAGCGCACCA
TCTTCTTCAAGGACGACGGCAACTACAAGACCCGCGCCGAGGTGAAGTTCGAGGGCGAC
ACCCTGGTGAACCGCATCGAGCTGAAGGGCATCGACTTCAAGGAGGACGGCAACATCCT
GGGGCACAAGCTGGAGTACAACTACAACAGCCACAACGTCTATATCATGGCCGACAAGC
AGAAGAACGGCATCAAGGTGAACTTCAAGATCCGCCACAACATCGAGGACGGCAGCGTG
CAGCTCGCCGACCACTACCAGCAGAACACCCCCATCGGCGACGGCCCCGTGCTGCTGCC
CGACAACCACTACCTGAGCACCCAGTCCGCCCTGAGCAAAGACCCCAACGAGAAGCGCG
ATCACATGGTCCTGCTGGAGTTCGTGACCGCCGCCGGGATCACTCTCGGCATGGACGAG
CTGTACAAGTAA
NGFR
(SEQ ID NO: 44)
ATGGGGGCAGGTGCCACCGGCCGCGCCATGGACGGGCCGCGCCTGCTGCTGTTGCTGCT
TCTGGGGGTGTCCCTTGGAGGTGCCAAGGAGGCATGCCCCACAGGCCTGTACACACACA
GCGGTGAGTGCTGCAAAGCCTGCAACCTGGGCGAGGGTGTGGCCCAGCCTTGTGGAGCC
AACCAGACCGTGTGTGAGCCCTGCCTGGACAGCGTGACGTTCTCCGACGTGGTGAGCGC
GACCGAGCCGTGCAAGCCGTGCACCGAGTGCGTGGGGCTCCAGAGCATGTCGGCGCCGT
GCGTGGAGGCCGACGACGCCGTGTGCCGCTGCGCCTACGGCTACTACCAGGATGAGACG
ACTGGGCGCTGCGAGGCGTGCCGCGTGTGCGAGGCGGGCTCGGGCCTCGTGTTCTCCTG
CCAGGACAAGCAGAACACCGTGTGCGAGGAGTGCCCCGACGGCACGTATTCCGACGAGG
CCAACCACGTGGACCCGTGCCTGCCCTGCACCGTGTGCGAGGACACCGAGCGCCAGCTC
CGCGAGTGCACACGCTGGGCCGACGCCGAGTGCGAGGAGATCCCTGGCCGTTGGATTAC
ACGGTCCACACCCCCAGAGGGCTCGGACAGCACAGCCCCCAGCACCCAGGAGCCTGAGG
CACCTCCAGAACAAGACCTCATAGCCAGCACGGTGGCAGGTGTGGTGACCACAGTGATG
GGCAGCTCCCAGCCCGTGGTGACCCGAGGCACCACCGACAACCTCATCCCTGTCTATTG
CTCCATCCTGGCTGCTGTGGTTGTGGGCCTTGTGGCCTACATAGCCTTCAAGAGGTGGA
ACAGGGGGATCCTCTAG
ETTs
Sp-SdCas9
(SEQ ID NO: 45)
ATGGGCGGTAGGCGTGTACGGTGGGAGGTCTATATAAGCAGAGCTCGTTTAGTGAACCG
TCAGATCGCCTGGAGACGCCATCCACGCTGTTTTGACCTCCATAGAAGACACCGGGACC
GATCCAGCCTCCGGACTCTAGAGGATCGAACCCTTGCCACCATGGACAAGAAGTACTCC
ATTGGGCTCGCTATCGGCACAAACAGCGTCGGCTGGGCCGTCATTACGGACGAGTACAA
GGTGCCGAGCAAAAAATTCAAAGTTCTGGGCAATACCGATCGCCACAGCATAAAGAAGA
ACCTCATTGGCGCCCTCCTGTTCGACTCCGGGGAGACGGCCGAAGCCACGCGGCTCAAA
AGAACAGCACGGCGCAGATATACCCGCAGAAAGAATCGGATCTGCTACCTGCAGGAGAT
CTTTAGTAATGAGATGGCTAAGGTGGATGACTCTTTCTTCCATAGGCTGGAGGAGTCCT
TTTTGGTGGAGGAGGATAAAAAGCACGAGCGCCACCCAATCTTTGGCAATATCGTGGAC
GAGGTGGCGTACCATGAAAAGTACCCAACCATATATCATCTGAGGAAGAAGCTTGTAGA
CAGTACTGATAAGGCTGACTTGCGGTTGATCTATCTCGCGCTGGCGCATATGATCAAAT
TTCGGGGACACTTCCTCATCGAGGGGGACCTGAACCCAGACAACAGCGATGTCGACAAA
CTCTTTATCCAACTGGTTCAGACTTACAATCAGCTTTTCGAAGAGAACCCGATCAACGC
ATCCGGAGTTGACGCCAAAGCAATCCTGAGCGCTAGGCTGTCCAAATCCCGGCGGCTCG
AAAACCTCATCGCACAGCTCCCTGGGGAGAAGAAGAACGGCCTGTTTGGTAATCTTATC
GCCCTGTCACTCGGGCTGACCCCCAACTTTAAATCTAACTTCGACCTGGCCGAAGATGC
CAAGCTTCAACTGAGCAAAGACACCTACGATGATGATCTCGACAATCTGCTGGCCCAGA
TCGGCGACCAGTACGCAGACCTTTTTTTGGCGGCAAAGAACCTGTCAGACGCCATTCTG
CTGAGTGATATTCTGCGAGTGAACACGGAGATCACCAAAGCTCCGCTGAGCGCTAGTAT
GATCAAGCGCTATGATGAGCACCACCAAGACTTGACTTTGCTGAAGGCCCTTGTCAGAC
AGCAACTGCCTGAGAAGTACAAGGAAATTTTCTTCGATCAGTCTAAAAATGGCTACGCC
GGATACATTGACGGCGGAGCAAGCCAGGAGGAATTTTACAAATTTATTAAGCCCATCTT
GGAAAAAATGGACGGCACCGAGGAGCTGCTGGTAAAGCTTAACAGAGAAGATCTGTTGC
GCAAACAGCGCACTTTCGACAATGGAAGCATCCCCCACCAGATTCACCTGGGCGAACTG
CACGCTATCCTCAGGCGGCAAGAGGATTTCTACCCCTTTTTGAAAGATAACAGGGAAAA
GATTGAGAAAATCCTCACATTTCGGATACCCTACTATGTAGGCCCCCTCGCCCGGGGAA
ATTCCAGATTCGCGTGGATGACTCGCAAATCAGAAGAGACCATCACTCCCTGGAACTTC
GAGGAAGTCGTGGATAAGGGGGCCTCTGCCCAGTCCTTCATCGAAAGGATGACTAACTT
TGATAAAAATCTGCCTAACGAAAAGGTGCTTCCTAAACACTCTCTGCTGTACGAGTACT
TCACAGTTTATAACGAGCTCACCAAGGTCAAATACGTCACAGAAGGGATGAGAAAGCCA
GCATTCCTGTCTGGAGAGCAGAAGAAAGCTATCGTGGACCTCCTCTTCAAGACGAACCG
GAAAGTTACCGTGAAACAGCTCAAAGAAGACTATTTCAAAAAGATTGAATGTTTCGACT
CTGTTGAAATCAGCGGAGTGGAGGATCGCTTCAACGCATCCCTGGGAACGTATCACGAT
CTCCTGAAAATCATTAAAGACAAGGACTTCCTGGACAATGAGGAGAACGAGGACATTCT
TGAGGACATTGTCCTCACCCTTACGTTGTTTGAAGATAGGGAGATGATTGAAGAACGCT
TGAAAACTTACGCTCATCTCTTCGACGACAAAGTCATGAAACAGCTCAAGAGGCGCCGA
TATACAGGATGGGGGCGGCTGTCAAGAAAACTGATCAATGGGATCCGAGACAAGCAGAG
TGGAAAGACAATCCTGGATTTTCTTAAGTCCGATGGATTTGCCAACCGGAACTTCATGC
AGTTGATCCATGATGACTCTCTCACCTTTAAGGAGGACATCCAGAAAGCACAAGTTTCT
GGCCAGGGGGACAGTCTTCACGAGCACATCGCTAATCTTGCAGGTAGCCCAGCTATCAA
AAAGGGAATACTGCAGACCGTTAAGGTCGTGGATGAACTCGTCAAAGTAATGGGAAGGC
ATAAGCCCGAGAATATCGTTATCGAGATGGCCCGAGAGAACCAAACTACCCAGAAGGGA
CAGAAGAACAGTAGGGAAAGGATGAAGAGGATTGAAGAGGGTATAAAAGAACTGGGGTC
CCAAATCCTTAAGGAACACCCAGTTGAAAACACCCAGCTTCAGAATGAGAAGCTCTACC
TGTACTACCTGCAGAACGGCAGGGACATGTACGTGGATCAGGAACTGGACATCAATCGG
CTCTCCGACTACGACGTGGCTGCTATCGTGCCCCAGTCTTTTCTCAAAGATGATTCTAT
TGATAATAAAGTGTTGACAAGATCCGATAAAGCTAGAGGGAAGAGTGATAACGTCCCCT
CAGAAGAAGTTGTCAAGAAAATGAAAAATTATTGGCGGCAGCTGCTGAACGCCAAACTG
ATCACACAACGGAAGTTCGATAATCTGACTAAGGCTGAACGAGGTGGCCTGTCTGAGTT
GGATAAAGCCGGCTTCATCAAAAGGCAGCTTGTTGAGACACGCCAGATCACCAAGCACG
TGGCCCAAATTCTCGATTCACGCATGAACACCAAGTACGATGAAAATGACAAACTGATT
CGAGAGGTGAAAGTTATTACTCTGAAGTCTAAGCTGGTCTCAGATTTCAGAAAGGACTT
TCAGTTTTATAAGGTGAGAGAGATCAACAATTACCACCATGCGCATGATGCCTACCTGA
ATGCAGTGGTAGGCACTGCACTTATCAAAAAATATCCCAAGCTTGAATCTGAATTTGTT
TACGGAGACTATAAAGTGTACGATGTTAGGAAAATGATCGCAAAGTCTGAGCAGGAAAT
AGGCAAGGCCACCGCTAAGTACTTCTTTTACAGCAATATTATGAATTTTTTCAAGACCG
AGATTACACTGGCCAATGGAGAGATTCGGAAGCGACCACTTATCGAAACAAACGGAGAA
ACAGGAGAAATCGTGTGGGACAAGGGTAGGGATTTCGCGACAGTCCGGAAGGTCCTGTC
CATGCCGCAGGTGAACATCGTTAAAAAGACCGAAGTACAGACCGGAGGCTTCTCCAAGG
AAAGTATCCTCCCGAAAAGGAACAGCGACAAGCTGATCGCACGCAAAAAAGATTGGGAC
CCCAAGAAATACGGCGGATTCGATTCTCCTACAGTCGCTTACAGTGTACTGGTTGTGGC
CAAAGTGGAGAAAGGGAAGTCTAAAAAACTCAAAAGCGTCAAGGAACTGCTGGGCATCA
CAATCATGGAGCGATCAAGCTTCGAAAAAAACCCCATCGACTTTCTCGAGGCGAAAGGA
TATAAAGAGGTCAAAAAAGACCTCATCATTAAGCTTCCCAAGTACTCTCTCTTTGAGCT
TGAAAACGGCCGGAAACGAATGCTCGCTAGTGCGGGCGAGCTGCAGAAAGGTAACGAGC
TGGCACTGCCCTCTAAATACGTTAATTTCTTGTATCTGGCCAGCCACTATGAAAAGCTC
AAAGGGTCTCCCGAAGATAATGAGCAGAAGCAGCTGTTCGTGGAACAACACAAACACTA
CCTTGATGAGATCATCGAGCAAATAAGCGAATTCTCCAAAAGAGTGATCCTCGCCGACG
CTAACCTCGATAAGGTGCTTTCTGCTTACAATAAGCACAGGGATAAGCCCATCAGGGAG
CAGGCAGAAAACATTATCCACTTGTTTACTCTGACCAACTTGGGCGCGCCTGCAGCCTT
CAAGTACTTCGACACCACCATAGACAGAAAGCGGTACACCTCTACAAAGGAGGTCCTGG
ACGCCACACTGATTCATCAGTCAATTACGGGGCTCTATGAAACAAGAATCGACCTCTCT
CAGCTCGGTGGAGACAGCAGGGCTGACCCCAAGTGA
VP160 domain
(SEQ ID NO: 46)
GATCCGGAGGCGGGGCGGACGCGCTGGACGATTTCGATCTCGACATGCTGGGTTCTGAT
GCCCTCGATGACTTTGACCTGGATATGTTGGGAAGCGACGCATTGGATGACTTTGATCT
GGACATGCTCGGCTCCGATGCTCTGGACGATTTCGATCTCGATATGTTAGGGTCAGACG
CACTGGATGATTTCGACCTTGATATGTTGGGAAGCGATGCCCTTGATGATTTCGACCTG
GACATGCTCGGCAGCGACGCCCTGGACGATTTCGATCTGGACATGCTGGGGTCCGATGC
CTTGGATGATTTTGACTTGGATATGCTGGGGAGTGATGCCCTGGACGACTTTGACCTGG
ACATGCTGGGCTCCGATGCGCTCGATGACTTCGATTTGGATATGTTGTATTGAAAGCTT
CTGA
VPR domain
(SEQ ID NO: 47)
AAGAAGAGGAAGGTGTCGCCAGGGATCCGTCGACTTGACGCGTTGATATCAACAAGTTT
GTACAAAAAAGCAGGCTACAAAGAGGCCAGCGGTTCCGGACGGGCTGACGCATTGGACG
ATTTTGATCTGGATATGCTGGGAAGTGACGCCCTCGATGATTTTGACCTTGACATGCTT
GGTTCGGATGCCCTTGATGACTTTGACCTCGACATGCTCGGCAGTGACGCCCTTGATGA
TTTCGACCTGGACATGCTGATTAACTCTAGAAGTTCCGGATCTCCGAAAAAGAAACGCA
AAGTTGGTAGCCAGTACCTGCCCGACACCGACGACCGGCACCGGATCGAGGAAAAGCGG
AAGCGGACCTACGAGACATTCAAGAGCATCATGAAGAAGTCCCCCTTCAGCGGCCCCAC
CGACCCTAGACCTCCACCTAGAAGAATCGCCGTGCCCAGCAGATCCAGCGCCAGCGTGC
CAAAACCTGCCCCCCAGCCTTACCCCTTCACCAGCAGCCTGAGCACCATCAACTACGAC
GAGTTCCCTACCATGGTGTTCCCCAGCGGCCAGATCTCTCAGGCCTCTGCTCTGGCTCC
AGCCCCTCCTCAGGTGCTGCCTCAGGCTCCTGCTCCTGCACCAGCTCCAGCCATGGTGT
CTGCACTGGCTCAGGCACCAGCACCCGTGCCTGTGCTGGCTCCTGGACCTCCACAGGCT
GTGGCTCCACCAGCCCCTAAACCTACACAGGCCGGCGAGGGCACACTGTCTGAAGCTCT
GCTGCAGCTGCAGTTCGACGACGAGGATCTGGGAGCCCTGCTGGGAAACAGCACCGATC
CTGCCGTGTTCACCGACCTGGCCAGCGTGGACAACAGCGAGTTCCAGCAGCTGCTGAAC
CAGGGCATCCCTGTGGCCCCTCACACCACCGAGCCCATGCTGATGGAATACCCCGAGGC
CATCACCCGGCTCGTGACAGGCGCTCAGAGGCCTCCTGATCCAGCTCCTGCCCCTCTGG
GAGCACCAGGCCTGCCTAATGGACTGCTGTCTGGCGACGAGGACTTCAGCTCTATCGCC
GATATGGATTTCTCAGCCTTGCTGGGCTCTGGCAGCGGCAGCCGGGATTCCAGGGAAGG
GATGTTTTTGCCGAAGCCTGAGGCCGGCTCCGCTATTAGTGACGTGTTTGAGGGCCGCG
AGGTGTGCCAGCCAAAACGAATCCGGCCATTTCATCCTCCAGGAAGTCCATGGGCCAAC
CGCCCACTCCCCGCCAGCCTCGCACCAACACCAACCGGTCCAGTACATGAGCCAGTCGG
GTCACTGACCCCGGCACCAGTCCCTCAGCCACTGGATCCAGCGCCCGCAGTGACTCCCG
AGGCCAGTCACCTGTTGGAGGATCCCGATGAAGAGACGAGCCAGGCTGTCAAAGCCCTT
CGGGAGATGGCCGATACTGTGATTCCCCAGAAGGAAGAGGCTGCAATCTGTGGCCAAAT
GGACCTTTCCCATCCGCCCCCAAGGGGCCATCTGGATGAGCTGACAACCACACTTGAGT
CCATGACCGAGGATCTGAACCTGGACTCACCCCTGACCCCGGAATTGAACGAGATTCTG
GATACCTTCCTGAACGACGAGTGCCTCTTGCATGCCATGCATATCAGCACAGGACTGTC
CATCTTCGACACATCTCTGTTTTGA
TALE7 DBD (SMArT)
(SEQ ID NO: 48)
AAACGGGCCCTCTAGACTCGAGCGGCCGCGCCACCATGGGAAAACCTATTCCTAATCCT
CTGCTGGGCCTGGATTCTACCGGAGGCATGGCCCCTAAGAAAAAGCGGAAGGTGGACGG
CGGAGTGGACCTGAGAACACTGGGATATTCTCAGCAGCAGCAGGAGAAGATCAAGCCCA
AGGTGAGATCTACAGTGGCCCAGCACCACGAAGCCCTGGTGGGACACGGATTTACACAC
GCCCACATTGTGGCCCTGTCTCAGCACCCTGCCGCCCTGGGAACAGTGGCCGTGAAATA
TCAGGATATGATTGCCGCCCTGCCTGAGGCCACACACGAAGCCATTGTGGGAGTGGGAA
AACAGTGGTCTGGAGCCAGAGCCCTGGAAGCCCTGCTGACAGTGGCCGGAGAACTGAGA
GGACCTCCTCTGCAGCTGGATACAGGACAGCTGCTGAAGATTGCCAAAAGGGGCGGAGT
GACCGCGGTGGAAGCCGTGCACGCCTGGAGAAATGCCCTGACGGGTGCCCCCCTGAACC
TGACCCCGGACCAAGTGGTGGCTATCGCCAGCCACGATGGCGGCAAGCAAGCGCTCGAA
ACGGTGCAGCGGCTGTTGCCGGTGCTGTGCCAGGACCATGGCCTGACTCCGGACCAAGT
GGTGGCTATCGCCAGCCACGATGGCGGCAAGCAAGCGCTCGAAACGGTGCAGCGGCTGT
TGCCGGTGCTGTGCCAGGACCATGGCCTGACCCCGGACCAAGTGGTGGCTATCGCCAGC
AACGGTGGCGGCAAGCAAGCGCTCGAAACGGTGCAGCGGCTGTTGCCGGTGCTGTGCCA
GGACCATGGCCTGACTCCGGACCAAGTGGTGGCTATCGCCAGCCACGATGGCGGCAAGC
AAGCGCTCGAAACGGTGCAGCGGCTGTTGCCGGTGCTGTGCCAGGACCATGGCCTGACC
CCGGACCAAGTGGTGGCTATCGCCAGCAACGGTGGCGGCAAGCAAGCGCTCGAAACGGT
GCAGCGGCTGTTGCCGGTGCTGTGCCAGGACCATGGCCTGACTCCGGACCAAGTGGTGG
CTATCGCCAGCCACGATGGCGGCAAGCAAGCGCTCGAAACGGTGCAGCGGCTGTTGCCG
GTGCTGTGCCAGGACCATGGCCTGACCCCGGACCAAGTGGTGGCTATCGCCAGCAACGG
TGGCGGCAAGCAAGCGCTCGAAACGGTGCAGCGGCTGTTGCCGGTGCTGTGCCAGGACC
ATGGCCTGACCCCGGACCAAGTGGTGGCTATCGCCAGCAATCACGGCGGCAAGCAAGCG
CTCGAAACGGTGCAGCGGCTGTTGCCGGTGCTGTGCCAGGACCATGGCCTGACCCCGGA
CCAAGTGGTGGCTATCGCCAGCAATCACGGCGGCAAGCAAGCGCTCGAAACGGTGCAGC
GGCTGTTGCCGGTGCTGTGCCAGGACCATGGCCTGACTCCGGACCAAGTGGTGGCTATC
GCCAGCCACGATGGCGGCAAGCAAGCGCTCGAAACGGTGCAGCGGCTGTTGCCGGTGCT
GTGCCAGGACCATGGCCTGACCCCGGACCAAGTGGTGGCTATCGCCAGCAACGGTGGCG
GCAAGCAAGCGCTCGAAACGGTGCAGCGGCTGTTGCCGGTGCTGTGCCAGGACCATGGC
CTGACTCCGGACCAAGTGGTGGCTATCGCCAGCCACGATGGCGGCAAGCAAGCGCTCGA
AACGGTGCAGCGGCTGTTGCCGGTGCTGTGCCAGGACCATGGCCTGACTCCGGACCAAG
TGGTGGCTATCGCCAGCCACGATGGCGGCAAGCAAGCGCTCGAAACGGTGCAGCGGCTG
TTGCCGGTGCTGTGCCAGGACCATGGCCTGACCCCGGACCAAGTGGTGGCTATCGCCAG
CAACATTGGCGGCAAGCAAGCGCTCGAAACGGTGCAGCGGCTGTTGCCGGTGCTGTGCC
AGGACCATGGCCTGACCCCGGACCAAGTGGTGGCTATCGCCAGCAACGGTGGCGGCAAG
CAAGCGCTCGAAACGGTGCAGCGGCTGTTGCCGGTGCTGTGCCAGGACCATGGCCTGAC
TCCGGACCAAGTGGTGGCTATCGCCAGCCACGATGGCGGCAAGCAAGCGCTCGAAACGG
TGCAGCGGCTGTTGCCGGTGCTGTGCCAGGACCATGGCCTGACCCCGGACCAAGTGGTG
GCTATCGCCAGCAATCACGGCGGCAAGCAAGCGCTCGAAACGGTGCAGCGGCTGTTGCC
GGTGCTGTGCCAGGACCATGGCCTGACCCCGGACCAAGTGGTGGCTATCGCCAGCAACG
GTGGCGGCAAGCAAGCGCTCGAAAGCATTGTGGCCCAGCTGAGCCGGCCTGATCCGGCG
TTGGCCGCGTTGACCAACGATCACCTGGTGGCCCTGGCCTGTCTGGGAGGCAGACCTGC
CCTGGATGCCGTGAAAAAAGGACTGCCTCACGCCCCTGCCCTGATCAAGAGAACAAATA
GAAGAATCCCCGAGCGGACCTCTCACAGAGTGGCCGGATCACA
TALE3 DBD (SMArTER)
(SEQ ID NO: 49)
GTGGACCTGAGAACACTGGGATATTCTCAGCAGCAGCAGGAGAAGATCAAGCCCAAGGT
GAGATCTACAGTGGCCCAGCACCACGAAGCCCTGGTGGGACACGGATTTACACACGCCC
ACATTGTGGCCCTGTCTCAGCACCCTGCCGCCCTGGGAACAGTGGCCGTGAAATATCAG
GATATGATTGCCGCCCTGCCTGAGGCCACACACGAAGCCATTGTGGGAGTGGGAAAACA
GTGGTCTGGAGCCAGAGCCCTGGAAGCCCTGCTGACAGTGGCCGGAGAACTGAGAGGAC
CTCCTCTGCAGCTGGATACAGGACAGCTGCTGAAGATTGCCAAAAGGGGCGGAGTGACC
GCGGTGGAAGCCGTGCACGCCTGGAGAAATGCCCTGACGGGTGCCCCCCTGAACCTGAC
CCCGGACCAAGTGGTGGCTATCGCCAGCAACAATGGCGGCAAGCAAGCGCTCGAAACGG
TGCAGCGGCTGTTGCCGGTGCTGTGCCAGGACCATGGCCTGACCCCGGACCAAGTGGTG
GCTATCGCCAGCAACGGTGGCGGCAAGCAAGCGCTCGAAACGGTGCAGCGGCTGTTGCC
GGTGCTGTGCCAGGACCATGGCCTGACTCCGGACCAAGTGGTGGCTATCGCCAGCCACG
ATGGCGGCAAGCAAGCGCTCGAAACGGTGCAGCGGCTGTTGCCGGTGCTGTGCCAGGAC
CATGGCCTGACCCCGGACCAAGTGGTGGCTATCGCCAGCAACATTGGCGGCAAGCAAGC
GCTCGAAACGGTGCAGCGGCTGTTGCCGGTGCTGTGCCAGGACCATGGCCTGACTCCGG
ACCAAGTGGTGGCTATCGCCAGCCACGATGGCGGCAAGCAAGCGCTCGAAACGGTGCAG
CGGCTGTTGCCGGTGCTGTGCCAGGACCATGGCCTGACCCCGGACCAAGTGGTGGCTAT
CGCCAGCAACATTGGCGGCAAGCAAGCGCTCGAAACGGTGCAGCGGCTGTTGCCGGTGC
TGTGCCAGGACCATGGCCTGACTCCGGACCAAGTGGTGGCTATCGCCAGCCACGATGGC
GGCAAGCAAGCGCTCGAAACGGTGCAGCGGCTGTTGCCGGTGCTGTGCCAGGACCATGG
CCTGACCCCGGACCAAGTGGTGGCTATCGCCAGCAACATTGGCGGCAAGCAAGCGCTCG
AAACGGTGCAGCGGCTGTTGCCGGTGCTGTGCCAGGACCATGGCCTGACCCCGGACCAA
GTGGTGGCTATCGCCAGCAATCACGGCGGCAAGCAAGCGCTCGAAACGGTGCAGCGGCT
GTTGCCGGTGCTGTGCCAGGACCATGGCCTGACTCCGGACCAAGTGGTGGCTATCGCCA
GCCACGATGGCGGCAAGCAAGCGCTCGAAACGGTGCAGCGGCTGTTGCCGGTGCTGTGC
CAGGACCATGGCCTGACCCCGGACCAAGTGGTGGCTATCGCCAGCAACATTGGCGGCAA
GCAAGCGCTCGAAACGGTGCAGCGGCTGTTGCCGGTGCTGTGCCAGGACCATGGCCTGA
CTCCGGACCAAGTGGTGGCTATCGCCAGCCACGATGGCGGCAAGCAAGCGCTCGAAACG
GTGCAGCGGCTGTTGCCGGTGCTGTGCCAGGACCATGGCCTGACCCCGGACCAAGTGGT
GGCTATCGCCAGCAACATTGGCGGCAAGCAAGCGCTCGAAACGGTGCAGCGGCTGTTGC
CGGTGCTGTGCCAGGACCATGGCCTGACCCCGGACCAAGTGGTGGCTATCGCCAGCAAC
GGTGGCGGCAAGCAAGCGCTCGAAACGGTGCAGCGGCTGTTGCCGGTGCTGTGCCAGGA
CCATGGCCTGACCCCGGACCAAGTGGTGGCTATCGCCAGCAACATTGGCGGCAAGCAAG
CGCTCGAAACGGTGCAGCGGCTGTTGCCGGTGCTGTGCCAGGACCATGGCCTGACCCCG
GACCAAGTGGTGGCTATCGCCAGCAACGGTGGCGGCAAGCAAGCGCTCGAAACGGTGCA
GCGGCTGTTGCCGGTGCTGTGCCAGGACCATGGCCTGACCCCGGACCAAGTGGTGGCTA
TCGCCAGCAACGGTGGCGGCAAGCAAGCGCTCGAAACGGTGCAGCGGCTGTTGCCGGTG
CTGTGCCAGGACCATGGCCTGACCCCGGACCAAGTGGTGGCTATCGCCAGCAACGGTGG
CGGCAAGCAAGCGCTCGAAAGCATTGTGGCCCAGCTGAGCCGGCCTGATCCGGCGTTGG
CCGCGTTGACCAACGATCACCTGGTGGCCCTGGCCTGTCTGGGAGGCAGACCTGCCCTG
GATGCCGTGAAAAAAGGACTGCCTCACGCCCCTGCCCTGATCAAGAGAACAAATAGAAG
AATCCCCGAGCGGACCTCTCACAGAGTGGCC
Other elements of AAVS1-targeting constructs
Left homology arm
(SEQ ID NO: 50)
CCACTGTGGGGTGGAGGGGACAGATAAAAGTACCCAGAACCAGAGCCACATTAACCGGC
CCTGGGAATATAAGGTGGTCCCAGCTCGGGGACACAGGATCCCTGGAGGCAGCAAACAT
GCTGTCCTGAAGTGGACATAGGGGCCCGGGTTGGAGGAAGAAGACTAGCTGAGCTCTCG
GACCCCTGGAAGATGCCATGACAGGGGGCTGGAAGAGCTAGCACAGACTAGAGAGGTAA
GGGGGGTAGGGGAGCTGCCCAAATGAAAGGAGTGAGAGGTGACCCGAATCCACAGGAGA
ACGGGGTGTCCAGGCAAAGAAAGCAAGAGGATGGAGAGGTGGCTAAAGCCAGGGAGACG
GGGTACTTTGGGGTTGTCCAGAAAAACGGTGATGATGCAGGCCTACAAGAAGGGGAGGC
GGGACGCAAGGGAGACATCCGTCGGAGAAGGCCATCCTAAGAAACGAGAGATGGCACAG
GCCCCAGAAGGAGAAGGAAAAGGGAACCCAGCGAGTGAAGACGGCATGGGGTTGGGTGA
GGGAGGAGAGATGCCCGGAGAGGACCCAGACACGGGGAGGATCCGCTCAGAGGACATCA
CGTGGTGCAGCGCCGAGAAGGAAGTGCTCCGGAAAGAGCATCCTTGGGCAGCAACACAG
CAGAGAGCAAGGGGAAGAGGGAGTGGAGGAAGACGGAACCTGAAGGAGGCGGCAGGGAA
GGATCTGGGCCAGCCGTAGAGGTGACCCAGGCCACAAGCTGCAGACAGAAAGCGGCACA
GGCCCAGGGGAGAGAATGCTGGTCAGAGAAAGCA
SV40polyA
(SEQ ID NO: 51)
GAGATCCAGACATGATAAGATACATTGATGAGTTTGGACAAACCAAAACTAGAATGCAG
TGAAAAAAATGCCTTATTTGTGAAATTTGTGATGCTATTGCCTTATTTGTAACCATTAT
AAGCTGCAATAAACAAGTTAACAACAACAATTGCATTCATTTTATGTTTCAGGTTCAGG
GGGAGGTGTGGGAGGTTTTTTAAAGCAAGTAAAACCTCTACAAATGTGGTATGGCTGAT
TATGATCAGATCTCTCGAGG
Shortened left homology arm
(SEQ ID NO: 52)
GCAAGGAGAGAGATGGCTCCAGGAAATGGGGGTGTGTCACCAGATAAGGAATCTGCCTA
ACAGGAGGTGGGGGTTAGACCCAATATCAGGAGACTAGGAAGGAGGAGGCCTAAGGATG
GGGCTTTTCTGTCACCAATCCTGTCCCTA
Other elements of IL2RG-targeting constructs
Left homology arm
(SEQ ID NO: 53)
AGAGGAAACGTGTGGGTGGGGAGGGGTAGTGGGTGAGGGACCCAGGTTCCTGACACAGA
CAGACTACACCCAGGGAATGAAGAGCAAGCGCCATGTTGAAGCCATCATTACCATTCAC
ATCCCTCTTATTCCTGCAGCTGCCCCTGCTGGGAGTGGGGCTGAACACGACAATTCTGA
CGCCCAATGGGAATGAAGACACCACAGCTGGTGGGAAATCTGGGACTGGAGGGGGCTGG
TGAGAAGGGTGGCTGTGGGAAGGGGCCGTACAGAGATCTGGTGCCTGCCACTGG
IL2RG recoded corrective cDNA
(for intron 1 gene correction strategy)
(SEQ ID NO: 54)
ATTTCTTTCTGACCACCATGCCCACCGACAGCCTGAGCGTGAGCACCCTGCCCCTGCCC
GAGGTGCAGTGCTTCGTGTTCAACGTGGAGTACATGAACTGCACCTGGAACAGCAGCAG
CGAGCCCCAGCCCACCAATCTGACCCTGCACTACTGGTACAAGAACAGCGACAACGACA
AGGTGCAGAAGTGCAGCCACTACCTGTTCAGCGAGGAAATCACCAGCGGCTGCCAGCTG
CAGAAGAAAGAGATCCACCTGTACCAGACCTTCGTGGTGCAGCTGCAGGACCCCCGGGA
GCCCCGCAGGCAGGCCACCCAGATGCTGAAGCTGCAGAACCTGGTGATCCCCTGGGCCC
CTGAGAACCTGACACTGCACAAGCTGTCCGAGAGCCAGCTGGAACTGAACTGGAACAAC
CGCTTCCTGAACCACTGCCTGGAACACCTGGTGCAGTACCGGACCGACTGGGACCACAG
CTGGACCGAGCAGAGCGTGGACTACCGGCACAAGTTCAGCCTGCCCAGCGTGGACGGCC
AGAAGCGGTACACCTTCAGAGTGCGGAGCCGGTTCAACCCCCTGTGCGGCAGCGCCCAG
CACTGGTCCGAGTGGAGCCACCCCATCCACTGGGGCAGCAACACCAGCAAAGAGAACCC
CTTCCTGTTCGCCCTGGAAGCCGTGGTGATCAGCGTGGGCAGCATGGGCCTGATCATCT
CCCTGCTGTGCGTGTACTTCTGGCTGGAACGGACCATGCCCAGAATCCCCACCCTGAAG
AACCTGGAAGATCTGGTGACCGAGTACCACGGCAACTTCAGCGCCTGGTCCGGCGTGAG
CAAGGGCCTGGCCGAGAGCCTGCAGCCCGACTACAGCGAGCGGCTGTGCCTGGTGTCCG
AGATCCCCCCCAAAGGCGGAGCCCTGGGCGAAGGCCCTGGCGCCAGCCCCTGCAACCAG
CACAGCCCCTACTGGGCCCCTCCTTGCTACACCCTGAAGCCCGAGACCCGGGCCAAGCG
ATCCGGATCCGGAGCCACCAACTTCAGCCTGCTGAAGCAGGCCGGCGACGTGGAGGAGA
ACCCCGGCCCCTGA
Furin site + self-cleaving 2A peptide
(SEQ ID NO: 55)
CGGGCCAAGCGATCCGGATCCGGAGCCACCAACTTCAGCCTGCTGAAGCAGGCCGGCGA
CGTGGAGGAGAACCCCGGCCCC
Right homology arm
(SEQ ID NO: 56)
TACAATCATGTGGGCAGAATTGAAAAGTGGAGTGGGAAGGGCAAGGGGGAGGGTTCCCT
GCCTCACGCTACTTCTTCTTTCTTTCTTGTTTGTTTGTTTCTTTCTTTCTTTTGAGGCA
GGGTCTCACTATGTTGCCTAGGCTGGTCTCAAACTCCTGGCCTCTAGTGATCCTCCTGC
CTCAGCCTTTCAAAGCACCAGGATTACAGACATGAGCCACCGTGCTTGGCCTCCTCCTT
CTGACCATCATTTCTCTTTCCCTCCCTGCCT
PGK promoter
(SEQ ID NO: 57)
CCACGGGGTTGGGGTTGCGCCTTTTCCAAGGCAGCCCTGGGTTTGCGCAGGGACGCGGC
TGCTCTGGGCGTGGTTCCGGGAAACGCAGCGGCGCCGACCCTGGGTCTCGCACATTCTT
CACGTCCGTTCGCAGCGTCACCCGGATCTTCGCCGCTACCCTTGTGGGCCCCCCGGCGA
CGCTTCCTGCTCCGCCCCTAAGTCGGGAAGGTTCCTTGCGGTTCGCGGCGTGCCGGACG
TGACAAACGGAAGCCGCACGTCTCACTAGTACCCTCGCAGACGGACAGCGCCAGGGAGC
AATGGCAGCGCGCCGACCGCGATGGGCTGTGGCCAATAGCGGCTGCTCAGCGGGGCGCG
CCGAGAGCAGCGGCCGGGAAGGGGCGGTGCGGGAGGCGGGGTGTGGGGCGGTAGTGTGG
GCCCTGTTCCTGCCCGCGCGGTGTTCCGCATTCTGCAAGCCTCCGGAGCGCACGTCGGC
AGTCGGCTCCCTCGTTGACCGAATCACCGACCTCTCTCCCCAGG
SUPPLEMENTARY MATERIAL SECTION
tTA and 3′ UTR:
(SEQ ID NO: 58)
ATGTCTAGACTGGACAAGAGCAAAGTCATAAACTCTGCTCTGGAATTACTCAATGAAGT
CGGTATCGAAGGCCTGACGACAAGGAAACTCGCTCAAAAGCTGGGAGTTGAGCAGCCTA
CCCTGTACTGGCACGTGAAGAACAAGCGGGCCCTGCTCGATGCCCTGGCAATCGAGATG
CTGGACAGGCATCATACCCACTTCTGCCCCCTGGAAGGCGAGTCATGGCAAGACTTTCT
GCGGAACAACGCCAAGTCATTCCGCTGTGCTCTCCTCTCACATCGCGACGGGGCTAAAG
TGCATCTCGGCACCCGCCCAACAGAGAAACAGTACGAAACCCTGGAAAATCAGCTCGCG
TTCCTGTGTCAGCAAGGCTTCTCCCTGGAGAACGCACTGTACGCTCTGTCCGCCGTGGG
CCACTTTACACTGGGCTGCGTATTGGAGGATCAGGAGCATCAAGTAGCAAAAGAGGAAA
GAGAGACACCTACCACCGATTCTATGCCCCCACTTCTGAGACAAGCAATTGAGCTGTTC
GACCATCAGGGAGCCGAACCTGCCTTCCTTTTCGGCCTGGAACTAATCATATGTGGCCT
GGAGAAACAGCTAAAGTGCGAAAGCGGCGGGCCGGCCGACGCCCTTGACGATTTTGACT
TAGACATGCTCCCAGCCGATGCCCTTGACGACTTTGACCTTGATATGCTGCCTGCTGAC
GCTCTTGACGATTTTGACCTTGACATGCTCCCCGGGTAAaagctcgctttcttgctgtc
caatttctattaaaggttcctttgttccctaagtccaactactaaactgggggatatta
tgaagggccttgagcatctggattctgcctaataaaaaacatttattttcattgctgcg
ctagaagctcgctttcttgctgtccaatttctattaaaggttcctttgttccctaagtc
caactactaaactgggggatattatgaagggccttgagcatctggattctgcctaataa
aaaacatttattttcattgctgcgggacattcttaatt
The UTR in SEQ ID NO: 58 is depicted in
lower case letters.
TetR has the nucleotide sequence:
(SEQ ID NO: 74)
Atgtctagactggacaagagcaaagtcataaactctgctctggaattactcaatgaagt
cggtatcgaaggcctgacgacaaggaaactcgctcaaaagctgggagttgagcagccta
ccctgtactggcacgtgaagaacaagcgggccctgctcgatgccctggcaatcgagatg
ctggacaggcatcatacccacttctgccccctggaaggcgagtcatggcaagactttct
gcggaacaacgccaagtcattccgctgtgctctcctctcacatcgcgacggggctaaag
tgcatctcggcacccgcccaacagagaaacagtacgaaaccctggaaaatcagctcgcg
ttcctgtgtcagcaaggcttctccctggagaacgcactgtacgctctgtccgccgtggg
ccactttacactgggctgcgtattggaggatcaggagcatcaagtagcaaaagaggaaa
gagagacacctaccaccgattctatgcccccacttctgagacaagcaattgagctgttc
gaccatcagggagccgaacctgccttccttttcggcctggaactaatcatatgtggcct
ggagaaacagctaaagtgcgaaagcggc
In SEQ ID NO: 58 the sequence of VP16
(gccgacgcccttgacgattttgacttagacatgctc -
SEQ ID NO: 75) is repeated 3 times.
AAV6 IL2RG HOMOLOGY ARMS
LEFT:
(SEQ ID NO: 53)
AGAGGAAACGTGTGGGTGGGGAGGGGTAGTGGGTGAGGGACCCAGGTTCCTGACACAGA
CAGACTACACCCAGGGAATGAAGAGCAAGCGCCATGTTGAAGCCATCATTACCATTCAC
ATCCCTCTTATTCCTGCAGCTGCCCCTGCTGGGAGTGGGGCTGAACACGACAATTCTGA
CGCCCAATGGGAATGAAGACACCACAGCTGGTGGGAAATCTGGGACTGGAGGGGGCTGG
TGAGAAGGGTGGCTGTGGGAAGGGGCCGTACAGAGATCTGGTGCCTGCCACTGG
RIGHT:
(SEQ ID NO: 56)
TACAATCATGTGGGCAGAATTGAAAAGTGGAGTGGGAAGGGCAAGGGGGAGGGTTCCCT
GCCTCACGCTACTTCTTCTTTCTTTCTTGTTTGTTTGTTTCTTTCTTTCTTTTGAGGCA
GGGTCTCACTATGTTGCCTAGGCTGGTCTCAAACTCCTGGCCTCTAGTGATCCTCCTGC
CTCAGCCTTTCAAAGCACCAGGATTACAGACATGAGCCACCGTGCTTGGCCTCCTCCTT
CTGACCATCATTTCTCTTTCCCTCCCTGCCT
AAV6 AAVS1 HOMOLOGY ARMS
LEFT:
(SEQ ID NO: 61)
TGCTTTCTCTGACCAGCATTCTCTCCCCTGGGCCTGTGCCGCTTTCTGTCTGCAGCTTG
TGGCCTGGGTCACCTCTACGGCTGGCCCAGATCCTTCCCTGCCGCCTCCTTCAGGTTCC
GTCTTCCTCCACTCCCTCTTCCCCTTGCTCTCTGCTGTGTTGCTGCCCAAGGATGCTCT
TTCCGGAGCACTTCCTTCTCGGCGCTGCACCACGTGATGTCCTCTGAGCGGATCCTCCC
CGTGTCTGGGTCCTCTCCGGGCATCTCTCCTCCCTCACCCAACCCCATGCCGTCTTCAC
TCGCTGGGTTCCCTTTTCCTTCTCCTTCTGGGGCCTGTGCCATCTCTCGTTTCTTAGGA
TGGCCTTCTCCGACGGATGTCTCCCTTGCGTCCCGCCTCCCCTTCTTGTAGGCCTGCAT
CATCACCGTTTTTCTGGACAACCCCAAAGTACCCCGTCTCCCTGGCTTTAGCCACCTCT
CCATCCTCTTGCTTTCTTTGCCTGGACACCCCGTTCTCCTGTGGATTCGGGTCACCTCT
CACTCCTTTCATTTGGGCAGCTCCCCTACCCCCCTTACCTCTCTAGTCTGTGCTAGCTC
TTCCAGCCCCCTGTCATGGCATCTTCCAGGGGTCCGAGAGCTCAGCTAGTCTTCTTCCT
CCAACCCGGGCCCCTATGTCCACTTCAGGACAGCATGTTTGCTGCCTCCAGGGATCCTG
TGTCCCCGAGCTGGGACCACCTTATATTCCCAGGGCCGGTTAATGTGGCTCTGGTTCTG
GGTACTTTTATCTGTCCCCTCCACCCCAC
RIGHT:
(SEQ ID NO: 62)
TAGGGACAGGATTGGTGACAGAAAAGCCCCATCCTTAGGCCTCCTCCTTCCTAGTCTCC
TGATATTGGGTCTAACCCCCACCTCCTGTTAGGCAGATTCCTTATCTGGTGACACACCC
CCATTTCCTGGAGCCATCTCTCTCCTTGCCAGAACCTCTAAGGTTTGCTTACGATGGAG
CCAGAGAGGATCCTGGGAGGGAGAGCTTGGCAGGGGGTGGGAGGGAAGGGGGGGATGCG
TGACCTGCCCGGTTCTCAGTGG
AAV6 CD40LG HOMOLOGY ARMS
LEFT:
(SEQ ID NO: 63)
TGCTTTAAAAGTAAGCTATTTTTTTATGGAGACAGCTTTTTTCTTTTAAATTTCCAGCT
AGGCAAGAAGAGCGTCAATTTGATCTAAAATTTCATAATGCTTCAGATTAACATAGACA
TGGATAAGTCCCAGAATTTGCAGTCTTTTAGTAAAAGTAGCATTTTCTGTGTAATTCTT
CACAAGCACTGATTGTAGTTGCAGGATGCTCAGTCTCCCTCTGAGATGTTTTACATTTT
TAAATGGTTAGACTTGCAGGAACAAAAGAGCAGAGTAACTTAGTAGGCTGTTTTGCATT
CTTAGGAAAAGAAAACCATCAGGACTTATTTTGTTTTCATGTATTTTTTCACTTCCACT
GAGGAGTATAATTGGCTGGTGTTGACAAAATACCAATCATAGATGTAAAGGAGAAAGTT
GATTAGTTTTCTGGCTGTTCCTAAAATTCTGGATGCAGGAACTGTGGCTAGAAAGCATC
TGGATGATTGCACTTTACCTTAGG
RIGHT:
(SEQ ID NO: 64)
CAGGGATACTTGAGTGTCCTCTCTTAGGATCTGGACCTAGAATTAATGTCATGAGATTT
TTCTAACAGGATAAGGTGAGGTAGTGAGGGCTGAAGTCATCCACTGGGTTATCCAAATA
TTAGGTTTCACTGCTGACAAAAGAGGGGGCTTCTGGTCTGGTTGGTTATTTGTGTTTGG
CCTGATGTGCTCTGTCAATCAAATGTATGGACATAGGCCTAGCTTCTAAAGGGGCAATA
GTGACCTCAGTGGACTGATATTTACCGTACTATTTACATGTGCTCTTAATTACAGCAGA
AGCTGCCAGCTAACTGAATCTTGTTTTGAATCTAAAAAATCTACTCTTAAAGCAAGAAA
ATGGTATAAAATTAGTTGATAATGCAAGTGAATTCTGTACATTTAATTATTCTAAGACA
TTGGAAAATAAAATATCTTGTTACTTTGAGGATAAAAGATGATTTCTTTAAAAATGCAA
ATGTTTTCTACAAATACTAAAGTTAAA
TETO7-SK:
SEQ ID NO: 65)
CCCTATCAGTGATAGAGAACGTATGAAGAGTTTACTCCCTATCAGTGATAGAGAACGTA
TGCAGACTTTACTCCCTATCAGTGATAGAGAACGTATAAGGAGTTTACTCCCTATCAGT
GATAGAGAACGTATGACCAGTTTACTCCCTATCAGTGATAGAGAACGTATCTACAGTTT
ACTCCCTATCAGTGATAGAGAACGTATATCCAGTTTACTCCCTATCAGTGATAGAGAAC
GTATAAGCTTTAGGCGTGTACGGTGGGCGCCTATAAAAGCAGAGCTCGTTTAGTGAACC
GTCAGATCGCCTGGAGCAATTCCACAACACTTTTGTCTTATACCAACTTTCCGTACCAC
TTCCTACCCTCGTAAAG
The TetO sequence in SEQ ID NO: 65 has
the sequence:
(SEQ ID NO: 76)
Ccctatcagtgatagaga
There are 7 TetO sequences in SEQ ID NO: 65
GFP:
(SEQ ID NO: 43)
ATGGTGAGCAAGGGCGAGGAGCTGTTCACCGGGGTGGTGCCCATCCTGGTCGAGCTGGA
CGGCGACGTAAACGGCCACAAGTTCAGCGTGTCCGGCGAGGGCGAGGGCGATGCCACCT
ACGGCAAGCTGACCCTGAAGTTCATCTGCACCACCGGCAAGCTGCCCGTGCCCTGGCCC
ACCCTCGTGACCACCCTGACCTACGGCGTGCAGTGCTTCAGCCGCTACCCCGACCACAT
GAAGCAGCACGACTTCTTCAAGTCCGCCATGCCCGAAGGCTACGTCCAGGAGCGCACCA
TCTTCTTCAAGGACGACGGCAACTACAAGACCCGCGCCGAGGTGAAGTTCGAGGGCGAC
ACCCTGGTGAACCGCATCGAGCTGAAGGGCATCGACTTCAAGGAGGACGGCAACATCCT
GGGGCACAAGCTGGAGTACAACTACAACAGCCACAACGTCTATATCATGGCCGACAAGC
AGAAGAACGGCATCAAGGTGAACTTCAAGATCCGCCACAACATCGAGGACGGCAGCGTG
CAGCTCGCCGACCACTACCAGCAGAACACCCCCATCGGCGACGGCCCCGTGCTGCTGCC
CGACAACCACTACCTGAGCACCCAGTCCGCCCTGAGCAAAGACCCCAACGAGAAGCGCG
ATCACATGGTCCTGCTGGAGTTCGTGACCGCCGCCGGGATCACTCTCGGCATGGACGAG
CTGTACAAGTAA
NGFR:
SEQ ID NO: 44 is DLNGFR. This sequence
(SEQ ID NO: 67) is DLNGFR codon optimized
for human expression.
(SEQ ID NO: 67)
ATGGGAGCTGGTGCTACCGGCAGAGCTATGGATGGACCTAGACTGCTGCTCCTGCTGCT
GCTCGGAGTTTCTCTTGGCGGAGCCAAAGAGGCCTGTCCTACCGGCCTGTATACACACT
CTGGCGAGTGCTGCAAGGCCTGCAATCTTGGAGAAGGCGTGGCACAGCCTTGCGGCGCT
AATCAGACAGTGTGCGAGCCTTGCCTGGACAGCGTGACCTTTAGCGACGTGGTGTCTGC
CACCGAGCCATGCAAGCCTTGTACCGAGTGTGTGGGCCTGCAGAGCATGTCTGCCCCTT
GTGTGGAAGCCGACGATGCCGTGTGTAGATGCGCCTACGGCTACTACCAGGACGAGACA
ACAGGCAGATGCGAGGCCTGTAGAGTGTGTGAAGCCGGCTCTGGACTGGTGTTCAGCTG
CCAAGACAAGCAGAACACCGTGTGCGAGGAATGCCCCGATGGCACCTATAGCGACGAGG
CCAACCATGTGGATCCCTGCCTGCCTTGTACTGTGTGCGAAGATACCGAGCGGCAGCTG
CGCGAGTGTACAAGATGGGCTGATGCCGAGTGCGAAGAGATCCCCGGCAGATGGATCAC
CAGAAGCACACCTCCAGAGGGCAGCGATAGCACAGCCCCTTCTACACAAGAGCCCGAGG
CTCCTCCTGAGCAGGATCTGATTGCCTCTACAGTGGCCGGCGTGGTCACAACAGTGATG
GGATCTTCTCAGCCCGTGGTCACCAGAGGCACCACCGACAATCTGATCCCCGTGTACTG
TAGCATCCTGGCCGCCGTGGTTGTGGGACTCGTGGCCTATATCGCCTTCAAGCGGTGGA
ACCGGGGCATCCTGTAA
CXCR4 WT:
(SEQ ID NO: 68)
GCCACCATGTCTATTCCTCTGCCCCTGCTGCAGATCTACACCAGCGACAACTACACCGA
GGAAATGGGCAGCGGCGACTACGACAGCATGAAGGAACCCTGCTTCCGGGAAGAGAACG
CCAACTTCAACAAGATCTTCCTGCCCACAATCTACAGCATCATCTTTCTGACCGGCATC
GTGGGCAACGGACTCGTGATCCTCGTGATGGGCTACCAGAAAAAGCTGCGGAGCATGAC
CGACAAGTACCGGCTGCACCTGAGCGTGGCCGACCTGCTGTTCGTGATCACCCTGCCTT
TCTGGGCCGTGGACGCCGTGGCCAATTGGTACTTCGGCAACTTCCTGTGCAAGGCCGTG
CACGTGATCTACACAGTGAACCTGTACAGCAGCGTGCTGATCCTGGCCTTCATCAGCCT
GGACAGATACCTGGCCATCGTGCACGCCACCAACAGCCAGCGGCCTAGAAAGCTGCTGG
CCGAGAAGGTGGTGTACGTGGGCGTGTGGATTCCCGCCCTGCTGCTGACCATCCCCGAC
TTCATCTTCGCCAACGTGTCCGAGGCCGACGACCGGTACATCTGCGACCGGTTCTACCC
CAACGACCTGTGGGTGGTGGTGTTCCAGTTCCAGCACATCATGGTGGGACTGATCCTGC
CTGGCATCGTGATTCTGAGCTGCTACTGCATCATCATCAGCAAGCTGAGCCACAGCAAG
GGCCACCAGAAGCGGAAGGCCCTGAAAACCACCGTGATCCTGATTCTGGCTTTCTTCGC
CTGCTGGCTGCCCTACTACATCGGCATCAGCATCGACAGCTTCATCCTGCTGGAAATCA
TCAAGCAGGGCTGCGAGTTCGAGAACACCGTGCACAAGTGGATCAGCATTACCGAGGCC
CTGGCCTTTTTCCACTGCTGCCTGAACCCTATCCTGTACGCCTTCCTGGGCGCCAAGTT
CAAGACCTCTGCCCAGCACGCCCTGACCAGCGTGTCCAGAGGAAGCAGCCTGAAGATCC
TGAGCAAGGGCAAGAGAGGCGGCCACAGCTCCGTGTCTACAGAGAGCGAGAGCAGCAGC
TTCCACAGCAGCTGA
The single nucleotide difference between
CXCR4 WT and CXCR4 WHIM is shown in bold
and underlined.
CXCR4 WHIM:
(SEQ ID NO: 69)
GCCACCATGTCTATTCCTCTGCCCCTGCTGCAGATCTACACCAGCGACAACTACACCGA
GGAAATGGGCAGCGGCGACTACGACAGCATGAAGGAACCCTGCTTCCGGGAAGAGAACG
CCAACTTCAACAAGATCTTCCTGCCCACAATCTACAGCATCATCTTTCTGACCGGCATC
GTGGGCAACGGACTCGTGATCCTCGTGATGGGCTACCAGAAAAAGCTGCGGAGCATGAC
CGACAAGTACCGGCTGCACCTGAGCGTGGCCGACCTGCTGTTCGTGATCACCCTGCCTT
TCTGGGCCGTGGACGCCGTGGCCAATTGGTACTTCGGCAACTTCCTGTGCAAGGCCGTG
CACGTGATCTACACAGTGAACCTGTACAGCAGCGTGCTGATCCTGGCCTTCATCAGCCT
GGACAGATACCTGGCCATCGTGCACGCCACCAACAGCCAGCGGCCTAGAAAGCTGCTGG
CCGAGAAGGTGGTGTACGTGGGCGTGTGGATTCCCGCCCTGCTGCTGACCATCCCCGAC
TTCATCTTCGCCAACGTGTCCGAGGCCGACGACCGGTACATCTGCGACCGGTTCTACCC
CAACGACCTGTGGGTGGTGGTGTTCCAGTTCCAGCACATCATGGTGGGACTGATCCTGC
CTGGCATCGTGATTCTGAGCTGCTACTGCATCATCATCAGCAAGCTGAGCCACAGCAAG
GGCCACCAGAAGCGGAAGGCCCTGAAAACCACCGTGATCCTGATTCTGGCTTTCTTCGC
CTGCTGGCTGCCCTACTACATCGGCATCAGCATCGACAGCTTCATCCTGCTGGAAATCA
TCAAGCAGGGCTGCGAGTTCGAGAACACCGTGCACAAGTGGATCAGCATTACCGAGGCC
CTGGCCTTTTTCCACTGCTGCCTGAACCCTATCCTGTACGCCTTCCTGGGCGCCAAGTT
CAAGACCTCTGCCCAGCACGCCCTGACCAGCGTGTCCAGAGGAAGCAGCCTGAAGATCC
TGAGCAAGGGCAAGTGAGGCGGCCACAGCTCCGTGTCTACAGAGAGCGAGAGCAGCAGC
TTCCACAGCAGCTGA
The single nucleotide difference between
CXCR4 WT and CXCR4 WHIM is shown in bold
and underlined.

### SUMMARY CLAUSES

The present invention is defined in the claims and the accompanying description. For convenience other aspects of the present invention are presented herein by way of numbered clauses.

1. A method for selecting genome edited cells and/or for the enrichment of genome edited cells in a population of cells comprising:
(a) introducing into a cell or a population of cells at least one first component, at least one second component and at least one third component; and
(b) selecting the genome edited cells which transiently express or transiently upregulate a nucleotide sequence encoding a selector;
wherein the first component is a donor reporter cassette comprising the nucleotide sequence encoding the selector and a nucleotide sequence of interest (NOI);
wherein the second component is an engineered transcriptional transactivator (ETT) polypeptide or a nucleotide sequence encoding an ETT polypeptide; wherein the ETT polypeptide comprises a DNA binding domain (DBD) and at least one transcription activator (TA) domain;
wherein the third component is a nuclease system comprising a genome targeted nuclease and, optionally, a guide RNA (gRNA) comprising at least one targeted genomic sequence;
wherein the ETT polypeptide is transiently present in the cell or population of cells or the nucleotide sequence encoding the ETT polypeptide is transiently expressed in the cell or population of cells; and
wherein the presence of the nuclease system in the cell or the population of cells enables the insertion of the nucleotide sequence encoding the selector and the NOI into a target locus and wherein the transient presence of the ETT polypeptide or the transient expression of the nucleotide sequence encoding the ETT polypeptide enables transient expression or transient upregulation of the inserted nucleotide sequence encoding the selector.

2. The method according to clause 1 wherein the donor reporter cassette sequentially comprises:
(i) a left homology arm (HA) comprising a nucleotide sequence homologous to a target locus;
(ii) the nucleotide sequence encoding the selector operably linked to a minimal promoter;
(iii) the NOI operably linked to a promoter; and
(iv) a right homology arm (HA) comprising a nucleotide sequence homologous to the target locus;

wherein the ETT polypeptide of the second component or the ETT polypeptide expressed by the second component activates the minimal promoter when the nucleotide sequence encoding the selector is inserted into the target locus.

3. The method according to clause 1 wherein the donor reporter cassette sequentially comprises:
(i) a left homology arm (HA) comprising a nucleotide sequence homologous to a target locus;
(ii) optionally, a splicing acceptor site (SA);
(iii) the NOI;
(iv) the nucleotide sequence encoding the selector operably linked to a minimal promoter; and
(v) a right homology arm (HA) comprising a nucleotide sequence homologous to the target locus;

wherein the ETT polypeptide of the second component or the ETT polypeptide expressed by the second component activates the minimal promoter when the nucleotide sequence encoding the selector is inserted into the target locus.

4. The method according to clause 1 wherein the donor reporter cassette sequentially comprises:
(i) a left homology arm (HA) comprising a nucleotide sequence homologous to a target locus;
(ii) optionally, a splicing acceptor site (SA);
(iii) the NOI;
(iv) optionally, a nucleotide sequence encoding a 2A self-cleaving peptide (2A) or an internal ribosome entry site (IRES) element;
(v) the nucleotide sequence encoding the selector, optionally the nucleotide sequence encoding the selector is operably linked to a minimal promoter; and
(vi) a right homology arm (HA) comprising a nucleotide sequence homologous to the target locus;

wherein the ETT polypeptide of the second component or the ETT polypeptide expressed by the second component activates an endogenous promoter in the target locus.

5. The method according to any one of the preceding clauses wherein the DBD is a Transcriptional Activator-Like Effector (TALE) DBD, a Zinc finger, catalytically inactive Cpf1 or catalytically inactive Cas (dCas) and wherein the TA domain is selected from the group consisting of VP16, VP64, VP128, VP160, VPR, p65, Rta, HSF1, SAM, and SunTag.

6. The method according to any one of the preceding clauses wherein the gRNA is capable of binding to one or more of the nucleotide sequences selected from the group consisting of SEQ ID NOs 1 to 31 and sequences having at least 75% identity thereto.

7. The method according to any one of the preceding clauses wherein the target locus is a safe harbour.

8. The method according to clause 7 wherein the target locus is adeno-associated virus integration site 1 (AAVS1), a common integration site (CIS) of lentiviral vectors, IL2RG, gp91phox, HBB, RAG1, CD40LG, TRAC, TRBC, STAT, PRF1, a gene encoding for a protein expressed in the skin (such as collagen, keratin, laminin, desmocolin, desmoplachine, desmoglein, placoglobin, placophylline, integrin or other proteins that are involved in desmosomes and hemidesmosomes) or another safe harbour genomic locus.

9. A kit comprising a first component, a second component and a third component as defined in any one of the preceding clauses and, optionally, a cell population.

10. A population of genome edited cells produced by the method according to any one of the preceding clauses.

11. A pharmaceutical composition comprising the population of genome edited cells according to clause 10.

12. A population of genome edited cells according to clause 10 for use in gene therapy.

13. A population of genome edited cells according to clause 10 for use in the treatment or prevention of X-linked Severe Combined Immunodeficiency (SCID-X1).

14. A population of genome edited cells according to clause 10 for use in hematopoietic stem cell transplantation (HSCT).

15. A population of genome edited cells according to clause 10 for use in tissue repair.

1. A method for selecting genome edited cells and/or for the enrichment of genome edited cells in a population of cells comprising:
(a) introducing into a cell or a population of cells at least one first component, at least one second component and at least one third component; and
(b) selecting the genome edited cells which transiently express or transiently upregulate a nucleotide sequence encoding a selector;
wherein the first component is a donor reporter cassette comprising the nucleotide sequence encoding the selector and a nucleotide sequence of interest (NOI) and, optionally, a minimal promoter operably linked to a regulatory element;
wherein the second component is an engineered transcriptional transactivator (ETT) polypeptide or a nucleotide sequence encoding an ETT polypeptide; wherein the ETT polypeptide comprises a DNA binding domain (DBD) and at least one transcription activator (TA) domain;
wherein the third component is a nuclease system comprising a genome targeted nuclease and, optionally, a guide RNA (gRNA) comprising at least one targeted genomic sequence;
wherein the ETT polypeptide is transiently present in the cell or population of cells or the nucleotide sequence encoding the ETT polypeptide is transiently expressed in the cell or population of cells; and
wherein the presence of the nuclease system in the cell or the population of cells enables the insertion of the nucleotide sequence encoding the selector and the NOI and, optionally, the minimal promoter operably linked to the regulatory element into a target locus and wherein the transient presence of the ETT polypeptide or the transient expression of the nucleotide sequence encoding the ETT polypeptide enables transient expression or transient upregulation of the inserted nucleotide sequence encoding the selector optionally when a modulator is present in the cell or population of cells or optionally when a modulator is not present in the cell or population of cells.

2. The method according to claim 1 wherein the donor reporter cassette sequentially comprises:
(i) a left homology arm (HA) comprising a nucleotide sequence homologous to a target locus;
(ii) the nucleotide sequence encoding the selector operably linked to a minimal promoter;
(iii) the NOI operably linked to a promoter; and
(iv) a right homology arm (HA) comprising a nucleotide sequence homologous to the target locus;
wherein the ETT polypeptide of the second component or the ETT polypeptide expressed by the second component activates the minimal promoter when the nucleotide sequence encoding the selector is inserted into the target locus.

3. The method according to claim 1 wherein the donor reporter cassette sequentially comprises:
(i) a left homology arm (HA) comprising a nucleotide sequence homologous to a target locus;
(ii) optionally, a splicing acceptor site (SA);
(iii) the NOI;
(iv) the nucleotide sequence encoding the selector operably linked to a minimal promoter; and
(v) a right homology arm (HA) comprising a nucleotide sequence homologous to the target locus;
wherein the ETT polypeptide of the second component or the ETT polypeptide expressed by the second component activates the minimal promoter when the nucleotide sequence encoding the selector is inserted into the target locus.

4. The method according to claim 1 wherein the donor reporter cassette sequentially comprises:
(i) a left homology arm (HA) comprising a nucleotide sequence homologous to a target locus;
(ii) optionally, a splicing acceptor site (SA);
(iii) the NOI;
(iv) optionally, a nucleotide sequence encoding a 2A self-cleaving peptide (2A) or an internal ribosome entry site (IRES) element;
(v) the nucleotide sequence encoding the selector, optionally the nucleotide sequence encoding the selector is operably linked to a minimal promoter; and
(vi) a right homology arm (HA) comprising a nucleotide sequence homologous to the target locus;
wherein the ETT polypeptide of the second component or the ETT polypeptide expressed by the second component activates an endogenous promoter in the target locus.

5. The method according to claim 1 wherein the donor reporter cassette sequentially comprises:
(i) a left homology arm (HA) comprising a nucleotide sequence homologous to a target locus;
(ii) the NOI, optionally operably linked to a promoter;
(iii) the nucleotide sequence encoding the selector, wherein the nucleotide sequence encoding the selector is operably linked to a minimal promoter and the minimal promoter is operably linked to a regulatory element; and
(iv) a right homology arm (HA) comprising a nucleotide sequence homologous to the target locus;
wherein the ETT polypeptide of the second component or the ETT polypeptide expressed by the second component binds to the regulatory element and activates the minimal promoter when the nucleotide sequence encoding the selector is inserted into the target locus and when a modulator is present in the cell or population of cells; or
wherein the ETT polypeptide of the second component or the ETT polypeptide expressed by the second component binds to the regulatory element and activates the minimal promoter when the nucleotide sequence encoding the selector is inserted into the target locus and when a modulator is not present in the cell or population of cells.

6. The method according to claim 1 wherein the DBD is a Transcriptional Activator-Like Effector (TALE) DBD, a Zinc finger, catalytically inactive Cpf1 or catalytically inactive Cas (dCas) and wherein the TA domain is selected from the group consisting of VP16, VP64, VP128, VP160, VPR, p65, Rta, HSF1, SAM, and SunTag.

7. The method according to claim 5 wherein the DBD is a TetR or reverseTetR (rTetR) and wherein the TA domain is selected from the group consisting of VP16, VP64, VP128, VP160, VPR, p65, Rta, HSF1, SAM, and SunTag.

8. The method according to claim 1 wherein the gRNA is capable of binding to one or more of the nucleotide sequences selected from the group consisting of SEQ ID NOs 1 to 31 and sequences having at least 75% identity thereto.

9. The method according to claim 1 wherein the target locus is a safe harbour.

10. The method according to claim 9 wherein the target locus is adeno-associated virus integration site 1 (AAVS1), a common integration site (CIS) of lentiviral vectors, IL2RG, gp91phox, HBB, RAG1, CD40LG, TRAC, TRBC, STAT, PRF1, a gene encoding for a protein expressed in the skin (such as collagen, keratin, laminin, desmocolin, desmoplachine, desmoglein, placoglobin, placophylline, integrin or other proteins that are involved in desmosomes and hemidesmosomes) or another safe harbour genomic locus.

11. A kit comprising a first component, a second component and a third component as defined in claim 1 and, optionally, a cell population.

12. A population of genome edited cells produced by the method according to claim 1.

13. A pharmaceutical composition comprising the population of genome edited cells according to claim 12.

14.-17. (canceled)

18. A method of gene therapy comprising the step of administering a population of genome edited cells according to claim 12 to a subject in need thereof.

19. A method of treating or preventing X-linked Severe Combined Immunodeficiency (SCID-X1) comprising the step of administering a population of genome edited cells according to claim 12 to a subject in need thereof.

20. A method of hematopoietic stem cell transplantation (HSCT) comprising the step of administering a population of genome edited cells according to claim 12 to a subject in need thereof.

21. A method of tissue repair comprising the step of administering a population of genome edited cells according to claim 12 to a subject in need thereof.
